(19) United States
(12) Patent Application Publication
(10) Pub. No.: US 2023/0365712 A1
(43) Pub. Date: Nov. 16, 2023

(54) NOTCH4 ANTIBODIES AND USES THEREOF

(71) Applicant: The Children's Medical Center Corporation, Boston, MA (US)

(72) Inventors: Talal Chatila, Belmont, MA (US); Hani Harb, Boston, MA (US); Mehdi Benamar, Boston, MA (US)

(73) Assignee: The Children's Medical Center Corporation, Boston, MA (US)

(21) Appl. No.: 18/030,733

(22) PCT Filed: Oct. 15, 2021
(86) PCT No.: PCT/US2021/055181

§ 371 Date: Apr. 6, 2023

Related U.S. Application Data
(60) Provisional application No. 63/092,872, filed on Oct. 16, 2020.
(60) Provisional application No. 63/171,904, filed on Apr. 7, 2021.
(60) Provisional application No. 63/181,018, filed on Apr. 28, 2021.

(51) Int. Cl. C07K 16/44; C12N 15/63; A61P 29/00

(52) U.S. Cl. CPC C07K 16/44; C12N 15/63; A61P 29/00; C07K 2317/565

(57) ABSTRACT

Provided herein are anti-Notch4 antibodies or antigen binding fragments thereof and their use in the treatment of a disease or disorder associated with increased levels of Notch4.

### CROSS-REFERENCE TO RELATED APPLICATIONS

This application is a 35 U.S.C. § 371 National Phase Entry Application of International Application No. PCT/US2021/055181 filed Oct. 15, 2021, which designated the U.S., which claims benefit under 35 U.S.C. § 119(e) of U.S. Provisional Application No. 63/092,872 filed Oct. 16, 2020; U.S. Provisional Application No. 63/171,904 filed Apr. 7, 2021, and U.S. Provisional Application No. 63/181,018 filed Apr. 28, 2021, the contents of each of which are incorporated herein by reference in their entireties.

### GOVERNMENT SUPPORT

This invention was made with government support under Grant Number A1065617, awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Nov. 30, 2021, is named 701039-190050WOPT_SL.txt and is 46,799 bytes in size.

### TECHNICAL FIELD

The disclosure relates generally to anti-Notch4 antibodies or antigen binding fragments thereof and their use in the treatment of diseases or disorders associated with increased levels of Notch4.

### BACKGROUND

Coronavirus disease 2019 (Covid19), caused by the severe acute respiratory syndrome coronavirus 2 (SARS-CoV-2), is associated with lung inflammation and respiratory failure. In a prospective multi-country cohort of Covid19 patients, we found that increased Notch4 expression on circulating Treg cells was associated with increased disease severity, predicted mortality, and declined upon recovery. Deletion of Notch4 in Treg cells or therapy with anti-Notch4 antibodies in conventional and humanized mice suppressed the dysregulated innate immune response and rescued disease morbidity and mortality induced by a synthetic analogue of viral RNA or by the influenza H1N1 virus in an amphiregulin-dependent manner. Notably, amphiregulin production declined in Covid19 subjects as a function of disease severity and Notch4 expression. These results identify Notch4 as an immune regulatory switch that licenses virus-induced lung inflammation by altering Treg cell-mediated tissue repair. They also indicated Notch4 as a therapeutic target in Covid19 and other respiratory viral infections.

### SUMMARY

The methods and compositions provided herein are based, in part, on the optimization of anti-Notch4 antibodies for binding and inhibiting Notch4 in a cell, e.g., a regulatory T cell (Treg).

Accordingly, one aspect provided herein provides an antibody or antigen binding fragment that binds to Notch4 and comprises:
a. a heavy chain variable region (V_{H}) comprising an amino acid sequence selected from the group consisting of SEQ ID NOs 2, 4, or 6; or
b. a heavy chain variable region (V_{H}) comprising a nucleotide sequence selected from the group consisting of SEQ ID NOs 3, 5, or 7; or
c. a light chain variable region (V_{L}) comprising an amino acid sequence selected from the group consisting of SEQ ID NOs 8, 10, 12 or 14; or
d. a light chain variable region (V_{L}) comprising a nucleotide sequence selected from the group consisting of SEQ ID NOs 9, 11, 13, or 15.

In one embodiment of any aspect herein, the antibody or antigen-binding fragment comprises a nanobody, an scFv, a monoclonal antibody, a humanized antibody, a human antibody, a recombinant antibody, a chimeric antibody, or a Fab fragment.

In one embodiment of any aspect herein, the heavy chain variable region comprises a complementarity determining region 1 (CDR_H1) comprising an amino acid sequence that differs by no more than four amino acids from an amino acid sequence selected from the group consisting of: SEQ ID NOs.: 16, 22, 28, and 34.

In one embodiment of any aspect herein, the heavy chain variable region comprises a complementarity determining region 1 (CDR_H2) comprising an amino acid sequence that differs by no more than four amino acids from SEQ ID NO: 17, 23, 29, and 35.

In one embodiment of any aspect herein, the heavy chain variable region comprises a complementarity determining region 1 (CDR_H3) comprising an amino acid sequence that differs by no more than four amino acids from an amino acid sequence selected from the group consisting of: SEQ ID NOs.: 18, 24, 30, and 36.

In one embodiment of any aspect herein, the light chain variable region comprises a complementarity determining region 1 (CDR_L1) comprising an amino acid sequence that differs by no more than four amino acids from an amino acid sequence selected from the group consisting of: SEQ ID NOs.: SEQ ID NOs: 19, 25, 31, and 37.

In one embodiment of any aspect herein, the light chain variable region comprises a complementarity determining region 1 (CDR_L2) comprising an amino acid sequence that differs by no more than two amino acids from an amino acid sequence selected from the group consisting of: SEQ ID NOs.: 20, 26, 32, and 38.

In one embodiment of any aspect herein, the light chain variable region comprises a complementarity determining region 1 (CDR_L3) comprising an amino acid sequence that differs by no more than four amino acids from an amino acid sequence selected from the group consisting of: SEQ ID NOs.: 21, 27, 33, and 39.

In one embodiment of any aspect herein, the heavy chain variable region comprises a CDR_H1 comprising an amino acid sequence that differs by no more than four amino acids from SEQ ID NO: 16, a CDR_H2 comprising an amino acid sequence that differs by no more than four amino acids from SEQ ID NO: 17, and a CDR_H3 comprising an amino acid sequence that differs by no more than four amino acids from SEQ ID NO: 18.

In one embodiment of any aspect herein, the heavy chain variable region comprises a CDR_H1 comprising an amino acid sequence that differs by no more than four amino acids from SEQ ID NO: 22, a CDR_H2 comprising an amino acid sequence that differs by no more than four amino acids from SEQ ID NO: 23, and a CDR_H3 comprising an amino acid sequence that differs by no more than four amino acids from SEQ ID NO: 24.

In one embodiment of any aspect herein, the heavy chain variable region comprises a CDR_H1 comprising an amino acid sequence that differs by no more than four amino acids from SEQ ID NO: 28, a CDR_H2 comprising an amino acid sequence that differs by no more than four amino acids from SEQ ID NO: 29, and a CDR_H3 comprising an amino acid sequence that differs by no more than four amino acids from SEQ ID NO: 30.

In one embodiment of any aspect herein, the heavy chain variable region comprises a CDR_H1 comprising an amino acid sequence that differs by no more than four amino acids from SEQ ID NO: 34, a CDR_H2 comprising an amino acid sequence that differs by no more than four amino acids from SEQ ID NO: 35, and a CDR_H3 comprising an amino acid sequence that differs by no more than four amino acids from SEQ ID NO: 36.

In one embodiment of any aspect herein, the light chain variable region comprises a CDR_L1 comprising an amino acid sequence that differs by no more than four amino acids from SEQ ID NO: 19, a CDR_L2 comprising an amino acid sequence that differs by no more than four amino acids from SEQ ID NO: 20, and a CDR_L3 comprising an amino acid sequence that differs by no more than four amino acids from SEQ ID NO: 21.

In one embodiment of any aspect herein, the light chain variable region comprises a CDR_L1 comprising an amino acid sequence that differs by no more than four amino acids from SEQ ID NO: 25, a CDR_L2 comprising an amino acid sequence that differs by no more than four amino acids from SEQ ID NO: 26, and a CDR_L3 comprising an amino acid sequence that differs by no more than four amino acids from SEQ ID NO: 27.

In one embodiment of any aspect herein, the light chain variable region comprises a CDR_L1 comprising an amino acid sequence that differs by no more than four amino acids from SEQ ID NO: 31, a CDR_L2 comprising an amino acid sequence that differs by no more than four amino acids from SEQ ID NO: 32, and a CDR_L3 comprising an amino acid sequence that differs by no more than four amino acids from SEQ ID NO: 33.

In one embodiment of any aspect herein, the light chain variable region comprises a CDR_L1 comprising an amino acid sequence that differs by no more than four amino acids from SEQ ID NO: 37, a CDR_L2 comprising an amino acid sequence that differs by no more than four amino acids from SEQ ID NO: 38, and a CDR_L3 comprising an amino acid sequence that differs by no more than four amino acids from SEQ ID NO: 39.

In one embodiment of any aspect herein, the heavy chain variable region comprises an amino acid sequence having at least 85%, identity to an amino acid sequence selected from the group consisting of SEQ ID NOs: 2, 4, or 6.

In one embodiment of any aspect herein, the heavy chain variable region comprises a nucleotide sequence having at least 85%, identity to a nucleotide sequence selected from the group consisting of SEQ ID NOs: 3, 5, or 7.

In one embodiment of any aspect herein, the light chain variable region comprises an amino acid sequence having at least 85%, identity to an amino acid sequence selected from the group consisting of SEQ ID NOs: 8, 10, 12 or 14.

In one embodiment of any aspect herein, the light chain variable region comprises a nucleotide sequence having at least 85%, identity to a nucleotide sequence selected from the group consisting of SEQ ID NOs: 9, 11, 13, or 15.

In one embodiment of any aspect herein, the antibody or antigen binding fragment is a Notch4 inhibitor or a Notch4 neutralizing antibody.

In one embodiment of any aspect herein, the antibody or antigen binding fragment reduces Notch4 activity by at least 20% as compared to Notch4 activity in the absence of the antibody or antigen-binding fragment.

In one embodiment of any aspect herein, the antibody or antigen binding fragment disrupts binding of Notch4 to its cognate ligand.

Another aspect provided herein provides an antibody or antigen binding fragment that binds to Notch4 and comprises:
a. a heavy chain variable region (V_{H}) comprising:
   i. a complementarity determining region 1 (CDR_H1) sequence comprising an amino acid sequence that differs by no more than four amino acids from an amino acid sequence selected from the group consisting of SEQ ID NOs 16, 22, 28, and 34; or
   ii. a complementarity determining region 2 (CDR_H2) sequence comprising an amino acid sequence that differs by no more than four amino acids from an amino acid sequence selected from the group consisting of SEQ ID NOs 17, 23, 29, and 35; or
   iii. a complementarity determining region 3 (CDR_H3) sequence comprising an amino acid sequence that differs by no more than four amino acids from an amino acid sequence selected from the group consisting of SEQ ID NOs 18, 24, 30, and 36; or
b. a light chain variable region (V_{L}) comprising:
   i. a complementarity determining region 1 (CDR_L1) sequence comprising an amino acid sequence that differs by no more than four amino acids from an amino acid sequence selected from the group consisting of SEQ ID NOs 19, 25, 31, and 37; or
   ii. a complementarity determining region 2 (CDR_L2) sequence comprising an amino acid sequence that differs by no more than two amino acids from an amino acid sequence selected from the group consisting of SEQ ID NOs 20, 26, 32, and 38; or
   iii. a complementarity determining region 3 (CDR_L3) sequence comprising an amino acid sequence that differs by no more than four amino acids from an amino acid sequence selected from the group consisting of SEQ ID NOs 21, 27, 33, and 39.

In one embodiment of any aspect herein, the heavy chain variable region comprises an amino acid sequence having at least 91%, at least 92%, at least 93%, at least 94%, at least 95%, at least 96%, at least 97%, at least 98%, or at least 99% identity to an amino acid sequence selected from the group consisting of SEQ ID NOs: 2, 4, or 6.

In one embodiment of any aspect herein, the heavy chain variable region comprises an amino acid sequence selected from the group consisting of SEQ ID NOs: 2, 4, or 6.

In one embodiment of any aspect herein, the heavy chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 3, 5, or 7.

In one embodiment of any aspect herein, the heavy chain variable region comprises an amino acid sequence encoded by a nucleotide sequence selected from the group consisting of SEQ ID NOS: 3, 5, or 7.

In one embodiment of any aspect herein, the light chain variable region comprises an amino acid sequence having at least 91%, at least 92%, at least 93%, at least 94%, at least 95%, at least 96%, at least 97%, at least 98%, or at least 99% identity identity to an amino acid sequence selected from the group consisting of SEQ ID NOs: 8, 10, 12 or 14.

In one embodiment of any aspect herein, the light chain variable region comprises an amino acid sequence selected from the group consisting of SEQ ID NOs: 8, 10, 12, or 14.

In one embodiment of any aspect herein, the light chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 9, 11, 13, or 15.

In one embodiment of any aspect herein, the light chain variable region comprises an amino acid sequence encoded by a nucleotide sequence selected from the group consisting of SEQ ID NOs: 9, 11, 13, or 15.

In one embodiment of any aspect herein, the antibody comprising
a. a heavy chain variable region (V_{H}) comprising:
   i. a complementarity determining region 1 (CDR_H1) sequence comprising an amino acid sequence that differs by no more than four amino acids from an amino acid sequence selected from the group consisting of SEQ ID NOs 16, 22, 28, and 34;
   ii. a complementarity determining region 2 (CDR_H2) sequence comprising an amino acid sequence that differs by no more than four amino acids from an amino acid sequence selected from the group consisting of SEQ ID NOs 17, 23, 29, and 35;
   iii. a complementarity determining region 3 (CDR_H3) sequence comprising an amino acid sequence that differs by no more than four amino acids from an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence that differs by no more than four amino acids from an amino acid sequence selected from the group consisting of SEQ ID NOs 19, 25, 31, and 37;
   ii. a complementarity determining region 2 (CDR_L2) sequence comprising an amino acid sequence that differs by no more than two amino acids from an amino acid sequence selected from the group consisting of SEQ ID NOs 20, 26, 32, and 38;
   iii. a complementarity determining region 3 (CDR_L3) sequence comprising an amino acid sequence that differs by no more than four amino acids from an amino acid sequence selected from the group consisting of SEQ ID NOs 21, 27, 33, and 39.

In one embodiment of any aspect herein, the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:
a. a heavy chain variable region (V_{H}) comprising:
   i. a complementarity determining region 1 (CDR_H1) sequence comprising an amino acid sequence selected from the group consisting of SEQ ID NOs 16, 22, 28, and 34;
   ii. a complementarity determining region 2 (CDR_H2) sequence comprising an amino acid sequence selected from the group consisting of SEQ ID NOs 17, 23, 29, and 35;
   iii. a complementarity determining region 3 (CDR_H3) sequence comprising an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence selected from the group consisting of SEQ ID NOs 19, 25, 31, and 37;
   ii. a complementarity determining region 2 (CDR_L2) sequence comprising an amino acid sequence selected from the group consisting of SEQ ID NOs 20, 26, 32, and 38;
   iii. a complementarity determining region 3 (CDR_L3) sequence comprising an amino acid sequence selected from the group consisting of SEQ ID NOs 21, 27, 33, and 39.

In one embodiment of any aspect herein, the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:

the antibody or antigen binding fragment thereof comprise:
a. a heavy chain variable region (V_{H}) comprising:
   i. a complementarity determining region 1 (CDR_H1) sequence comprising an amino acid sequence selected from the group consisting of SEQ ID NOs 16, 22, 28, and 34;
   ii. a complementarity determining region 2 (CDR_H2) sequence comprising an amino acid sequence selected from the group consisting of SEQ ID NOs 17, 23, 29, and 35;
   iii. a complementarity determining region 3 (CDR_H3) sequence comprising an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence selected from the group consisting of SEQ ID NOs 19, 25, 31, and 37;
   ii. a complementarity determining region 2 (CDR_L2) sequence comprising an amino acid sequence selected from the group consisting of SEQ ID NOs 20, 26, 32, and 38;
   iii. a complementarity determining region 3 (CDR_L3) sequence comprising an amino acid sequence selected from the group consisting of SEQ ID NOs 21, 27, 33, and 39.

In one embodiment of any aspect herein, the antibody or antigen-binding fragment comprises a heavy chain variable region (V_{H}) and a light chain variable region (V_{L}), wherein the heavy chain variable region comprises complementarity determining regions CDR_H1, CDR_H2, and CDR_H3,

wherein the light chain variable region comprises CDR_L1, CDR_L2, and CDR_L3, and wherein
(a) (i) CDR_H1 comprises an amino acid sequence that differs by no more than two amino acid residues from that of SEQ ID NO: 16,
   (ii) CDR_H2 comprises an amino acid sequence that differs by no more than two amino acid residues from that of SEQ ID NO: 17,
   (iii) CDR_H3 comprises an amino acid sequence that differs by no more than two amino acid residues from that of SEQ ID NO: 18,
   (iv) CDR_L1 comprises an amino acid sequence that differs by no more than two amino acid residues from that of SEQ ID NO: 19,
   (v) CDR_L2 comprises an amino acid sequence that differs by no more than two amino acid residues from that of SEQ ID NO: 20; and
   (vi) CDR_L3 comprises an amino acid sequence that differs by no more than two amino acid residues from that of SEQ ID NO: 21; or
(b) (i) CDR_H1 comprises an amino acid sequence that differs by no more than two amino acid residues from that of SEQ ID NO: 22,
   (ii) CDR_H2 comprises an amino acid sequence that differs by no more than two amino acid residues from that of SEQ ID NO: 23,
   (iii) CDR_H3 comprises an amino acid sequence that differs by no more than two amino acid residues from that of SEQ ID NO: 24,
   (iv) CDR_L1 comprises an amino acid sequence that differs by no more than two amino acid residues from that of SEQ ID NO: 25,
   (v) CDR_L2 comprises an amino acid sequence that differs by no more than two amino acid residues from that of SEQ ID NO: 26; and
   (vi) CDR_L3 comprises an amino acid sequence that differs by no more than two amino acid residues from that of SEQ ID NO: 27; or
(c) (i) CDR_H1 comprises an amino acid sequence that differs by no more than two amino acid residues from that of SEQ ID NO: 28,
   (ii) CDR_H2 comprises an amino acid sequence that differs by no more than two amino acid residues from that of SEQ ID NO: 29,
   (iii) CDR_H3 comprises an amino acid sequence that differs by no more than two amino acid residues from that of SEQ ID NO: 30,
   (iv) CDR_L1 comprises an amino acid sequence that differs by no more than two amino acid residues from that of SEQ ID NO: 31,
   (v) CDR_L2 comprises an amino acid sequence that differs by no more than two amino acid residues from that of SEQ ID NO: 32; and
   (vi) CDR_L3 comprises an amino acid sequence that differs by no more than two amino acid residues from that of SEQ ID NO: 33; or
(d) (i) CDR_H1 comprises an amino acid sequence that differs by no more than two amino acid residues from that of SEQ ID NO: 34,
   (ii) CDR_H2 comprises an amino acid sequence that differs by no more than two amino acid residues from that of SEQ ID NO: 35,
   (iii) CDR_H3 comprises an amino acid sequence that differs by no more than two amino acid residues from that of SEQ ID NO: 36,
   (iv) CDR_L1 comprises an amino acid sequence that differs by no more than two amino acid residues from that of SEQ ID NO: 37,
   (v) CDR_L2 comprises an amino acid sequence that differs by no more than two amino acid residues from that of SEQ ID NO: 38; and
   (vi) CDR_L3 comprises an amino acid sequence that differs by no more than two amino acid residues from that of SEQ ID NO: 39.

In one embodiment of any aspect herein, the antibody or antigen-binding fragment comprises
(a) (i) CDR_H1 comprises an amino acid sequence of SEQ ID NO: 16,
   (ii) CDR_H2 comprises an amino acid sequence of SEQ ID NO: 17,
   (iii) CDR_H3 comprises an amino acid sequence of SEQ ID NO: 18,
   (iv) CDR_L1 comprises an amino acid sequence of SEQ ID NO: 19,
   (v) CDR_L2 comprises an amino acid sequence of SEQ ID NO: 20; and
   (vi) CDR_L3 comprises an amino acid sequence of SEQ ID NO: 21; or
(b) (i) CDR_H1 comprises an amino acid sequence of SEQ ID NO: 22,
   (ii) CDR_H2 comprises an amino acid sequence of SEQ ID NO: 23,
   (iii) CDR_H3 comprises an amino acid sequence of SEQ ID NO: 24,
   (iv) CDR_L1 comprises an amino acid sequence of SEQ ID NO: 25,
   (v) CDR_L2 comprises an amino acid sequence of SEQ ID NO: 26; and
   (vi) CDR_L3 comprises an amino acid sequence of SEQ ID NO: 27; or
(c) (i) CDR_H1 comprises an amino acid sequence of SEQ ID NO: 28,
   (ii) CDR_H2 comprises an amino acid sequence of SEQ ID NO: 29,
   (iii) CDR_H3 comprises an amino acid sequence of SEQ ID NO: 30,
   (iv) CDR_L1 comprises an amino acid sequence of SEQ ID NO: 31,
   (v) CDR_L2 comprises an amino acid sequence of SEQ ID NO: 32; and
   (vi) CDR_L3 comprises an amino acid sequence of SEQ ID NO: 33; or
(d) (i) CDR_H1 comprises an amino acid sequence of SEQ ID NO: 34,
   (ii) CDR_H2 comprises an amino acid sequence of SEQ ID NO: 35,
   (iii) CDR_H3 comprises an amino acid sequence of SEQ ID NO: 36,
   (iv) CDR_L1 comprises an amino acid sequence of SEQ ID NO: 37,
   (v) CDR_L2 comprises an amino acid sequence of SEQ ID NO: 38; and
   (vi) CDR_L3 comprises an amino acid sequence of SEQ ID NO: 39.

Another aspect provided herein provides a composition comprising any of the antibody or antigen binding fragment described herein.

In one embodiment of any aspect herein, the composition comprises a pharmaceutically acceptable excipient or carrier.

Another aspect provided herein provides a cell comprising any of the antibody or antigen binding fragments described herein.

Another aspect provided herein provides a polynucleotide comprising any of the nucleotide sequence encoding any antibody or antigen binding fragment described herein.

In one embodiment of any aspect herein, the polynucleotide is comprised in a vector.

Another aspect provided herein provides a composition comprising any of the polynucleotides described herein.

Another aspect provided herein provides a cell comprising any polynucleotide described herein.

Another aspect provided herein provides a kit comprising:
a. any of the antibodies or antigen binding fragments described herein;
b. any of the polynucleotides described herein;
c. any of the compositions described herein; or
d. any of the cells described herein.

In one embodiment of any aspect herein, the kit further comprises instructions for use thereof.

Another aspect provided herein provides a method for reducing Notch4 activity, the method comprising: administering any of the antibodies or antigen binding fragments described herein; any of the polynucleotides described herein; or any of the compositions described herein to a cell.

In one embodiment of any aspect herein, said administering to the cell is in vitro. In one embodiment of any aspect herein, said administering to the cell is in vivo. In one embodiment of any aspect herein, said administering to the cell is in a subject.

Another aspect provided herein provides a method for reducing at least one symptom of a disease or disorder associated with increased levels of Notch4, the method comprising administering a therapeutically effective amount of any of the antibodies or antigen binding fragments described herein; any of the polynucleotides described herein; or any of the compositions described herein to a subject in need thereof.

In some embodiments, the disease or disorder associated with increased level of Notch4 is associated with airway inflammation, e.g., chronic airway inflammation. In one embodiment of any aspect herein, the disease or disorder associated with an increased level of Notch4 is selected from the group consisting of asthma, allergic disease, and diseases or disorders associated with pulmonary viral infections (e.g., diseases or disorders associated with influenza virus infections, coronavirus infectious disease, etc.).

In one embodiment of any aspect herein, the method further comprises, prior to administering, a step of diagnosing the subject with the disease or disorder associated with an increased level of Notch4.

In one embodiment of any aspect herein, the at least one symptom comprises tissue inflammation.

In one embodiment of any aspect herein, the at least one symptom comprises a symptom selected from the group consisting of macrophage skewing, neutrophil infiltration, production of an inflammatory cytokine, and combinations thereof.

In one embodiment of any aspect herein, inflammatory cytokine is IL-6 or IFNγ.

In one embodiment of any aspect herein, the subject is a mammal. In one embodiment of any aspect herein, the subject is human.

### BRIEF DESCRIPTION OF THE DRAWINGS

FIGS. 1A-1G show increased expression of Notch4 on circulating Treg cells of COVID-19 subjects. FIGS. 1A-1D, Flow cytometric analysis, cell frequencies, absolute numbers and MFI of Notch4 expression in Treg cells (FIGS. 1A and 1B) and Teff cells (FIGS. 1C and 1D) of control subjects and patients with mild, moderate, severe or resolved COVID-19 (healthy controls n=37, mild patients n=20, moderate patients n=54, severe patients n=36, and convalescent subjects n=6). FIG. 1E, Serum concentrations of IL-6 in the different subject groups (healthy controls n=37, mild patients n=18, moderate patients n=45, and severe patients n=21). FIG. 1F, Correlation analysis of Notch4 expression on Treg cells of patient and control subjects as a function of serum IL-6 concentrations (n=121). FIG. 1G, serum concentrations of IFNa, IFN13, IFN1 IFNA, CXCL10, IL-113, IL-8, IL-10, IL-12 and TNF in control and patient subjects (healthy controls n=37, mild patients n=18, moderate patients n=45, and severe patients n=21). Each symbol represents one subject. Numbers in flow plots indicate percentages. Error bars indicate SEM. Statistical tests: *P<0.05, **P<0.01, ****P<0.0001 by one-way ANOVA with Dunnett's post hoc analysis (FIGS. 1A-1E, and 1G) and Pearson correlation analysis (FIG. 1D). Data representative of two or three independent experiments. +

FIGS. 2A-2M show protective effect of Notch4 inhibition in poly LC-induced lung injury. FIGS. 2A and 2B flow cytometric analysis (FIG. 2A) and cell frequencies, absolute numbers and MFI (FIG. 2B) of Notch4 expression in lung, mediastinal lymph node (medLN) and spleen Treg and Teff cells of Foxp3^{YFPCre} mice treated with either PBS or poly I:C once daily for 6 days. FIG. 2C, Notch4 expression on lung, medLN and spleen Treg cells of Foxp3^{YFPCre} mice. FIGS. 2D and 2E, Weight index (FIG. 2D) and peak weight loss (FIG. 2E) of Foxp3^{YFPCre} and Foxp3^{YFPCre}Notch4^{Δ/Δ} mice treated with either PBS or poly I:C together with isotype control or anti-Notch4 mAb. FIG. 2F, Hematoxylin and eosin-stained sections of lung tissues (200× magnification). FIG. 2G, Inflammation scores. FIG. 2H, AHR in response to methacholine. RI, responsiveness index (a measure of airway resistance). FIG. 2I, BAL fluid IL-6 concentrations FIG. 2J, Graphical representation of lung tissue neutrophils and M1 and M2 macrophages. FIG. 2K, M1 and M2 macrophage frequencies in cultures of Poly I:C-treated lung macrophages incubated with Treg cells from the indicated Poly I:C-treated mice. FIG. 2L, flow cytometric analysis of IL-6Ra expression in lung Notch4+ or Nottch4⁻ Treg cells of Foxp3^{YFPCre} mice treated with poly I:C. FIG. 2M, In vitro induction of Notch4 expression in Treg cell from the lungs or spleens of Poly I:C or PBS-treated Foxp3^{YFPCre} mice. Each symbol represents one mouse (n=5-15 per group). Numbers in flow plots indicate percentages. Error bars indicate SEM. Statistical tests: Student's T-test (b), one-way ANOVA with Dunnett's post hoc analysis (FIGS. 2E, 2G, 2I, 2J and 2K); Two-way ANOVA with Sidak's post hoc analysis (FIGS. 2C, 2D, 2H, and 2L). ***P<0.001, ****P<0.0001. Data pooled from two or three independent experiments.

FIGS. 3A-3J show protective effect of Notch4 inhibition in influenza A H1N1 virus infection. FIGS. 3A and 3B, Weight index (FIG. 3A) and peak weight loss (FIG. 3B) of Foxp3^{YFPCre} and Foxp3^{YFPCre}Notch4^{Δ/Δ} mice that were either sham-treated or infected with a 40 k pfu dose of H1N1 virus, either alone or together with an anti-Notch4 mAb, as indicated. Arrows indicate time of antibody treatment. FIG. 3C, BAL fluid IL-6 concentrations in the indicated groups. FIG. 3D, Hematoxylin and eosin-stained sections and inflammation score of lung tissues isolated from the indicated mouse groups (×200 magnification). FIG. 3E, Viral load [viral copies/g tissue] in the respective mouse groups (n=5 per group). FIG. 3F, Flow cytometric analysis and graphical representation of Notch4 expression in lung tissue Treg cells of the respective Foxp3^{YFPCre} and Foxp3^{YFPCre}Notch4^{Δ/Δ} mouse groups. FIGS. 3G-3H, Weight index (FIG. 3F) peak weight loss (FIG. 3G) and survival (FIG. 3H) of Foxp3^{YFPCre} mice that were either sham-treated or infected with a 40 k pfu dose of H1N1 virus, either alone or together with an anti-Notch4 mAb, as indicated. Arrows indicate time of antibody treatment. FIG. 3I, BAL fluid IL-6 and IFN1 concentration. FIG. 3J, Survival curve of Foxp3^{YFPCre} mice that were either sham-treated or infected with lethal dose of H1N1 virus, either alone or together with an anti-Notch4 or anti-IL-6Rα mAb, as indicated. Each symbol represents one mouse (n=5-23). Numbers in flow plots indicate percentages. Error bars indicate SEM. Statistical tests: One-way ANOVA with Dunnett's post hoc analysis (FIGS. 3B, 3C, 3F, 3I, and 3J); two-way ANOVA with Sidak's post hoc analysis (FIGS. 3A, 3D, 3E, and 3G). *P<0.05, ***P<0.001, ****P<0.0001. Data pooled from two or three independent experiments.

FIGS. 4A-4J show Notch4 licenses viral lung inflammation in humanized mice. FIG. 4A, Scheme of humanized mice infection with H1N1 influenza virus and treatment with neutralizing antihuman Notch4 mAbs. FIG. 4B, Weight index of mice that were infected with a sublethal dose of H1N1 virus and treated either with isotype control mAb or anti-Notch4 mAb 3B11 or 4H1, as indicated. FIGS. 4C and 4D, Hematoxylin and eosin-stained sections and inflammation score of lung tissues isolated from the indicated mouse groups (×200 magnification). FIG. 4E Flow cytometric analysis and frequencies of Notch4 expression in lung Treg cells of the respective groups. F, G, Flow cytometric analysis and graphical representation of lung tissue M1 (FIG. 4F) and M2 macrophages (FIG. 4G). FIG. 4H, Neutrophil infiltration in the lungs of the respective mouse groups. I, BAL fluid IL-6 and IFN1 concentrations. FIG. 4J, Viral load in the respective mouse groups, measured as viral copies per 100 ng lung tissue RNA. Each symbol represents one mouse (n=4-5 per group). Numbers in flow plots indicate percentages. Error bars indicate SEM. Statistical tests: Two-way ANOVA with Sidak's post hoc analysis (FIG. 4B); One-way ANOVA with Dunnett's post hoc analysis (FIGS. 4D-4J). *P<0.05, **P<0.01, ****P<0.0001.

FIGS. 5A-51I show Notch4 deficiency reprograms the lung Treg cell transcriptome of Poly I:C-treated mice. FIGS. 5A-5C, Volcano plot (FIG. 5A), heat map (FIG. 5B) and pathway analysis (FIG. 5C) of gene transcripts of lung Treg cells isolated from Foxp3^{YFPCre} and Foxp3^{YFPCre}Notch4^{Δ/Δ} mice treated with Poly I:C (n=4 and n=5, respectively). FIG. 5D, flow cytometric histograms and graphical representation of lung tissue Treg cell expression of CD25, Helios, CTLA4, ICOS, and OX40 in Foxp3^{YFPCre} and Foxp3^{YFPCre}Notch4^{Δ/Δ} mice sampled at day 7 post poly I:C treatment (n=5 for each time point). FIGS. 5E-5H, Flow cytometric analysis (FIGS. 5E and 5G) and graphical representation (FIGS. 5F and 5H) of Helios expression in Foxp3⁺Notch4⁺ and Foxp3⁺Notch4⁻ lung tissue Treg cells in Foxp3^{YFPCre} mice sampled at the indicated dates post poly I:C treatment (n=5 for each time point). Each symbol represents one mouse. Numbers in flow plots indicate percentages. Error bars indicate SEM. Statistical tests: Pairwise comparisons of differential gene expression were computed using DESeq2 (FIGS. 5A-5C); Student's unpaired two tailed t-test (FIG. 5D) and two-way ANOVA with Sidak's post hoc analysis (FIGS. 5F and 5H). *P<0.05, **P<0.01, ***P<0.001, ****P<0.0001.

FIGS. 6A-61 show Notch4 inhibition promotes an amphiregulin-dependent immunoregulatory program. FIG. 6A, flow cytometric analysis of amphiregulin expression in Treg cells from the lungs of Foxp3^{YFPCre} and Foxp3^{YFPCre}Notch4^{Δ/Δ} mice either sham-treated or treated with poly I:C, either alone or together with an anti-Notch4 mAb, as indicated. FIG. 6B, Graphical representation of amphiregulin expression in lung tissue Treg and Teff cells. FIG. 6C, BAL fluid amphiregulin concentrations. FIG. 6D, Amphiregulin expression in lung tissue Treg and Teff cells of Foxp3YFPCre and Foxp3^{YFPCre}Notch4Δ/Δ mice either sham-treated or infected with H1N1 influenza virus either alone or together with an anti-Notch4 mAb, as indicated. FIG. 6E, BAL fluid amphiregulin concentrations. FIG. 6F, Flow cytometric analysis and graphical representation of amphiregulin expression in Notch4+ or Notch4⁻ Treg cells from the lungs of Foxp3^{YFPCre} mice treated with either PBS or poly I:C at the indicated time points. FIGS. 6G and 6H, Flow cytometric analysis and graphical representation of IL18Rα, IL6Rα and ST2 expression in total (FIG. 6G) or amphiregulin⁺ (FIG. 6H) Treg cells from the lungs of PBS or Poly I:C-treated Foxp3^{YFPCre} either with an isotype control or an anti-Notch4 mAb, as indicated. FIG. 6I, In vitro induction of amphiregulin expression in Treg cell from the lungs or spleens of Poly I:C or PBS-treated Foxp3^{YFPCre} mice. Each symbol represents one mouse (n=5-8 per group). Numbers in flow plots indicate percentages. Error bars indicate SEM. Statistical tests: One-way ANOVA with Dunnett's post hoc analysis (FIGS. 6B, 6C, 6D, 6F, and 6G); two-way ANOVA with Sidak's post hoc analysis (FIGS. 6E and 6H). *P<0.05, **P<0.01, ***P<0.001, ****P<0.0001

FIGS. 7A-7K show lung Protection by Notch4 antagonism is amphiregulin-dependent. FIG. 7A, Weight index and peak weight loss of Foxp3^{YFPCre} mice either sham treated or treated with Poly I:C, either alone or together with recombinant amphiregulin. FIG. 7B, Hematoxylin and eosin-stained sections and inflammation score of lung tissues isolated from the indicated mouse groups (×200 magnification. FIG. 7C, Serum TRITC dextran in Poly I:C+ recombinant amphiregulin-treated Foxp3^{YFPCre} mice at 1 hour post intra-tracheal instillation. FIG. 7D, BAL fluid concentration of IL-6. FIG. 7E, Weight index and peak weight loss of Foxp3^{YFPCre} and Foxp3^{YFPCre}Notch4^{Δ/Δ} mice either sham-treated or treated with poly I:C, anti-Notch4 mAb and/or amphiregulin blocking peptide (bp), as indicated. FIG. 7F, Hematoxylin and eosin-stained lung sections (×200 magnification). FIG. 7G, Inflammation scores (n=5 per group). FIGS. 7H and 71, Lung neutrophils (FIG. 7H), M1 and M2 macrophages (FIG. 7I). FIG. 7J, Left panel: serum amphiregulin concentrations in healthy controls and COVID-19 patient groups (controls n=17, mild n=20, moderate n=49, severe n=32); Right panel: Pearson correlation of Treg cell Notch4 expression and serum amphiregulin concentration in the patient and control subjects (n=99). FIG. 7K, Pearson correlation of Notch4 and amphiregulin expression in Treg cell (right panel) and Teff cell (left panel) of COVID-19 patients (n=39). Each symbol represents one mouse (n=5-10 per group). Numbers in flow plots indicate percentages. Error bars indicate SEM. Statistical tests: One-way ANOVA with Dunnett's post hoc analysis (FIGS. 7B, 7C, 7D, 7E, 7G, 7H, 7I, and 7J); two-way ANOVA with Sidak's post hoc analysis (FIGS. 7A and 7E). **P<0.01, ***P<0.001, ****P<0.0001.

FIGS. 8A-8C show expression of Notch1, Notch2 and Notch3 on circulating Treg cells of control and COVID-19 subjects. Related to FIG. 1. FIGS. 8A-8C Flow cytometric analysis and graphical representation of Notch1 (FIG. 8A), Notch2 (FIG. 8B) and Notch3 (FIG. 8C) expression in Treg cells of control and COVID-19 subject groups. Each symbol represents one subject. Numbers in flow plots indicate percentages. Error bars indicate SEM. Statistical tests: One-way ANOVA with Dunnett's post hoc analysis.

FIGS. 9A-9N show impact of Treg cell-specific deletion of different Notch, Hippo and Wnt pathway components on poly I:C-induced lung injury. Related to FIGS. 2A-2M. A, B, Weight index (FIG. 9A) and peak weight loss (FIG. 9B) of Foxp3^{YFPCre}, Foxp3^{YFPCre}Rbpj^{Δ/Δ}, Foxp3^{YFPCre}Notch1^{Δ/Δ} Foxp3^{YFPCre}Notch2^{Δ/Δ} and Foxp3^{YFPCre}Notch3^{Δ/Δ} mice either sham-treated or treated with poly I:C, as indicated. FIG. 9C, AHR in the respective mouse groups in response to methacholine. FIGS. 9D-9F, Graphical representation of lung tissue neutrophils (FIG. 9D) and M1 (FIG. 9E) and M2 macrophages (FIG. 9F). FIGS. 9G and 9H, Flow cytometric analysis (FIG. 9G) and graphical representation (FIG. 9H) of Yap and b-Catenin in lung tissue Treg cells of Foxp3^{YFPCre} mice that were either sham-treated or treated with poly I:C. FIGS. 9I and 9J, Weight index (FIG. 9I) and peak weight loss (FIG. 9J) of Foxp3^{YFPCre}, Foxp3^{YFPCre}Yap1^{Δ/Δ}Wwtr1^{Δ/Δ}, Foxp3^{YFPCre} Ctnnb1^{Δ/Δ} and Foxp3^{YFPCre} Yap1^{Δ/Δ}Wwtr1^{Δ/Δ} Ctnnb1^{Δ/Δ} mice either sham-treated or treated with Poly I:C, as indicated. FIG. 9K, AHR in the respective mouse groups in response to methacholine. FIGS. 9L-9N, Graphical representation of lung tissue neutrophils (FIG. 9L) and M1 (FIG. 9M) and M2 (FIG. 9N) macrophages. Each symbol represents one mouse (n=5 per group). Numbers in flow plots indicate percentages. Error bars indicate SEM. Statistical tests: Two-way ANOVA with Sidak's post hoc analysis (FIGS. 9A, 9C, 9I, and 9K); One-way ANOVA with Dunnett's post hoc analysis (FIGS. 9B, 9D, 9E, 9F, 9J, 9L, 9M, and 9N); **P<0.01, ***P<0.001, ****P<0.0001.

FIGS. 10A-10G show Treg cell-specific deletion of R6ra partially protects against Poly I:C-induced lung injury. Related to FIGS. 2A-2M. FIG. 10A, BAL concentrations of IFNg, TNFa, IL-12p70, IL-1b, IFNb, IL-10, IL-1a, MCP-1, IL-27, GM-CSF, IL-17, OSM, LIF and CXCL5 in PBS or Poly I:C-treated Foxp3^{YFPCre} and Foxp3^{YFPCre}Notch4^{Δ/Δ} mice, the former also co-treated with either isotype control or anti-Notch4 mAb. FIG. 10B, Weight index (FIG. 10A) and peak weight loss of Foxp3^{YFPCre} and Foxp3YFPCreIl6ra Δ/Δ mice either sham-treated or treated with Poly I:C, as indicated. FIG. 10C, AHR in response to methacholine. FIGS. 10D and 10E, Flow cytometric analysis (FIG. 10D) and graphical representation (FIG. 10E) of Notch4 expression in lung tissue Treg cells of Foxp3^{YFPCre} and Foxp3YFPCreIl6ra Δ/Δ mice that were either sham-treated or treated with Poly I:C. FIGS. 10F-10G, Flow cytometric analysis and graphical representation of lung tissue neutrophils (FIG. 10F) and M1 (FIG. 10G) and M2 macrophages. Each symbol represents one mouse (n=5 per group). Numbers in flow plots indicate percentages. Error bars indicate SEM. Statistical tests: Two-way ANOVA with Sidak's post hoc analysis (FIGS. 10A and 10C); One-way ANOVA with Dunnett's post hoc analysis (FIGS. 10B, 10E, 10F, 10G, and 10H); **P<0.01, ****P<0.0001.

FIGS. 11A-11D show protective effect of Notch4 inhibition in influenza A H1N1 virus infection. Related to FIGS. 3A-3J. FIG. 11A, Flow cytometric analysis (FIG. 11A) and graphical representation of lung neutrophils in the indicated mouse groups. FIG. 11B, Flow cytometric analysis and graphical representation of lung M2 macrophages in the indicated mouse groups. FIG. 11C, Flow cytometric analysis and graphical representation and graphical representation of lung M1 macrophages in the indicated mouse groups. FIG. 11D, BAL concentrations of IFNg, TNFa, IL-12p70, IL-1b, IFNb, IL-10, IL-1a, MCP-1, IL-27, GM-CSF, IL-17, OSM, LIF and CXCL5 in PBS or H1N1-infected FoXP3^{YFPCre} and FoXP3YFPCreNotCh4 Δ/Δ mice, the former also co-treated with either isotype control or anti-Notch4 mAb. Each symbol represents one mouse (n=5 per group). Numbers in flow plots indicate percentages. Error bars indicate SEM. Statistical tests: One-way ANOVA with Dunnett's post hoc analysis (FIGS. 11A-11D).**P<0.01, ***P<0.001, ****P<0.0001.

FIGS. 12A-12H show characterization of lung T cell infiltrates in H1N1 infected mice. Related to FIGS. 3A-3J. A,B, Graphical representation of CD8⁺ T cell frequencies (FIG. 12A) and IFNg⁺CD8⁺ T cell frequencies and numbers (FIG. 12B) in the lungs of FoXP3^{YFPCre} and FoXP3^{YFPCre}NotCh4^{Δ/Δ} mice that were either sham-treated or infected with H1N1 virus, in the case of the FoXP3^{YFPCre} mice either alone or together with an anti-Notch4 mAb, as indicated. FIGS. 12C and 12D, Graphical representation of CD4⁺ T cell frequencies (FIG. 12C) and IFNg⁺CD4⁺ Teff cell frequencies and numbers (FIG. 12D) in the lungs. FIG. 12E, Graphical representation of IFNg⁺ Macrophage frequencies and numbers. FIG. 12F, Graphical representation of IFNg⁺ILC1 frequencies and numbers. FIG. 12G, Graphical representation of IL-17⁺CD8⁺ T cell frequencies and numbers in the lungs. FIG. 12H, Graphical representation of IL-17⁺CD4⁺ T cell frequencies and numbers in the lungs. Each symbol represents one mouse (n=5 per group). Error bars indicate SEM. Statistical tests: One-way ANOVA with Dunnett's post hoc analysis (FIGS. 12A-12H). *P<0.05, ***P<0.001, ****P<0.0001.

FIGS. 13A-1311 show validation of neutralizing anti-human Notch4 mAbs and amphiregulin91-140 blocking peptide. Related to FIGS. 4A-4J. FIG. 13A. Reactivity of different Notch4 mAbs to Notch1, Notch2, Notch3 and Notch4 as measured by ELISA. FIG. 13B, Left, Flow cytometric analysis Jagged1-Notch4^{GFP} interaction in Jurkat cells that were either sham transfected (control) or transfected with a construct encoding Notch4^{GFP} then incubated with Jagged1 in the absence or presence of the indicated mAb. FIG. 13B right, Flow cytometric analysis and graphical representation of Jagged1-PE staining of Jurkat cells that were transfected with a construct encoding Notch4^{GFP} in the absence of presence of the indicated mAb. FIG. 13C, Flow cytometric analysis and graphical representation of EGFR phosphorylation at tyrosine 1068 (pEGFR) in HEK293 cells treated with mouse amphiregulin in the presence increased concentrations of amphiregulin91-140 blocking peptide (bp) or a neutralizing anti-amphiregulin mAb, as indicated. FIG. 13D, Weight index and peak weight loss of FOXP3^{YFPCre} and FOXP3^{YFPCre}NOtCh4^{Δ/Δ} mice treated with Poly I:C, either alone or together with anti-amphiregulin neutralizing mAb. FIG. 13E, Frequencies of neutrophils and M1 and M2 macrophages in lung tissues. FIG. 13F, Serum tetramethylrhodamine isothiocyanate (TRITC) dextran, measured as relative fluorescent units (RFU), in Poly I:C+ anti-Amphiregulin mAb-treated FOXP3^{YFPCre} or FOXP3^{YFPCre}NOtCh4^{Δ/Δ} mice at 1-hour post intra-tracheal instillation. FIG. 13G, Frequencies of neutrophils and M1 and M2 macrophages in lung tissues from FOXP3^{YFPCre} mice either sham treated or treated with Poly I:C, either alone or together with amphiregulin bp. FIG. 13H, frequencies of M1 and M2 macrophages in co-cultures of Poly I:C-treated lung macrophages incubated either alone, with amphiregulin, Treg cells from Poly I:C-treated FOXP3^{YFPCre} mice or the combination thereof. Each symbol represents one mouse (n=5-10 per group). Numbers in flow plots indicate percentages. Error bars indicate SEM. Statistical tests: One-way ANOVA with Dunnett's post hoc analysis (FIGS. 13D-13F); two-way ANOVA with Sidak's post hoc analysis (FIG. 13D); Student's two tailed t-test (FIG. 13G). **P<0.01, ****P<0.0001).

FIGS. 14A-14C show anti-human Notch4 mAb (4111) treatment protects humanized mice against lethal H1N1 infection. FIGS. 14A and 14B. Body weight index (FIG. 14A) and Body weight index at peak weight loss (FIG. 14B) of humanized mice infected with a lethal dose of H1N1 influenza virus that were treated with either an isotype control IgG mAb (n=6) or the neutralizing anti-human Notch4 mAb 4H1 (n=8). FIG. 14C, Survival curve of the humanized mouse groups shown in FIGS. 14A and 14B. Each symbol represents one mouse. Error bars indicate SEM. Statistical tests: two-way ANOVA with Sidak's post hoc analysis (FIG. 14A), Student's t-test (FIG. 14B). *P<0.05, **P<0.01, ****P<0.0001.

### DETAILED DESCRIPTION

Provided herein are methods and compositions comprising antibodies or antigen-binding fragments that bind to Notch4 and inhibit Notch4 activity in a cell, e.g., a Treg. Such antibodies or antigen-binding fragments can be used in the treatment of a disease or disorder associated with an increased level of Notch4 (e.g., coronavirus), or other situations where suppression of Notch4 is desirable.

### Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies can be from any source, but primate (human and non-human primate) and primatized are preferred

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain such that one or more CDR regions are positioned in a conformation suitable for an antigen binding site. For example, the sequence can include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site, e.g., a structure that preferentially interacts with a Notch4 protein.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds or a linker. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin can be of types kappa or lambda. In one embodiment, the antibody is glycosylated.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified to include a sufficient number of human framework amino acid positions such that the immunoglobulin variable region does not elicit an immunogenic response in a normal human (i.e., a subject that does not have an immune disorder). Descriptions of “humanized” immunoglobulins include, for example, U.S. Pat. Nos. 6,407,213 and 5,693,762.

As used herein, “binding affinity” refers to the apparent association constant or Ka. The Ka is the reciprocal of the dissociation constant (Kd). A binding protein can, for example, have a binding affinity of at least 10⁵, 10⁶, 10⁷, 10⁸, 10⁹, 10¹⁰ and 10¹¹ M⁻¹ for a particular target molecule, e.g., Notch4. Higher affinity binding of a binding protein to a first target (e.g., Notch4) relative to a second target (e.g., a non-specific protein) can be indicated by a higher Ka (or a smaller numerical value Kd) for binding the first target than the Ka (or numerical value Kd) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 105 fold.

In some embodiment, an antibody reagent (e.g., an antibody or antigen-binding domain thereof) specifically binds to Notch4 present on the cell-surface with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less and binds to that target at least 100×, or 1000×, or 10,000× and preferably more strongly than it binds to an off-target or distinct cell-surface marker or protein. The person of ordinary skill in the art can determine appropriate conditions under which the polypeptide agents described herein selectively bind Notch4 using any suitable methods, such as titration of the antibody reagent in a suitable cell binding assay. Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂ at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/Ka)+[Free]).

It is not always necessary to make an exact determination of Ka, though, since sometimes it is sufficient to obtain a quantitative measurement of affinity, e.g., determined using a method such as ELISA or FACS analysis, is proportional to Ka, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively binds the antigen. A commonly used antigen-binding fragment includes the V_{H} and V₂ domains of an antibody, which can be joined either via part of the constant domains of the heavy and light chains of an antibody, or, alternatively, by a linker, such as a peptide linker. Non-conventional antibodies, such as camelid and short antibodies have only 2 heavy chain sequences, denoted, for example V_{HH}. These can be used in a manner analogous to V_{H}/VL-containing antigen-binding fragments. Non-limiting examples of antibody fragments encompassed by the term antigen-binding fragment include: (i) a Fab fragment, having V_{L}, C_{L}, V_{H} and CH1 domains; (ii) a Fab′ fragment, which is a Fab fragment having one or more cysteine residues at the C-terminus of the CH1 domain; (iii) an Fd fragment having V_{H} and C_{H}1 domains; (iv) a Fd′ fragment having V_{H} and C_{H}1 domains and one or more cysteine residues at the C-terminus of the CH1 domain; (v) an Fv fragment having the V_{L} and V_{H} domains of a single arm of an antibody; (vi) a dAb fragment (Ward et al., Nature 341, 544-546 (1989)) which consists of a V_{H} domain; (vii) F(ab′)₂ fragments, a bivalent fragment including two Fab′ fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); and (x) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870). The molecules of Fv, scFv or diabody can be stabilized by incorporating disulfide bridges linking the VH and VL domains. Minibodies comprising a scFv fragment linked to a CH3 domain can also be obtained. Other examples of binding fragments are Fab′, which differs from Fab fragments by the addition of some residues at the carboxyl terminus of the CH1 domain of the heavy chain, including one or more cysteines of the hinge region of the antibody, and Fab′-SH, which is a Fab′ fragment in which the cysteine residue(s) of the constant domains carries a free thiol group.

In some embodiments of any one of the aspects, the antibody or the antigen binding fragment is an isolated antibody or antigen binding fragment. An “isolated antibody” is intended to refer to an antibody that is substantially free of other antibodies having different antigenic specificities (e.g., an isolated antibody that specifically binds to Notch4 is substantially free of antibodies that specifically bind antigens other than Notch4). An isolated antibody that specifically binds to Notch4 can, however, have cross-reactivity to other antigens, such as to Notch4 molecules from other species. Moreover, an isolated antibody can be substantially free of other cellular material and/or chemicals.

As used herein, the term “specificity” refers to the number of different types of antigens or antigenic determinants to which an antibody or antibody fragment thereof as described herein can bind. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof: the lesser the value of the K_{D}, the stronger the binding strength between an antigenic determinant and the antigen-binding molecule. Alternatively, the affinity can also be expressed as the affinity constant (K_{A}), which is 1/K_{D}). Accordingly, an antibody or antibody fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10000 times or more better than the affinity with which said amino acid sequence or polypeptide binds to another target or polypeptide. Antibody affinities can be determined, for example, by a surface plasmon resonance based assay (such as the BIACORE assay described in PCT Application Publication No. WO2005/012359); enzyme-linked immunosorbent assay (ELISA); and competition assays (e.g., RIA's), for example.

As used herein, “avidity” is a measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D}) of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (K_{A}) of 10⁵ to 10¹² liter/moles or more, such as 10⁷ to 10¹² liter/moles or 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (MA), enzyme immunoassays (EIA) and sandwich competition assays, and the different variants thereof known in the art; as well as other techniques as mentioned herein.

Accordingly, as used herein, “selectively binds” or “specifically binds” refers to the ability of an antibody or antigen-binding fragment thereof as described herein to bind to a target, such as Notch4, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, the term “selectively inhibits” means that an agent, such as a bispecific antibody agent, inhibits, as that term is used herein, the association of a first ligand-receptor pair (e.g., Notch4 and its cognate ligand) but does not substantially inhibit the association of a relevant second ligand-receptor pair.

The term “universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. 196:910-917 (1987). The Kabat database is now also maintained on the world wide web. The compositions and methods described herein provide for the use of a single framework, or a set of such frameworks, which have been found to permit the derivation of virtually any binding specificity though variation in the hypervariable regions alone. The universal framework can be a V_{L} framework (V_{λ} or V_{κ}), such as a framework that comprises the framework amino acid sequences encoded by the human germline DPK1, DPK2, DPK3, DPK4, DPK5, DPK6, DPK7, DPK8, DPK9, DPKIO, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline J_{κ}1, J_{κ}2, J_{κ}3, J_{κ}4, or J_{κ}5 immunoglobulin gene segments. In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segments. In some embodiments, the V_{H} framework can further comprise the framework amino acid sequence encoded by the human germline J_{H}1, J_{H}2, J_{H}3, J_{H}4, J_{H}4b, J_{H}5 or J_{H}6 immunoglobulin gene segments.

An “Fv” fragment is an antibody fragment which contains a complete antigen recognition and binding site. This region consists of a dimer of one heavy and one light chain variable domain in tight association, which can be covalent in nature, for example in a single-chain Fv or scFv (see below). It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. For the methods and compositions described herein, the amino acid positions assigned to CDRs and FRs can be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

A “Fab” of “Fab fragment” fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which permits the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (VH-CHI-VH-CH1) which, together with complementary light chain polypeptides, form a pair of antigen binding regions. Linear antibodies can be bispecific or monospecific.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein in relation to antibody domains, “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will ideally comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). The constant region, can if desired, include one or more modifications that modify or disrupt interaction of the human or humanized antibody with an Fc receptor, as described herein. Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder associated with an increased level of Notch4. A subject can be male or female. In addition, a subject can be of any age, including a neonate, a toddler, a child, a pre-adolescent, an adolescent, or an adult or geriatric subject.

A subject can be one who has been previously diagnosed with or identified as suffering from or having a condition in need of treatment (e.g., a disease or disorder associated with an increased level of Notch4) or one or more complications related to such disease or disorder. Alternatively, a subject can also be one who has not been previously diagnosed as having a disease or disorder or one or more complications related thereto. For example, a subject can be one who exhibits one or more risk such diseases or disorders.

A “subject in need” of treatment for a particular condition can be a subject having that condition, diagnosed as having that condition, or at risk of developing that condition.

The term “therapeutically effective amount” refers to an amount of an inhibitor, e.g., an antibody as described herein, that is effective to inhibit Notch4 activity, thereby treating a disease or disorder associated with an increased level of Notch4, or reducing or relieving a symptom thereof. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the terms “treat,” “treatment,” “treating,” or “amelioration” refer to therapeutic treatments, wherein the object is to reverse, alleviate, ameliorate, inhibit, slow down or stop the progression or severity of at least one symptom of a disease or disorder associated with an increased level of Notch4. The term “treating” includes reducing or alleviating at least one adverse effect or symptom of a disease or disorder. Treatment is generally “effective” if one or more symptoms or clinical markers of the disease or disorder being treated is reduced. Alternatively, treatment is “effective” if the progression of the disease or disorder is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total). The term “treatment” of a disease also includes providing relief from the symptoms or side-effects of the disease (including palliative treatment).

The term “prophylactically effective amount” refers to an amount of a Notch4 antibody or antigen-binding fragment thereof which is effective, at dosages and for periods of time necessary, to achieve the desired prophylactic result, e.g., the amount of a Notch4 antibody or fragment thereof to delay the onset of or reduce the intensity of at least one symptom of a disease or disorder associated with an increased level of Notch4. Typically, since a prophylactic dose of a Notch4 antibody is administered to a subject prior to onset of a disease or disorder, or at an early stage of a disease or disorder, the dose necessary for a prophylactically effective amount can be less than the therapeutically effective amount. A prophylactically effective amount of Notch4 antibody or fragment thereof is also one in which any toxic or detrimental effects of the compound are outweighed by the beneficial effects.

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The “percent identity” or “percent homology” of two polynucleotide or two polypeptide sequences can be determined by comparing the sequences using the GAP computer program (a part of the GCG Wisconsin Package, version 10.3 (Accelrys, San Diego, Calif.)) using its default parameters.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, the degree of complementarity, when optimally aligned using a suitable alignment algorithm, is about or more than about 50%, 60%, 75%, 80%, 85%, 90%, 95%, 97.5%, 99%), or more. Optimal alignment can be determined with the use of any suitable algorithm for aligning sequences. Exemplary algorithms for determining optimal alignment include, but are not limited to, the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Disease and Disorders Associated with Increased Notch4 Levels

Various aspects of this invention relate to a method of treating a disease or disorder associated with an increased level of Notch4. As used herein, “a disease or disorder associated with increased levels of Notch4” refers to a disease or disorder that is results from, or is caused, induced, provoked, worsened, or accompanied by an increased level of Notch4, as compared to an appropriate control. As used herein, an “reference level” refers to a Notch4 level a normal, otherwise healthy cell population or tissue. In some embodiments, the level of Notch4 is increased at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

In some embodiments, the disease or disorder associated with increased level of Notch4 is associated with airway inflammation, e.g., chronic airway inflammation. In one embodiment, the disease or disorder associated with an increased level of Notch4 is asthma or an allergic disease. As used herein, an “asthma” refers to a disease characterized by inflammation in the airways of the lungs, reversible airways obstructions, bronchospasms, wheezing, coughing, tightness of the chest, and shortness of breath. Asthma is thought to be caused by environmental and genetic factors, include, but not limited to exposure to air pollutants and allergens, aspirin and beta blockers, and a family history of asthma.

Asthma is classified by the frequency of symptoms, the severity of symptoms, forced expiratory volume in one second (FEV1), and peak expiratory flow rate. Asthma can further be classified based on the subject's response to a medication, e.g., atopic or non-atopic, wherein atropic refers to a predisposition towards developing a type 1 hypersensitivity.

In various embodiments, the asthma is allergic asthma (e.g., induced by exposure to allergens), asthma without allergies (e.g., induced by an upper respiratory infection, such as a cold, flu, or rhinovirus), aspirin exacerbated respiratory disease (e.g., induced by the intake of aspirin), exercised-induced asthma, cough variant (e.g., characterized by a dry, hacking cough), or occupational asthma (e.g., induced by an irritant a subject is exposed to on a job, for example, a fire fighter is exposed to smoke, and can experience smoke-inhalation, while performing their job). A skilled clinician can identify a type of asthma a subject has, or is at risk of having (e.g., a fire fighter would be at risk of having occupational asthma), using standard techniques.

As used herein, an “allergic disease” is a disease that is characterized by an immune system response to an otherwise harmless substance in the environment. For example, when a subject who has an allergic disease is exposed to common environmental substances the subject's B lymphocytes produce specific antibodies against that substance, resulting in an immune response. Exemplary substances that, e.g., can cause an allergic disease include dust mites, pollen (e.g., from plants, trees, flowers, or grass), animal dander (e.g., from domestic or farm animals), mold, food (e.g., tree nuts, peanuts, shellfish, fish, milk, eggs, or wheat), and latex. A child whose parent, or parents, have allergies are at an increased risk of developing an allergic disease. The specific cause of an allergic diseases (e.g., what the allergen is) can be identified by a skilled clinician using common techniques, e.g., skin prick tests and radioallergosorbent tests.

In one embodiment, the allergic disease is allergic rhinitis, sinusitis, otitis media, atopic dermatitis (e.g., eczema), urticaria, angioedema, and anaphylaxis.

In one embodiment, the disease or disorder associated with increased level of Notch4 is a disease or disorder associated with pulmonary viral infections (e.g., diseases or disorders associated with influenza virus infections, coronavirus infectious disease, etc.). In one embodiment, the disease or disorder associated with an increased level of Notch4 is a coronavirus infectious disease. Coronaviruses belong to the subfamily Coronavirinae in the family Coronaviridae and are named for the crown-like spikes on their surface. There are four main sub-groupings of coronaviruses, known as alpha, beta, gamma, and delta. Coronaviruses viruses typically affect the respiratory tracts of birds and mammals, including humans. The most recent data suggest that there are 7 coronaviruses that are capable of infecting humans.

Globally, people are commonly infected with human coronaviruses 229E, NL63, OC43, and HKU1, resulting in the common cold. Infection by these coronaviruses most often occur during the winter months and early spring. At times, a coronavirus that previously infected an animal will infect a human, resulting in a new human coronavirus. These new viruses are more rare, but can result in a more severe infection. Three recent examples of this are SARS-CoV-2 (the novel coronavirus that causes coronavirus disease 2019 (COVID-19), SARS-CoV (the beta coronavirus that causes severe acute respiratory syndrome, or SARS), and MERS-CoV (the beta coronavirus that causes Middle East Respiratory Syndrome, or MERS).

Symptoms associated with a typical coronavirus infectious disease (e.g., the common cold) include runny nose, headache, cough, fever, and sore throat. There is currently no cure for the common cold; treatments typically include self-care and over-the-counter medications to manage symptoms.

Symptoms associated with an infection by SARS-CoV-2, resulting in COVID-19, include fever, chills, persistent dry cough, shortness of breath, sore throat, headache, loss of taste or smell, and gastrointestinal distress. The development of a serious illness and/or a poor outcome from COVID-19 is most commonly observed in subjects over the age of 65, having chronic lung disease, serious heart conditions, severe obesity, a compromised immune system, or diabetes, and living in a nursing home or care facility. COVID-19 is rarely observed in subjects under the age of 18.

In one embodiment, the disease or disorder associated with an increased level of Notch4 is acute respiratory distress syndrome (ARDS). ARDS is a life threatening syndrome that occurs when fluid builds up in the alveoli in your lungs. The fluid keeps your lungs from filling with enough air, resulting in a markedly reduced level of oxygen reaching your bloodstream, depriving your organs of the oxygen. ARDS typically occurs in people who are already critically ill or who have significant injuries. Symptoms of ARDS usually develops within a few hours to a few days after the precipitating injury or infection. The risk of death associated with ARDS increases with age (those 60 years of age and older are at a greater risk of death) and severity of illness. Of the people who do survive ARDS, some recover completely while others experience lasting damage to their lungs.

The most common underlying causes and risk factors of ARDS include sepsis (i.e., a serious and widespread infection of the bloodstream); inhalation of harmful substances or aspirating vomit or near-drowning episodes; severe pneumonia, e.g., pneumonia that affects all five lobes of the lungs; head, chest or other major injury, e.g., that directly damage the lungs or the portion of the brain that controls breathing; pancreatitis; massive blood transfusions; and burns. ARDS can be diagnosed by a skilled clinician by determining if a subject exhibit at least one symptom of ARDS, e.g., shortness of breath, labored and unusually rapid breathing, low blood pressure, and confusion and extreme tiredness.

Subjects with ARDS are at a greater risk of developing blood clots, collapsed lung, secondary infections, and pulmonary fibrosis.

Further provided herein are methods for preventing a disease or disorder associated with an increased level of Notch4 in a subject at risk of developing such disease or disorder. For example, an anti-Notch4 antibody described herein can be administered to a subject that is at risk of developing a disease or disorder associated with an increased level of Notch4. In one embodiment, the subject is identified as being at risk prior to administering an anti-Notch4 antibody.

Notch4

Neurogenic locus notch homolog 4, also known as “Notch4” refers to a type I transmembrane protein, which is a member of a family that share structural characteristics, including an extracellular domain consisting of multiple epidermal growth factor-like (EGF) repeats, and an intracellular domain consisting of multiple different domain. Notch4 sequences are known for a number of species, e.g., human Notch4 (NCBI Gene ID: 4855) polypeptide (e.g., NCBI Ref Seq NP_004548.3) and mRNA (e.g., NCBI Ref Seq NM_004557.3). Notch4 can refer to human Notch4, including naturally occurring variants, molecules, and alleles thereof. Notch4 refers to the mammalian Notch4 of, e.g., mouse, rat, rabbit, dog, cat, cow, horse, pig, and the like. The nucleic sequence of SEQ ID NO: 1 comprises a nucleic sequence which encodes Notch4.

The Notch signaling pathway is an evolutionarily conserved intercellular signaling pathway that regulates interactions between physically adjacent cells. Notch signaling regulates multiple cell fate decisions; each Notch family member plays a role in a variety of developmental processes. In mammals, the Notch family is composed of four Notch receptors (Notch1-Notch4) and five ligands [Delta-like ligand 1 (DLL1), DLL3, DLL4, Jagged(Jag)1 and Jag2]. Upon binding to Jagged or Delta-like ligands on an adjacent cell, two sequential proteolytic events release the intracellular domain of Notch (NICD) allowing its translocation to the nucleus. There the NICD converts the DNA binding factor RBP-J from a transcriptional repressor to a transcriptional activator through MAML1-MAML3 binding 1.

The notch protein is cleaved in the trans-Golgi network, and then presented on the cell surface as a heterodimer. The protein functions as a receptor for membrane bound ligands, and may play a role in vascular, renal, and hepatic development.

SEQ ID NO: 1 contains a nucleic acid sequence that
encodes Notch 4.
(SEQ ID NO: 1)
a tgcagccccc ttcactgctg ctgctgctgc tgctgctgct
gctgctatgt gtctcagtgg tcagacccag agggctgctg tgtgggagtt tcccagaacc
ctgtgccaat ggaggcacct gcctgagcct gtctctggga caagggacct gccagtgtgc
ccctggcttc ctgggtgaga cgtgccagtt tcctgacccc tgccagaacg cccagctctg
ccaaaatgga ggcagctgcc aagccctgct tcccgctccc ctagggctcc ccagctctcc
ctctccattg acacccagct tcttgtgcac ttgcctccct ggcttcactg gtgagagatg
ccaggccaag cttgaagacc cttgtcctcc ctccttctgt tccaaaaggg gccgctgcca
catccaggcc tcgggccgcc cacagtgctc ctgcatgcct ggatggacag gtgagcagtg
ccagcttcgg gacttctgtt cagccaaccc atgtgttaat ggaggggtgt gtctggccac
atacccccag atccagtgcc actgcccacc gggcttcgag ggccatgcct gtgaacgtga
tgtcaacgag tgcttccagg acccaggacc ctgccccaaa ggcacctcct gccataacac
cctgggctcc ttccagtgcc tctgccctgt ggggcaggag ggtccacgtt gtgagctgcg
ggcaggaccc tgccctccta ggggctgttc gaatgggggc acctgccagc tgatgccaga
gaaagactcc acctttcacc tctgcctctg tcccccaggt ttcataggcc cagactgtga
ggtgaatcca gacaactgtg tcagccacca gtgtcagaat gggggcactt gccaggatgg
gctggacacc tacacctgcc tctgcccaga aacctggaca ggctgggact gctccgaaga
tgtggatgag tgtgagaccc agggtccccc tcactgcaga aacgggggca cctgccagaa
ctctgctggt agctttcact gcgtgtgtgt gagtggctgg ggcggcacaa gctgtgagga
gaacctggat gactgtattg ctgccacctg tgccccggga tccacctgca ttgaccgggt
gggctctttc tcctgcctct gcccacctgg acgcacagga ctcctgtgcc acttggaaga
catgtgtctg agccagccgt gccatgggga tgcccaatgc agcaccaacc ccctcacagg
ctccacactc tgcctgtgtc agcctggcta ttcggggccc acctgccacc aggacctgga
cgagtgtctg atggcccagc aaggcccaag tccctgtgaa catggcggtt cctgcctcaa
cactcctggc tccttcaact gcctctgtcc acctggctac acaggctccc gttgtgaggc
tgatcacaat gagtgcctct cccagccctg ccacccagga agcacctgtc tggacctact
tgccaccttc cactgcctct gcccgccagg cttagaaggg cagctctgtg aggtggagac
caacgagtgt gcctcagctc cctgcctgaa ccacgcggat tgccatgacc tgctcaacgg
cttccagtgc atctgcctgc ctggattctc cggcacccga tgtgaggagg atatcgatga
gtgcagaagc tctccctgtg ccaatggtgg gcagtgccag gaccagcctg gagccttcca
ctgcaagtgt ctcccaggct ttgaagggcc acgctgtcaa acagaggtgg atgagtgcct
gagtgaccca tgtcccgttg gagccagctg ccttgatctt ccaggagcct tcttttgcct
ctgcccctct ggtttcacag gccagctctg tgaggttccc ctgtgtgctc ccaacctgtg
ccagcccaag cagatatgta aggaccagaa agacaaggcc aactgcctct gtcctgatgg
aagccctggc tgtgccccac ctgaggacaa ctgcacctgc caccacgggc actgccagag
atcctcatgt gtgtgtgacg tgggttggac ggggccagag tgtgaggcag agctaggggg
ctgcatctct gcaccctgtg cccatggggg gacctgctac ccccagccct ctggctacaa
ctgcacctgc cctacaggct acacaggacc cacctgtagt gaggagatga cagcttgtca
ctcagggcca tgtctcaatg goggctcctg caaccctagc cctggaggct actactgcac
ctgccctcca agccacacag ggccccagtg ccaaaccagc actgactact gtgtgtctgc
cccgtgcttc aatgggggta cctgtgtgaa caggcctggc accttctcct gcctctgtgc
catgggcttc cagggcccgc gctgtgaggg aaagctccgc cccagctgtg cagacagccc
ctgtaggaat agggcaacct gccaggacag ccctcagggt ccccgctgcc tctgccccac
tggctacacc ggaggcagct gccagactct gatggactta tgtgcccaga agccctgccc
acgcaattcc cactgcctcc agactgggcc ctccttccac tgcttgtgcc tccagggatg
gaccgggcct ctctgcaacc ttccactgtc ctcctgccag aaggctgcac tgagccaagg
catagacgtc tcttcccttt gccacaatgg aggcctctgt gtcgacagcg gcccctccta
tttctgccac tgcccccctg gattccaagg cagcctgtgc caggatcacg tgaacccatg
tgagtccagg ccttgccaga acggggccac ctgcatggcc cagcccagtg ggtatctctg
ccagtgtgcc ccaggctacg atggacagaa ctgctcaaag gaactcgatg cttgtcagtc
ccaaccctgt cacaaccatg gaacctgtac toccaaacct ggaggattcc actgtgcctg
ccctccaggc tttgtggggc tacgctgtga gggagacgtg gacgagtgtc tggaccagcc
ctgccacccc acaggcactg cagcctgcca ctctctggcc aatgccttct actgccagtg
tctgcctgga cacacaggcc agtggtgtga ggtggagata gacccctgcc acagccaacc
ctgctttcat ggagggacct gtgaggccac agcaggatca cccctgggtt tcatctgcca
ctgccccaag ggttttgaag gccccacctg cagccacagg gccccttcct gcggcttcca
tcactgccac cacggaggcc tgtgtctgcc ctcccctaag ccaggcttcc caccacgctg
tgcctgcctc agtggctatg ggggtcctga ctgcctgacc ccaccagctc ctaaaggctg
tggccctccc tccccatgcc tatacaatgg cagctgctca gagaccacgg gcttgggggg
cccaggcttt cgatgctcct gccctcacag ctctccaggg ccccggtgtc agaaacccgg
agccaagggg tgtgagggca gaagtggaga tggggcctgc gatgctggct gcagtggccc
gggaggaaac tgggatggag gggactgctc tctgggagtc ccagacccct ggaagggctg
cccctcccac tctcggtgct ggcttctctt ccgggacggg cagtgccacc cacagtgtga
ctctgaagag tgtctgtttg atggctacga ctgtgagacc cctccagcct gcactccagc
ctatgaccag tactgccatg atcacttcca caacgggcac tgtgagaaag gctgcaacac
tgcagagtgt ggctgggatg gaggtgactg caggcctgaa gatggggacc cagagtgggg
gccctccctg gccctgctgg tggtactgag ccccccagcc ctagaccagc agctgtttgc
cctggcccgg gtgctgtccc tgactctgag ggtaggactc tgggtaagga aggatcgtga
tggcagggac atggtgtacc cctatcctgg ggcccgggct gaagaaaagc taggaggaac
tcgggacccc acctatcagg agagagcagc ccctcaaacg cagcccctgg gcaaggagac
cgactccctc agtgctgggt ttgtggtggt catgggtgtg gatttgtccc gctgtggccc
tgaccacccg gcatcccgct gtccctggga ccctgggctt ctactccgct tccttgctgc
gatggctgca gtgggagccc tggagcccct gctgcctgga ccactgctgg ctgtccaccc
tcatgcaggg accgcacccc ctgccaacca gottccctgg cctgtgctgt gctccccagt
ggccggggtg attctcctgg ccctaggggc tcttctcgtc ctccagctca tccggcgtcg
acgccgagag catggagctc tctggctgcc ccctggtttc actcgacggc ctcggactca
gtcagctccc caccgacgcc ggcccccact aggcgaggac agcattggtc tcaaggcact
gaagccaaag gcagaagttg atgaggatgg agttgtgatg tgctcaggcc ctgaggaggg
agaggaggtg ggccaggctg aagaaacagg cccaccctcc acgtgccagc tctggtctct
gagtggtggc tgtggggcgc tccctcaggc agccatgcta actcctcccc aggaatctga
gatggaagcc cctgacctgg acacccgtgg acctgatggg gtgacacccc tgatgtcagc
agtttgctgt ggggaagtac agtccgggac cttccaaggg gcatggttgg gatgtcctga
gccctgggaa cctctgctgg atggaggggc ctgtccccag gctcacaccg tgggcactgg
ggagaccccc ctgcacctgg ctgcccgatt ctcccggcca accgctgccc gccgcctcct
tgaggctgga gccaacccca accagccaga ccgggcaggg cgcacacccc ttcatgctgc
tgtggctgct gatgctcggg aggtctgcca gottctgctc cgtagcagac aaactgcagt
ggacgctcgc acagaggacg ggaccacacc cttgatgctg gctgccaggc tggcggtgga
agacctggtt gaagaactga ttgcagccca agcagacgtg ggggccagag ataaatgggg
gaaaactgcg ctgcactggg ctgctgccgt gaacaacgcc cgagccgccc gctcgcttct
ccaggccgga gccgataaag atgcccagga caacagggag cagacgccgc tattcctggc
ggcgcgggaa ggagcggtgg aagtagccca gctactgctg gggctggggg cagcccgaga
gctgcgggac caggctgggc tagcgccggc ggacgtcgct caccaacgta accactggga
tctgctgacg ctgctggaag gggctgggcc accagaggcc cgtcacaaag ccacgccggg
ccgcgaggct gggcccttcc cgcgcgcacg gacggtgtca gtaagcgtgc ccccgcatgg
gggcggggct ctgccgcgct gccggacgct gtcagccgga gcaggccctc gtgggggcgg
agcttgtctg caggctcgga cttggtccgt agacttggct gcgcgggggg gcggggccta
ttctcattgc cggagcctct cgggagtagg agcaggagga ggcccgaccc ctcgcggccg
taggttttct gcaggcatgc gogggcctcg gcccaaccct gcgataatgc gaggaagata
cggagtggct gccgggcgcg gaggcagggt ctcaacggat gactggccct gtgattgggt
ggccctggga gottgcggtt ctgcctccaa cattccgate ccgcctcctt g

In one embodiment, an agent that “reduces or inhibits Notch4 activity” is an antibody or antigen-binding fragment thereof as described herein that reduces interaction of Notch4 with one or more of its cognate receptors or ligands.

In one embodiment, upon administration, the antibody or antibody reagent inhibits the presence, amount, activity and/or level of Notch4 in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control would be the presence, amount, activity and/or level of Notch4 in a substantially identical cell that is not administered an agent described herein.

In one embodiment, inhibiting Notch4 inhibits the differentiation of a Notch4-expressing Treg cell into a disease-promoting Th cell.

In one embodiment, inhibiting Notch4 inhibits the transcription, or the translation of Notch4 in the cell.

In one embodiment, inhibiting Notch4 inhibits the activity or alters the activity (e.g., such that the activity no longer occurs, or occurs at a reduced rate) of Notch4 in the cell (e.g., Notch4's expression).

In one embodiment of any aspect, Notch4 is inhibited on T regulatory cells. In one embodiment, an agent that inhibits Notch4 promotes programmed cell death, e.g., kill, the cell that expresses Notch4, for example, a T reg cell. To determine is an agent is effective at inhibiting Notch4, mRNA and protein levels of a given target (e.g., Notch4) can be assessed using RT-PCR and western-blotting, respectively. Biological assays that detect the activity of Notch4 (e.g., Notch reporters that measure the binding of the Notch receptor and ligand) can be used to assess if programmed cell death has occurred. Alternatively, immunofluorescence detection using antibodies specific to Notch4 in combination with cell death markers (e.g., Caspase) can be used to determine if cell death has occurred following administration of an agent.

In one embodiment, the level and/or activity of Notch4 by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of Notch4 prior to administration of the agent, or the level and/or activity of Notch4 in a population of cells that was not in contact with the agent.

### Antibodies

Provided herein are exemplary antibodies and antigen-binding fragments that bind to Notch4. In some embodiments, such antibodies or antigen-binding fragments can inhibit or reduce Notch4 activity or interaction of Notch4 with its cognate ligand. For example, the antibody or antigen-binding fragment binds to or sterically blocks the ligand binding site on Notch4.

The antibody or antigen-binding fragment described herein can be in form of a full antibody or a fragment thereof, e.g., a Fab, F(ab′)2, Fv, or a single chain Fv fragment (scFv). For example, the antibody or antigen-binding fragment can be selected from the group consisting of Fv, Fab, Fab′, F(ab)2, F(ab′)2, single-chain antibody, single-chain antibody (scFV), sc(Fv)2, monovalent antibody lacking hinge region, whole antibody, disulfide-stabilized Fv (dsFv), diabody dAb, a bivalent or bispecific antibody, a nanobody, a monoclonal antibody, a humanized antibody, a human antibody, a recombinant antibody, or a chimeric antibody.

In some embodiments of any one of the aspects, the antibody or antigen-binding fragment thereof comprises a heavy chain variable region (V_{H}) comprising a complementarity determining region 1 (CDR_H1) sequence that differs by no more than four, preferably no more than three, more preferably no more than two, most preferably no more than one amino acid from the amino acid sequence selected from the group consisting of: SEQ ID NOs: 16, 22, 28, 34.

In some embodiments of any one of the aspects, the antibody or antigen-binding fragment thereof comprises a heavy chain variable region comprising a complementarity determining region 2 (CDR_H2) sequence that differs by no more than four, preferably no more than three, more preferably no more than two, most preferably no more than one amino acid from the amino acid sequence selected from the group consisting of: SEQ ID NOs: 17, 23, 29, 35.

In some embodiments of any one of the aspects, the antibody or antigen-binding fragment thereof comprises a heavy chain variable region comprising a complementarity determining region 3 (CDR_H3) sequence that differs by no more than four, preferably no more than three, more preferably no more than two, most preferably no more than one amino acid from the amino acid sequence selected from the group consisting of: SEQ ID NOs: 18, 24, 30, 36.

In some embodiments of any one of the aspects, the antibody or antigen-binding fragment thereof comprises a light chain variable region (V_{L}) comprising a complementarity determining region 1 (CDR_L1) sequence that differs by no more than four, preferably no more than three, more preferably no more than two, most preferably no more than one amino acid from the amino acid sequence selected from the group consisting of: SEQ ID NOs: 19, 25, 31, 37.

In some embodiments of any one of the aspects, the antibody or antigen-binding fragment thereof comprises a light chain variable region comprising a complementarity determining region 2 (CDR_L2) sequence that differs by no more than four, preferably no more than three, more preferably no more than two, most preferably no more than one amino acid from the amino acid sequence selected from the group consisting of: SEQ ID NOs: 20, 26, 32, 38.

In some embodiments of any one of the aspects, the antibody or antigen-binding fragment thereof comprises a light chain variable region comprising a complementarity determining region 3 (CDR_L3) sequence that differs by no more than four, preferably no more than three, more preferably no more than two, most preferably no more than one amino acid from the amino acid sequence selected from the group consisting of: SEQ ID NOs: 21, 27, 33, 39.

In some embodiments of any one of the aspects, the antibody or antigen-binding fragment thereof comprises a heavy chain variable region comprising an amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 2, 4, and 6. For example, the V_{H} region comprises an amino acid sequence having at least 90%, 91%, 92%, 93%, 94%, 95%, 96%, 97%, 98% or 99% identity to an amino acid sequence selected from the group consisting of SEQ ID NOs: 2, 4, and 6. In some embodiments, the V_{H} region comprises an amino acid sequence having at least 95%, 96%, 97%, 98% or 99% identity to an amino acid sequence selected from the group consisting of SEQ ID NOs: 2, 4, and 6.

In some embodiments of any one of the aspects, the antibody or antigen-binding fragment thereof comprises a heavy chain variable region comprising a nucleotide sequence having at least 85%, 86%, 87%, 88%, 89%, 90%, 91%, 92%, 93%, 94%, 95%, 96%, 97%, 98% or 99% identity to an nucleotide sequence selected from the group consisting of SEQ ID NOs: 3, 5, and 7. For example, the V_{H} region comprises an nucleotide sequence having at least 90%, 91%, 92%, 93%, 94%, 95%, 96%, 97%, 98% or 99% identity to a nucleotide sequence selected from the group consisting of SEQ ID NOs: 3, 5, and 7. In some embodiments, the V_{H} region comprises a nucleotide sequence having at least 95%, 96%, 97%, 98% or 99% identity to a nucleotide sequence selected from the group consisting of SEQ ID NOs: 3, 5, and 7.

In some embodiments of any one of the aspects, the antibody or antigen-binding fragment thereof comprises a light chain variable region comprising an amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 8, 10, 12 and 14. For example, the V_{L} region comprises an amino acid sequence having at least 90%, 91%, 92%, 93%, 94%, 95%, 96%, 97%, 98% or 99% identity to an amino acid sequence selected from the group consisting of SEQ ID NOs: 8, 10, 12 and 14. In some embodiments, the V_{L} region comprises an amino acid sequence having at least 95%, 96%, 97%, 98% or 99% identity to an amino acid sequence selected from the group consisting of SEQ ID NOs: 8, 10, 12 and 14.

In some embodiments of any one of the aspects, the antibody or antigen-binding fragment thereof comprises a light chain variable region comprising a nucleotide sequence having at least 85%, 86%, 87%, 88%, 89%, 90%, 91%, 92%, 93%, 94%, 95%, 96%, 97%, 98% or 99% identity to an nucleotide sequence selected from the group consisting of SEQ ID NOs: 9, 11, 13, and 15. For example, the V_{L} region comprises an nucleotide sequence having at least 90%, 91%, 92%, 93%, 94%, 95%, 96%, 97%, 98% or 99% identity to a nucleotide sequence selected from the group consisting of SEQ ID NOs: 9, 11, 13, and 15. In some embodiments, the V_{L} region comprises a nucleotide sequence having at least 95%, 96%, 97%, 98% or 99% identity to a nucleotide sequence selected from the group consisting of SEQ ID NOs: 9, 11, 13, and 15.

The antibody or antigen-binding fragment thereof described herein can comprise antibody constant regions or parts thereof. For example, the heavy chain variable region can be attached at its C-terminal to all or part of a heavy chain like IgA, IgD, IgE, IgG, and IgM, and any isotype subclass. Thus, the antibody or antigen-binding fragment thereof described herein can include the CH1, hinge, CH2, CH3, and/or CH4 regions of the heavy chain.

In some embodiments, of any one of the aspects, the antibody or antigen-binding fragment thereof comprises a heavy chain amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 2, 4 and 6. For example, the antibody or antigen-binding fragment thereof comprises a heavy chain amino acid sequence having at least 90%, 91%, 92%, 93%, 94%, 95%, 96%, 97%, 98% or 99% identity to an amino acid sequence selected from the group consisting of SEQ ID NOs: 2, 4 and 6. In some embodiments, the antibody or antigen-binding fragment thereof comprises a heavy chain amino acid sequence having at least 95%, 96%, 97%, 98% or 99% identity to an amino acid sequence selected from the group consisting of SEQ ID NOs: 2, 4 and 6.

In some embodiments, of any one of the aspects, the antibody or antigen-binding fragment thereof comprises a heavy chain nucleotide sequence having at least 85%, 86%, 87%, 88%, 89%, 90%, 91%, 92%, 93%, 94%, 95%, 96%, 97%, 98% or 99% identity to a nucleotide sequence selected from the group consisting of SEQ ID NOs: 3, 5 and 7. For example, the antibody or antigen-binding fragment thereof comprises a heavy chain nucleotide sequence having at least 90%, 91%, 92%, 93%, 94%, 95%, 96%, 97%, 98% or 99% identity to a nucleotide sequence selected from the group consisting of SEQ ID NOs: 3, 5 and 7. In some embodiments, the antibody or antigen-binding fragment thereof comprises a heavy chain nucleotide sequence having at least 95%, 96%, 97%, 98% or 99% identity to a nucleotide sequence selected from the group consisting of SEQ ID NOs: 3, 5 and 7.

In some embodiments, of any one of the aspects, the antibody or antigen-binding fragment thereof comprises a light chain amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 8, 10, 12 and 14. For example, the antibody or antigen-binding fragment thereof comprises a light chain amino acid sequence having at least 90%, 91%, 92%, 93%, 94%, 95%, 96%, 97%, 98% or 99% identity to an amino acid sequence selected from the group consisting of SEQ ID NOs: 8, 10, 12 and 14. In some embodiments, the antibody or antigen-binding fragment thereof comprises a light chain amino acid sequence having at least 95%, 96%, 97%, 98% or 99% identity to an amino acid sequence selected from the group consisting of SEQ ID NOs: 8, 10, 12 and 14.

In some embodiments, of any one of the aspects, the antibody or antigen-binding fragment thereof comprises a light chain nucleotide sequence having at least 85%, 86%, 87%, 88%, 89%, 90%, 91%, 92%, 93%, 94%, 95%, 96%, 97%, 98% or 99% identity to a nucleotide sequence selected from the group consisting of SEQ ID NOs: 9, 11, 13 and 15. For example, the antibody or antigen-binding fragment thereof comprises a light chain nucleotide sequence having at least 90%, 91%, 92%, 93%, 94%, 95%, 96%, 97%, 98% or 99% identity to a nucleotide sequence selected from the group consisting of SEQ ID NOs: 9, 11, 13 and 15. In some embodiments, the antibody or antigen-binding fragment thereof comprises a light chain nucleotide sequence having at least 95%, 96%, 97%, 98% or 99% identity to a nucleotide sequence selected from the group consisting of SEQ ID NOs: 9, 11, 13 and 15.

In one embodiment, the anti-Notch4 antibody is clone A1472. Clone A1472 comprises a variable heavy chain having an amino acid sequence of SEQ ID NO: 2, or a nucleotide sequence of SEQ ID NO: 3; a variable light chain having an amino acid sequence of SEQ ID NO: 8, or a nucleotide sequence of SEQ ID NO: 9; a CDR_H1 having an amino acid sequence of SEQ ID NO: 16; a CDR_H2 having an amino acid sequence of SEQ ID NO: 17; a CDR_H3 having an amino acid sequence of SEQ ID NO: 18; a CDR_L1 having an amino acid sequence of SEQ ID NO: 19; a CDR_L2 having an amino acid sequence of SEQ ID NO: 20; and a CDR_L3 having an amino acid sequence of SEQ ID NO: 21.

In one embodiment of any aspect herein, the light chain variable region comprises an amino acid sequence having at least 91%, at least 92%, at least 93%, at least 94%, at least 95%, at least 96%, at least 97%, at least 98%, or at least 99% identity identity to an amino acid sequence selected from the group consisting of SEQ ID NOs: 8, 10, 12 or 14.

In one embodiment of any aspect herein, the light chain variable region comprises an amino acid sequence selected from the group consisting of SEQ ID NOs: 8, 10, 12, or 14.

In one embodiment of any aspect herein, the light chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 9, 11, 13, or 15.

In one embodiment of any aspect herein, the light chain variable region comprises an amino acid sequence encoded by a nucleotide sequence selected from the group consisting of SEQ ID NOs: 9, 11, 13, or 15.

In one embodiment of any aspect herein, the antibody comprising
a. a heavy chain variable region (V_{H}) comprising:
   i. a complementarity determining region 1 (CDR_H1) sequence comprising an amino acid sequence that differs by no more than four amino acids from an amino acid sequence selected from the group consisting of SEQ ID NOs 16, 22, 28, and 34;
   ii. a complementarity determining region 2 (CDR_H2) sequence comprising an amino acid sequence that differs by no more than four amino acids from an amino acid sequence selected from the group consisting of SEQ ID NOs 17, 23, 29, and 35;
   iii. a complementarity determining region 3 (CDR_H3) sequence comprising an amino acid sequence that differs by no more than four amino acids from an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence that differs by no more than four amino acids from an amino acid sequence selected from the group consisting of SEQ ID NOs 19, 25, 31, and 37;
   ii. a complementarity determining region 2 (CDR_L2) sequence comprising an amino acid sequence that differs by no more than two amino acids from an amino acid sequence selected from the group consisting of SEQ ID NOs 20, 26, 32, and 38;
   iii. a complementarity determining region 3 (CDR_L3) sequence comprising an amino acid sequence that differs by no more than four amino acids from an amino acid sequence selected from the group consisting of SEQ ID NOs 21, 27, 33, and 39.

In one embodiment of any aspect herein, the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:
a. a heavy chain variable region (V_{H}) comprising:
   i. a complementarity determining region 1 (CDR_H1) sequence comprising an amino acid sequence selected from the group consisting of SEQ ID NOs 16, 22, 28, and 34;
   ii. a complementarity determining region 2 (CDR_H2) sequence comprising an amino acid sequence selected from the group consisting of SEQ ID NOs 17, 23, 29, and 35;
   iii. a complementarity determining region 3 (CDR_H3) sequence comprising an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence selected from the group consisting of SEQ ID NOs 19, 25, 31, and 37;
   ii. a complementarity determining region 2 (CDR_L2) sequence comprising an amino acid sequence selected from the group consisting of SEQ ID NOs 20, 26, 32, and 38;
   iii. a complementarity determining region 3 (CDR_L3) sequence comprising an amino acid sequence selected from the group consisting of SEQ ID NOs 21, 27, 33, and 39.

In one embodiment of any aspect herein, the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:

the antibody or antigen binding fragment thereof comprise:
a. a heavy chain variable region (V_{H}) comprising:
   i. a complementarity determining region 1 (CDR_H1) sequence comprising an amino acid sequence selected from the group consisting of SEQ ID NOs 16, 22, 28, and 34;
   ii. a complementarity determining region 2 (CDR_H2) sequence comprising an amino acid sequence selected from the group consisting of SEQ ID NOs 17, 23, 29, and 35;
   iii. a complementarity determining region 3 (CDR_H3) sequence comprising an amino acid sequence selected from the group consisting of SEQ ID NOs 18, 24, 30, and 36; and
b. a light chain variable region (V_{L}) comprising:
   i. a complementarity determining region 1 (CDR_L1) sequence comprising an amino acid sequence selected from the group consisting of SEQ ID NOs 19, 25, 31, and 37;
   ii. a complementarity determining region 2 (CDR_L2) sequence comprising an amino acid sequence selected from the group consisting of SEQ ID NOs 20, 26, 32, and 38;
   iii. a complementarity determining region 3 (CDR_L3) sequence comprising an amino acid sequence selected from the group consisting of SEQ ID NOs 21, 27, 33, and 39.

In one embodiment, the anti-Notch4 antibody is clone A1527. Clone A1527 comprises a variable light chain having an amino acid sequence of SEQ ID NO: 10, or a nucleotide sequence of SEQ ID NO: 11; a CDR_H1 having an amino acid sequence of SEQ ID NO: 22; a CDR_H2 having an amino acid sequence of SEQ ID NO: 23; a CDR_H3 having an amino acid sequence of SEQ ID NO: 24; a CDR_L1 having an amino acid sequence of SEQ ID NO: 25; a CDR_L2 having an amino acid sequence of SEQ ID NO: 26; and a CDR_L3 having an amino acid sequence of SEQ ID NO: 27.

In one embodiment, the anti-Notch4 antibody is clone A1528. Clone A1528 comprises a variable heavy chain having an amino acid sequence of SEQ ID NO: 4, or a nucleotide sequence of SEQ ID NO: 5; a variable light chain having an amino acid sequence of SEQ ID NO: 12, or a nucleotide sequence of SEQ ID NO: 13; a CDR_H1 having an amino acid sequence of SEQ ID NO: 28; a CDR_H2 having an amino acid sequence of SEQ ID NO: 29; a CDR_H3 having an amino acid sequence of SEQ ID NO: 30; a CDR_L1 having an amino acid sequence of SEQ ID NO: 31; a CDR_L2 having an amino acid sequence of SEQ ID NO: 32; and a CDR_L3 having an amino acid sequence of SEQ ID NO: 33.

In one embodiment, the anti-Notch4 antibody is clone A1529. Clone A1529 comprises a variable heavy chain having an amino acid sequence of SEQ ID NO: 6, or a nucleotide sequence of SEQ ID NO: 7; a variable light chain having an amino acid sequence of SEQ ID NO: 14, or a nucleotide sequence of SEQ ID NO: 15; a CDR_H1 having an amino acid sequence of SEQ ID NO: 34; a CDR_H2 having an amino acid sequence of SEQ ID NO: 35; a CDR_H3 having an amino acid sequence of SEQ ID NO: 36; a CDR_L1 having an amino acid sequence of SEQ ID NO: 37; a CDR_L2 having an amino acid sequence of SEQ ID NO: 38; and a CDR_L3 having an amino acid sequence of SEQ ID NO: 39.

In one embodiment, the amino acid sequence of the full heavy chain sequence of clone A1472 is SEQ ID NO: 40, and the amino acid sequence of the full light chain sequence of clone A1472 is SEQ ID NO: 46. In one embodiment, the nucleotide sequence of the full heavy chain sequence of clone A1472 is SEQ ID NO: 41, and the nucleotide sequence of the full light chain sequence of clone A1472 is SEQ ID NO: 47.

In one embodiment, the amino acid sequence of the full light chain sequence of clone A1527 is SEQ ID NO: 48. In one embodiment, the nucleotide sequence of the full light chain sequence of clone A1527 is SEQ ID NO: 49.

In one embodiment, the amino acid sequence of the full heavy chain sequence of clone A1528 is SEQ ID NO: 42, and the amino acid sequence of the full light chain sequence of clone A1528 is SEQ ID NO: 50. In one embodiment, the nucleotide sequence of the full heavy chain sequence of clone A1528 is SEQ ID NO: 43, and the nucleotide sequence of the full light chain sequence of clone A1528 is SEQ ID NO: 51.

In one embodiment, the amino acid sequence of the full heavy chain sequence of clone A1529 is SEQ ID NO: 44, and the amino acid sequence of the full light chain sequence of clone A1529 is SEQ ID NO: 52. In one embodiment, the nucleotide sequence of the full heavy chain sequence of clone A1529 is SEQ ID NO: 45, and the nucleotide sequence of the full light chain sequence of clone A1529 is SEQ ID NO: 53.

In another embodiment of this aspect and all other aspects provided herein, the antibody or antigen binding fragment reduces Notch4 activity by at least 20% (e.g., at least 30%, at least 40%, at least 50%, at least 60%, at least 70%, at least 80%, at least 90%, at least 95%, at least 98%, at least 99% or even 100% (i.e., complete inhibition or Notch4 activity below detectable limits) as compared to Notch4 in the absence of the antibody or antigen-binding fragment.

In one embodiment, an inhibitory antibody or antigen binding fragment thereof binds the Notch4 (e.g., the Notch4 extracellular domain) and/or inhibits or otherwise interferes with binding of natural ligands to the Notch4 receptor molecule. Other mechanisms, such as interference with receptor interaction with other (co)regulatory molecules can also be effective; the key is that binding of the antibody reagent inhibits receptor signaling, and this can be verified in an appropriate cell culture assay.

A variety of suitable antibody reagent formats are known in the art, such as complete antibodies, e.g., an IgG, or modified forms or fragments of such antibodies, including, as non-limiting examples, single chain antibodies, heterodimers of antibody heavy chains and/or light chains, an Fv fragment (e.g., single chain Fv (scFv), a disulfide bonded Fv), a Fab fragment, a Fab′ fragment, a F(ab′)2 fragment), a single variable domain (e.g., VH, VL, VHH), a dAb, and modified versions of any of the foregoing (e.g., modified by the covalent attachment of polyalkylene glycol (e.g., polyethylene glycol, polypropylene glycol, polybutylene glycol) or other suitable polymer). Antibody reagents or constructs can, if desired, be linked to an antibody Fc region, comprising one or both of CH2 and CH3 domains, and optionally, a hinge region. Such linkage can provide benefits such as increased serum half-life or promotion of effector function(s). Alternatively, antibody reagents or constructs can be fused to a carrier such as serum albumin to promote increased serum half-life.

Antibodies suitable for practicing the methods described herein are preferably monoclonal, and can include, but are not limited to, human, humanized or chimeric antibodies, including single chain antibodies, Fab fragments, F(ab′) fragments, fragments produced by a Fab expression library, and/or binding fragments of any of the above. Antibody reagents also include immunoglobulin molecules and immunologically active portions of immunoglobulin molecules, i.e., molecules that contain at least one, at least two, at least three or more antigen binding sites that specifically bind TIM-3 and one or more myeloid cell markers. Such immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule, as understood by one of skill in the art.

In one embodiment, the antibody that binds Notch4 is a monoclonal antibody.

Additional types of antibodies include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

The term “monoclonal antibody” as used herein refers to a population of antibodies that comprise an identical antigen-binding domain. In some embodiments, a monoclonal antibody can be produced by a single B cell clone, B cell hybrodima or its equivalent. Such a cell produces only one antibody, such that all antibodies produced by such a clone have the same antigen-binding domain. In contrast to polyclonal antibody preparations that typically include different antibodies directed against different determinants (epitopes) on a given target antigen, each monoclonal antibody is directed against a single determinant on the antigen. The term “monoclonal antibody” as used herein is not limited to antibodies produced through hybridoma technology. It is to be understood that the term “monoclonal antibody” refers to an antibody that is derived from a single clone, including any eukaryotic, prokaryotic or phage clone, and not the method by which the antibody is produced. For example, the monoclonal antibodies to be used in accordance with the methods and compositions described herein can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A wide variety of methods for producing constructs with the antigen-binding domain of monoclonal antibodies are known to those of ordinary skill in the art.

Antibodies useful in the present methods can be described or specified in terms of the particular CDRs they comprise (see e.g., Tables 1-3). The compositions and methods described herein encompass the use of an antibody or derivative thereof comprising a heavy or light chain variable domain, where the variable domain comprises a set of three CDRs, and in which the antibody or antibody derivative thereof specifically binds Notch4.

Also provided herein are chimeric antibody derivatives of an anti-Notch4 polypeptide agent, i.e., antibody molecules in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the selected antigens, on the surface of differentiated cells or tumor-specific cells. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

The Notch4 antibodies described herein can also include humanized antibody derivatives. Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, antibodies described herein include derivatives that are modified, i.e., by the covalent attachment of another type of molecule to the antibody that does not prevent the antibody from binding to its target. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

Antibodies or antigen-binding fragments thereof that bind Notch4 as described herein can be generated by any suitable method known in the art. Antibodies can be produced in bacteria, yeast, fungi, protozoa, insect cells, plants, or mammalian cells (see e.g., Frenzel et al. (2013) Front Immunol. 4: 217). A mammalian expression system is generally preferred for manufacturing most of therapeutic proteins, such as antibodies, as they require post-translational modifications. A variety of mammalian cell expression systems are now available for expression of antibodies, including but not limited to immortalized Chinese hamster ovary (CHO) cells, mouse myeloma (NSO), mouse L-cells, myeloma cell lines like J558L and Sp2/0, baby hamster kidney (BHK), or human embryo kidney (HEK-293).

In certain embodiments, the Notch4 antibodies described herein are produced in antibody producing cells isolated and/or derived from a human or mammalian subject, which permits manipulation and serves as the basis for production of mammalian, including human, hybridoma cell lines. Typically, the host animal is inoculated intraperitoneally with an amount of immunogen, e.g., Raji cells or SCR1/SCR2 fusion protein, sufficient to generate an immunogenic response and then boosted with similar amounts of the immunogen. Lymphoid cells, preferably spleen lymphoid cells from the host, are collected a few days after the final boost and a cell suspension is prepared therefrom for use in the fusion.

In some embodiments, the Notch4 antibody or antigen-binding fragment thereof is a humanized antibody. Methods for generating humanized antibodies are known to those of skill in the art and are not described in detail herein. The following four general steps for generating a humanized antibody include: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; and 6,331,415.

In yet another alternative, fully human antibodies can be obtained by using commercially available mice that have been engineered to express specific human immunoglobulin proteins. Transgenic animals that are designed to produce a more desirable (e.g., fully human antibodies) or more robust immune response can also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N. J.).

In an alternative, antibodies can be made recombinantly and expressed using any method known in the art. Antibodies can be made recombinantly by expressing the gene sequence of a given anti-Notch4 antibody recombinantly in host cells (e.g., CHO cells). Another method which can be employed is to express the antibody sequence in plants (e.g., tobacco) or transgenic milk. Methods for expressing antibodies recombinantly in plants or milk have been disclosed. See, for example, Peeters, et al. (2001) Vaccine 19:2756; Lonberg, N. and D. Huszar (1995) Int. Rev. Immunol 13:65; and Pollock, et al. (1999) J Immunol Methods 231:147. Methods for making derivatives of antibodies, e.g., humanized, single chain, etc. are known in the art. In another alternative, antibodies can be made recombinantly by phage display technology. See, for example, U.S. Pat. Nos. 5,565,332; 5,580,717; 5,733,743; 6,265,150; and Winter et al., Annu. Rev. Immunol. (1994) 12:433-455. In addition, binding of Notch4 binding proteins to cells expressing Notch4 can be characterized in a number assays known in the art, including FACS (Fluorescence Activated Cell Sorting), immunofluorescence, and immunocytochemistry. Notch4 binding protein is contacted with cells which express or contain Notch4 (e.g., B cells), and binding is detected in accordance with the method being used.

In certain embodiments, the anti-Notch4 antibodies (or antigen binding fragment thereof) described herein exhibits 1, 2, 3, or 4 of the following characteristics: (a) binds to Notch4; (b) binds to one or more epitopes of Notch4 to which a ligand binds; (c) binds to Notch4 to inhibit Notch activation (d) binds to Notch4 to inhibit Notch4 activation.

Immunoassays and flow cytometry sorting techniques such as fluorescence activated cell sorting (FACS) can also be employed to isolate antibodies that are specific for Notch4. For example, ELISA with Notch ligand-coated and soluble Notch4-coated plates can be employed to determine which antibodies are specific for ligand binding portion of Notch4. Flow cytometry can be used to assess how well the antibody(-ies) bind to Notch4-expressing cells, including but not limited to B cells or a cell line such as Raji. In the alternative, antibodies can be screened by combining with a population of B cells and then exposing the B cells to a source of a Notch4 ligand, either in isolated form (e.g. ligand-coated plates) or in natural form (e.g., in serum).

As used herein, the term “Complementarity Determining Regions” (CDRs, i.e., CDR1, CDR2, and CDR3) refers to the amino acid residues of an antibody variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain). Likewise, “frameworks” (FWs) comprise amino acids 1-23 (FW1), 35-49 (FW2), 57-88 (FW3), and 98-107 (FW4) in the light chain variable domain and 1-30 (FW1), 36-49 (FW2), 66-94 (FW3), and 103-113 (FW4) in the heavy chain variable domain taking into account the Kabat numbering system (Kabat et al., Sequences of Proteins of Immunological Interest, 5th Ed. Public Health Service, National Institutes of Health, Bethesda, Md. (1987, 1991)).

The Kabat residue designations do not always correspond directly with the linear numbering of the amino acid residues. The actual linear amino acid sequence can contain fewer or additional amino acids than in the strict Kabat numbering corresponding to a shortening of, or insertion into, a structural component, whether framework or complementarity determining region (CDR), of the basic variable domain structure. The correct Kabat numbering of residues can be determined for a given antibody by alignment of residues of homology in the sequence of the antibody with a “standard” Kabat numbered sequence. Methods and computer programs for determining sequence similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm can also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

TABLE 1
Exemplary variable heavy chain sequences of Notch4
antibodies as described herein.

| | | SEQ ID |
|---|---|---|
| Clone | Variable Heavy Chain Sequence | NO: |
| A1472 | QVQLQQPGAELVKPGASVKLSCKASGYTFINYWMLWVKQRPGQGLEWIGMIHPNS | 2 |
| | VSTNYNEKFKSKATLTVDKSSSTAYMQLSSLTSEDSAVYYCGTGRDYFDYWGQGT | |
| | TLTVSS | |
| A1472 | CAGGTCCAACTTCAACAGCCTGGGGCTGAGCTGGTAAAGCCTGGGGCTTCAGTGA | 3 |
| | AGTTGTCCTGCAAGGCTTCTGGCTACACTTTTATCAATTACTGGATGCTCTGGGT | |
| | GAAGCAGAGGCCTGGACAAGGCCTTGAGTGGATTGGTATGATTCATCCTAATAGT | |
| | GTTAGTACTAACTACAATGAGAAGTTCAAGAGCAAGGCCACACTGACTGTAGACA | |
| | AATCCTCCAGCACAGCCTACATGCAACTCAGCAGCCTGACATCTGAGGACTCTGC | |
| | GGTCTATTACTGTGGAACTGGGAGGGACTACTTTGACTACTGGGGCCAAGGCACC | |
| | ACTCTCACAGTCTCCTCA | |
| A1528 | QVTLKESGPGILQPSQTLNLTCSFSGFSLSTFGMGVGWIRQPSGKGLEWLAHIWW | 4 |
| | DDEMYYNPVLKSRLTISKETSKTQVFLKIATVDTADTATYYCVRRRRGLAMDSWG | |
| | HGTSVTVSS | |
| A1528 | CAGGTTACTCTGAAAGAGTCTGGCCCTGGGATATTGCAGCCCTCCCAGACCCTCA | 5 |
| | ATCTGACTTGTTCTTTCTCTGGGTTTTCACTGAGCACTTTTGGTATGGGTGTGGG | |
| | CTGGATTCGTCAGCCTTCAGGGAAGGGTCTGGAGTGGCTGGCACACATTTGGTGG | |
| | GATGATGAAATGTACTATAACCCAGTCCTGAAGAGTCGGCTCACAATCTCCAAGG | |
| | AAACCTCCAAAACCCAGGTATTCCTCAAGATCGCCACTGTGGACACTGCAGATAC | |
| | TGCCACATATTACTGTGTTCGAAGACGACGCGGGTTGGCTATGGACTCCTGGGGT | |
| | CACGGAACCTCAGTCACCGTCTCCTCA | |
| A1529 | DVQLQESGPGLVKPSQSLSLTCSVTGYSITRGFYWNWIRQFPGNKLEWMGYISYD | 6 |
| | GSNNYNPSLKNRISITRDTSKNQFFLQLNSVTTEDTATYYCARMDYWGQGTSVTV | |
| | SS | |
| A1529 | GATGTACAGCTTCAGGAGTCAGGACCTGGCCTCGTGAAACCTTCTCAGTCTCTGT | 7 |
| | CTCTCACCTGCTCTGTCACTGGCTACTCCATCACCAGAGGTTTTTACTGGAACTG | |
| | GATCCGGCAGTTTCCAGGAAACAAACTGGAATGGATGGGCTACATAAGCTACGAC | |
| | GGTAGCAATAACTACAACCCATCTCTCAAAAATCGAATCTCCATCACTCGTGACA | |
| | CATCTAAGAACCAGTTTTTCCTGCAGTTGAATTCTGTGACTACTGAGGACACAGC | |
| | TACATATTACTGTGCAAGGATGGACTACTGGGGTCAAGGAACCTCAGTCACCGTC | |
| | TCCTCA | |

TABLE 2
Exemplary variable light chain sequences of Notch4
antibodies as described herein.

| | | SEQ ID |
|---|---|---|
| Clone | Variable Light Chain Sequence | NO: |
| A1472 | DIVMTQSHKFMSTSVGDRVSITCKASQDVGTAVAWYQQKPGQSPKLLIYWASTRH | 8 |
| | TGVPDRFTGSGSGTDFTLTIGNVQSEDLADYFCQQYSSYPFTFGSGTKLEIK | |
| A1472 | GACATTGTGATGACCCAGTCTCACAAATTCATGTCCACATCAGTAGGAGACAGGG | 9 |
| | TCAGCATCACCTGCAAGGCCAGTCAGGATGTGGGTACTGCTGTAGCCTGGTATCA | |
| | ACAGAAACCAGGGCAATCTCCTAAACTACTGATTTACTGGGCATCCACCCGGCAC | |
| | ACTGGAGTCCCTGATCGCTTCACAGGCAGTGGATCTGGGACAGATTTCACTCTCA | |
| | CCATTGGCAATGTGCAGTCTGAAGACTTGGCAGATTATTTCTGTCAGCAATATAG | |
| | CAGCTATCCATTCACGTTCGGCTCGGGGACAAAGTTGGAAATAAAA | |
| A1527 | DIQMTOTTSSLSASLGDRVTISCRASQDISNYLNWYQQKPDGTVKLLIYYTSRLH | 10 |
| | SGVPSRFSGSGSGTDYSLTISNLEQEDIATYFCQQGNTLPWTFGGGTKLEIK | |
| A1527 | GATATCCAGATGACACAGACTACATCCTCCCTGTCTGCCTCTCTGGGAGACAGAG | 11 |
| | TCACCATCAGTTGCAGGGCAAGTCAGGACATTAGCAATTATTTAAACTGGTATCA | |
| | GCAGAAACCAGATGGAACTGTTAAACTCCTGATCTACTACACATCAAGATTACAC | |
| | TCAGGAGTCCCATCAAGGTTCAGTGGCAGTGGGTCTGGAACAGATTATTCTCTCA | |
| | CCATTAGCAACCTGGAGCAAGAAGATATTGCCACTTACTTTTGCCAACAGGGTAA | |
| | TACGCTTCCGTGGACGTTCGGTGGAGGCACCAAGCTGGAAATCAAA | |
| A1528 | DIVMTQSHKFMSTSIGDRVNITCKASQDVTTAVAWYQQKPGQSPKLLIYSASYRY | 12 |
| | TGIPDRFTGTGSGTDFTFTISTVQAEDLALYYCQQHSNTPLTFGAGTKLELK | |
| A1528 | GACATTGTGATGACCCAGTCTCACAAATTCATGTCCACATCAATAGGAGACAGGG | 13 |
| | TCAACATCACCTGCAAGGCCAGTCAGGATGTGACTACTGCTGTAGCCTGGTATCA | |
| | ACAGAAACCAGGACAATCTCCTAAACTACTGATTTACTCGGCATCCTACCGATAC | |
| | ACTGGAATCCCTGATCGCTTCACTGGCACTGGATCTGGGACGGATTTCACTTTCA | |
| | CCATCAGCACTGTGCAGGCTGAAGACCTGGCACTTTATTACTGTCAGCAACATTC | |
| | TAATACTCCGCTCACGTTCGGTGCTGGGACCAAGCTGGAGCTGAAA | |
| A1529 | EIVLTQSPALMAASPGEKVTITCSVSSSISSSNLHWYQQKSETSPKPWIYGTSNL | 14 |
| | ASGVPVRFSGSGSGTSYSLTISSMEAEDAATYYCQQWSSYPFTFGSGTKLEIK | |
| A1529 | GAAATTGTGCTCACCCAGTCTCCAGCACTCATGGCTGCATCTCCAGGGGAGAAGG | 15 |
| | TCACCATCACCTGCAGTGTCAGCTCAAGTATAAGTTCCAGCAACTTGCACTGGTA | |
| | CCAGCAGAAGTCAGAAACCTCCCCCAAACCCTGGATTTATGGCACATCCAACCTG | |
| | GCTTCTGGAGTCCCTGTTCGCTTCAGTGGCAGTGGATCTGGGACCTCTTATTCTC | |
| | TCACAATCAGCAGCATGGAGGCTGAAGATGCTGCCACTTATTACTGTCAACAGTG | |
| | GAGTAGTTACCCATTCACGTTCGGCTCGGGGACAAAGTTGGAAATAAAA | |

TABLE 3
Exemplary CDR (heavy and light) sequences of
Notch4 antibodies as described herein.

| Clone | CDR | Sequence | SEQ ID NO: |
|---|---|---|---|
| A1472 | CDR_H1 | GYTFINYW.... | 16 |
| A1472 | CDR_H2 | IHPNSVST.. | 17 |
| A1472 | CDR_H3 | GTGRDYFDY | 18 |
| A1472 | CDR_L1 | QDVGTA...... | 19 |
| A1472 | CDR_L2 | WAS....... | 20 |
| A1472 | CDR_L3 | QQYSSYPFT | 21 |
| A1527 | CDR_H1 | AYTFSDHS.... | 22 |
| A1527 | CDR_H2 | INTETGEP.. | 23 |
| A1527 | CDR_H3 | TRGGHYFFDY | 24 |
| A1527 | CDR_L1 | QDISNY...... | 25 |
| A1527 | CDR_L2 | YTS...... | 26 |
| A1527 | CDR_L3 | QQGNTLPWT | 27 |
| A1528 | CDR_H1 | GFSLSTFGMG.... | 28 |
| A1528 | CDR_H2 | IWWDDEM.. | 29 |
| A1528 | CDR_H3 | VRRRRGLAMDS | 30 |
| A1528 | CDR_L1 | QDVTTA...... | 31 |
| A1528 | CDR_L2 | SAS....... | 32 |
| A1528 | CDR_L3 | QQHSNTPLT | 33 |
| A1529 | CDR_H1 | GYSITRGFY.... | 34 |
| A1529 | CDR_H2 | ISYDGSN.. | 35 |
| A1529 | CDR_H3 | ARMDY | 36 |
| A1529 | CDR_L1 | SSISSSN.... | 37 |
| A1529 | CDR_L2 | GTS.. | 38 |
| A1529 | CDR_L3 | QQWSSYPFT | 39 |

TABLE 4
Exemplary full heavy chain sequences of Notch4
antibodies as described herein.

| | | SEQ ID |
|---|---|---|
| Clone | Full Heavy Chain Sequence | NO: |
| A1472 | MRSQFSLQLLSTQDLTVGWSYIILFLVATATGVHSQVQLQQPGAELVKPGASVKL | 40 |
| | SCKASGYTFINYWMLWVKQRPGQGLEWIGMIHPNSVSTNYNEKFKSKATLTVDKS | |
| | SSTAYMQLSSLTSEDSAVYYCGTGRDYFDYWGQGTTLTVSSAKTTPPSVYPLAPG | |
| | SAAQTNSMVTLGCLVKGYFPEPVTVTWNSGSLSSGVHTFPAVLQSDLYTLSSSVT | |
| | VPSS | |
| A1472 | ATGAGATCACAGTTCTCTCTACAGTTACTGAGCACACAGGACCTCACAGTGGGAT | 41 |
| | GGAGCTATATCATCCTCTTTTTGGTAGCAACAGCTACAGGTGTCCACTCCCAGGT | |
| | CCAACTTCAACAGCCTGGGGCTGAGCTGGTAAAGCCTGGGGCTTCAGTGAAGTTG | |
| | TCCTGCAAGGCTTCTGGCTACACTTTTATCAATTACTGGATGCTCTGGGTGAAGC | |
| | AGAGGCCTGGACAAGGCCTTGAGTGGATTGGTATGATTCATCCTAATAGTGTTAG | |
| | TACTAACTACAATGAGAAGTTCAAGAGCAAGGCCACACTGACTGTAGACAAATCC | |
| | TCCAGCACAGCCTACATGCAACTCAGCAGCCTGACATCTGAGGACTCTGCGGTCT | |
| | ATTACTGTGGAACTGGGAGGGACTACTTTGACTACTGGGGCCAAGGCACCACTCT | |
| | CACAGTCTCCTCAGCCAAAACGACACCCCCATCTGTCTATCCACTGGCCCCTGGA | |
| | TCTGCTGCCCAAACTAACTCCATGGTGACCCTGGGATGCCTGGTCAAGGGCTATT | |
| | TCCCTGAGCCAGTGACAGTGACCTGGAACTCTGGATCCCTGTCCAGCGGTGTGCA | |
| | CACCTTCCCAGCTGTCCTGCAGTCTGACCTCTACACTCTGAGCAGCTCAGTGACT | |
| | GTCCCCTCCAGC | |
| A1528 | MGRLTSSFLLLTVPAYVLSQVTLKESGPGILQPSQTLNLTCSFSGFSLSTFGMGV | 42 |
| | GWIRQPSGKGLEWLAHIWWDDEMYYNPVLKSRLTISKETSKTQVFLKIATVDTAD | |
| | TATYYCVRRRRGLAMDSWGHGTSVTVSSAKTTPPSVYPLAPGSAAQTNSMVTLGC | |
| | LVKGYFPEPVTVTWNSGSLSSGVHTFPAVLQSDLYTLSSSVTVPSS | |
| A1528 | ATGGGCAGGCTTACTTCTTCATTCCTGTTACTGACTGTCCCTGCATATGTCCTGT | 43 |
| | CCCAGGTTACTCTGAAAGAGTCTGGCCCTGGGATATTGCAGCCCTCCCAGACCCT | |
| | CAATCTGACTTGTTCTTTCTCTGGGTTTTCACTGAGCACTTTTGGTATGGGTGTG | |
| | GGCTGGATTCGTCAGCCTTCAGGGAAGGGTCTGGAGTGGCTGGCACACATTTGGT | |
| | GGGATGATGAAATGTACTATAACCCAGTCCTGAAGAGTCGGCTCACAATCTCCAA | |
| | GGAAACCTCCAAAACCCAGGTATTCCTCAAGATCGCCACTGTGGACACTGCAGAT | |
| | ACTGCCACATATTACTGTGTTCGAAGACGACGCGGGTTGGCTATGGACTCCTGGG | |
| | GTCACGGAACCTCAGTCACCGTCTCCTCAGCCAAAACGACACCCCCATCTGTCTA | |
| | TCCACTGGCCCCTGGATCTGCTGCCCAAACTAACTCCATGGTGACCCTGGGATGC | |
| | CTGGTCAAGGGCTATTTCCCTGAGCCAGTGACAGTGACCTGGAACTCTGGATCCC | |
| | TGTCCAGCGGTGTGCACACCTTCCCAGCTGTCCTGCAGTCTGACCTCTACACTCT | |
| | GAGCAGCTCAGTGACTGTCCCCTCCAGCTG | |
| A1529 | MKVLSLLYLLTAIPGILSDVQLQESGPGLVKPSQSLSLTCSVTGYSITRGFYWNW | 44 |
| | IRQFPGNKLEWMGYISYDGSNNYNPSLKNRISITRDTSKNQFFLQLNSVTTEDTA | |
| | TYYCARMDYWGQGTSVTVSSAKTTPPSVYPLAPGSAAQTNSMVTLGCLVKGYFPE | |
| | PVTVTWNSGSLSSGVHTFPAVLQSDLYTLSSSVTVPSS | |
| A1529 | ATGAAAGTGTTGAGTCTGTTGTACCTGTTGACAGCCATTCCTGGTATCCTGTCTG | 45 |
| | ATGTACAGCTTCAGGAGTCAGGACCTGGCCTCGTGAAACCTTCTCAGTCTCTGTC | |
| | TCTCACCTGCTCTGTCACTGGCTACTCCATCACCAGAGGTTTTTACTGGAACTGG | |
| | ATCCGGCAGTTTCCAGGAAACAAACTGGAATGGATGGGCTACATAAGCTACGACG | |
| | GTAGCAATAACTACAACCCATCTCTCAAAAATCGAATCTCCATCACTCGTGACAC | |
| | ATCTAAGAACCAGTTTTTCCTGCAGTTGAATTCTGTGACTACTGAGGACACAGCT | |
| | ACATATTACTGTGCAAGGATGGACTACTGGGGTCAAGGAACCTCAGTCACCGTCT | |
| | CCTCAGCCAAAACGACACCCCCATCTGTCTATCCACTGGCCCCTGGATCTGCTGC | |
| | CCAAACTAACTCCATGGTGACCCTGGGATGCCTGGTCAAGGGCTATTTCCCTGAG | |
| | CCAGTGACAGTGACCTGGAACTCTGGATCCCTGTCCAGCGGTGTGCACACCTTCC | |
| | CAGCTGTCCTGCAGTCTGACCTCTACACTCTGAGCAGCTCAGTGACTGTCCCCTC | |
| | CAGCTG | |

TABLE 5
Exemplary full light chain sequences of Notch4
antibodies as described herein.

| | | SEQ ID |
|---|---|---|
| Clone | Full Light Chain Sequence | NO: |
| A1472 | METHSQVFVYTLLWLSGVEGDIVMTQSHKFMSTSVGDRVSITCKASQDVGTAVAW | 46 |
| | YQQKPGQSPKLLIYWASTRHTGVPDRFTGSGSGTDFTLTIGNVQSEDLADYFCQQ | |
| | YSSYPFTFGSGTKLEIKRADAAPTVSIFPPSSEQLTSGGASVVCFLNNFYPKDIN | |
| | VKWKIDGSERQNGVLNSWTDQDSKDSTYSMSSTLTLTK | |
| A1472 | ATGGAGACACATTCTCAGGTCTTTGTATACACGTTGCTGTGGTTGTCTGGTGTTG | 47 |
| | AAGGAGACATTGTGATGACCCAGTCTCACAAATTCATGTCCACATCAGTAGGAGA | |
| | CAGGGTCAGCATCACCTGCAAGGCCAGTCAGGATGTGGGTACTGCTGTAGCCTGG | |
| | TATCAACAGAAACCAGGGCAATCTCCTAAACTACTGATTTACTGGGCATCCACCC | |
| | GGCACACTGGAGTCCCTGATCGCTTCACAGGCAGTGGATCTGGGACAGATTTCAC | |
| | TCTCACCATTGGCAATGTGCAGTCTGAAGACTTGGCAGATTATTTCTGTCAGCAA | |
| | TATAGCAGCTATCCATTCACGTTCGGCTCGGGGACAAAGTTGGAAATAAAACGGG | |
| | CTGATGCTGCACCAACTGTATCCATCTTCCCACCATCCAGTGAGCAGTTAACATC | |
| | TGGAGGTGCCTCAGTCGTGTGCTTCTTGAACAACTTCTACCCCAAAGACATCAAT | |
| | GTCAAGTGGAAGATTGATGGCAGTGAACGACAAAATGGCGTCCTGAACAGTTGGA | |
| | CTGATCAGGACAGCAAAGACAGCACCTACAGCATGAGCAGCACCCTCACGTTGAC | |
| | CAAG | |
| A1527 | MSSAQFLGLLLLCFQGTRCDIQMTQTTSSLSASLGDRVTISCRASQDISNYLNWY | 48 |
| | QQKPDGTVKLLIYYTSRLHSGVPSRFSGSGSGTDYSLTISNLEQEDIATYFCQQG | |
| | NTLPWTFGGGTKLEIKRADAAPTVSIFPPSSEQLTSGGASVVCFLNNFYPKDINV | |
| | KWKIDGSERQNGVLNSWTDQDSKDSTYSMSSTLTLTK | |
| A1527 | ATGTCCTCTGCTCAGTTCCTTGGTCTCCTGTTGCTCTGTTTTCAAGGTACCAGAT | 49 |
| | GTGATATCCAGATGACACAGACTACATCCTCCCTGTCTGCCTCTCTGGGAGACAG | |
| | AGTCACCATCAGTTGCAGGGCAAGTCAGGACATTAGCAATTATTTAAACTGGTAT | |
| | CAGCAGAAACCAGATGGAACTGTTAAACTCCTGATCTACTACACATCAAGATTAC | |
| | ACTCAGGAGTCCCATCAAGGTTCAGTGGCAGTGGGTCTGGAACAGATTATTCTCT | |
| | CACCATTAGCAACCTGGAGCAAGAAGATATTGCCACTTACTTTTGCCAACAGGGT | |
| | AATACGCTTCCGTGGACGTTCGGTGGAGGCACCAAGCTGGAAATCAAACGGGCTG | |
| | ATGCTGCACCAACTGTATCCATCTTCCCACCATCCAGTGAGCAGTTAACATCTGG | |
| | AGGTGCCTCAGTCGTGTGCTTCTTGAACAACTTCTACCCCAAAGACATCAATGTC | |
| | AAGTGGAAGATTGATGGCAGTGAACGACAAAATGGCGTCCTGAACAGTTGGACTG | |
| | ATCAGGACAGCAAAGACAGCACCTACAGCATGAGCAGCACCCTCACGTTGACCAA | |
| | G | |
| A1528 | MESQIQVFVFVFLWLSGVDGDIVMTQSHKFMSTSIGDRVNITCKASQDVTTAVAW | 50 |
| | YQQKPGQSPKLLIYSASYRYTGIPDRFTGTGSGTDFTFTISTVQAEDLALYYCQQ | |
| | HSNTPLTFGAGTKLELKRADAAPTVSIFPPSSEQLTSGGASVVCFLNNFYPKDIN | |
| | VKWKIDGSERQNGVLNSWTDQDSKDSTYSMSSTLTLTK | |
| A1528 | ATGGAGTCACAGATTCAGGTCTTTGTATTCGTGTTTCTCTGGTTGTCTGGTGTTG | 51 |
| | ACGGAGACATTGTGATGACCCAGTCTCACAAATTCATGTCCACATCAATAGGAGA | |
| | CAGGGTCAACATCACCTGCAAGGCCAGTCAGGATGTGACTACTGCTGTAGCCTGG | |
| | TATCAACAGAAACCAGGACAATCTCCTAAACTACTGATTTACTCGGCATCCTACC | |
| | GATACACTGGAATCCCTGATCGCTTCACTGGCACTGGATCTGGGACGGATTTCAC | |
| | TTTCACCATCAGCACTGTGCAGGCTGAAGACCTGGCACTTTATTACTGTCAGCAA | |
| | CATTCTAATACTCCGCTCACGTTCGGTGCTGGGACCAAGCTGGAGCTGAAACGGG | |
| | CTGATGCTGCACCAACTGTATCCATCTTCCCACCATCCAGTGAGCAGTTAACATC | |
| | TGGAGGTGCCTCAGTCGTGTGCTTCTTGAACAACTTCTACCCCAAAGACATCAAT | |
| | GTCAAGTGGAAGATTGATGGCAGTGAACGACAAAATGGCGTCCTGAACAGTTGGA | |
| | CTGATCAGGACAGCAAAGACAGCACCTACAGCATGAGCAGCACCCTCACGTTGAC | |
| | CAAG | |
| A1529 | MDFHVQIFSFMLISVTVILSSGEIVLTQSPALMAASPGEKVTITCSVSSSISSSN | 52 |
| | LHWYQQKSETSPKPWIYGTSNLASGVPVRFSGSGSGTSYSLTISSMEAEDAATYY | |
| | CQQWSSYPFTFGSGTKLEIKRADAAPTVSIFPPSSEQLTSGGASVVCFLNNFYPK | |
| | DINVKWKIDGSERQNGVLNSWTDQDSKDSTYSMSSTLTLTK | |
| A1529 | ATGGATTTTCATGTGCAGATTTTCAGCTTCATGCTAATCAGTGTCACAGTCATAT | 53 |
| | TGTCCAGTGGAGAAATTGTGCTCACCCAGTCTCCAGCACTCATGGCTGCATCTCC | |
| | AGGGGAGAAGGTCACCATCACCTGCAGTGTCAGCTCAAGTATAAGTTCCAGCAAC | |
| | TTGCACTGGTACCAGCAGAAGTCAGAAACCTCCCCCAAACCCTGGATTTATGGCA | |
| | CATCCAACCTGGCTTCTGGAGTCCCTGTTCGCTTCAGTGGCAGTGGATCTGGGAC | |
| | CTCTTATTCTCTCACAATCAGCAGCATGGAGGCTGAAGATGCTGCCACTTATTAC | |
| | TGTCAACAGTGGAGTAGTTACCCATTCACGTTCGGCTCGGGGACAAAGTTGGAAA | |
| | TAAAACGGGCTGATGCTGCACCAACTGTATCCATCTTCCCACCATCCAGTGAGCA | |
| | GTTAACATCTGGAGGTGCCTCAGTCGTGTGCTTCTTGAACAACTTCTACCCCAAA | |
| | GACATCAATGTCAAGTGGAAGATTGATGGCAGTGAACGACAAAATGGCGTCCTGA | |
| | ACAGTTGGACTGATCAGGACAGCAAAGACAGCACCTACAGCATGAGCAGCACCCT | |
| | CACGTTGACCAAG | |

Immunoglobin Notch4 binding proteins (e.g., IgG or Fab Notch4 binding proteins) can be modified to reduce immunogenicity. Reduced immunogenicity is desirable in Notch4 binding proteins intended for use as therapeutics, as it reduces the chance that the subject will develop an immune response against the therapeutic molecule. Techniques useful for reducing immunogenicity of Notch4 binding proteins include deletion/modification of potential human T cell epitopes and ‘germlining’ of sequences outside of the CDRs (e.g., framework and Fc). Such methods are known to those of skill in the art and are not described in detail herein.

Standard recombinant nucleic acid methods can be used to express a protein that binds to Notch4. Generally, a nucleic acid sequence encoding the protein is cloned into a nucleic acid expression vector. Of course, if the protein includes multiple polypeptide chains, each chain can be cloned into an expression vector, e.g., the same or different vectors, that are expressed in the same or different cells.

In certain embodiments, the antibodies described herein are bound to one or more carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies can also be conjugated to a detectable agent. The complex is useful to detect the antigens to which the antibody specifically binds in a sample, using standard immunochemical techniques such as flow cytometry or immunohistochemistry as described by Harlow and Lane (1988) supra. Detectable markers can also be used to ascertain binding specificity for a type of cell (e.g., T cell) by using the detectable marker with another marker which is definitive for T cells and analyzing the staining patterns by FACS. There are many different labels and methods of labeling known to those of ordinary skill in the art. Examples of the types of labels which can be used with the antibodies described herein include radioisotopes, enzymes, colloidal metals, fluorescent compounds (e.g., FITC, PE, PECy5, APC, etc.), bioluminescent compounds, and chemiluminescent compounds. Those of ordinary skill in the art will know of other suitable labels for binding to the antibody, or will be able to ascertain such, using routine experimentation. Furthermore, the binding of these labels to an antibody as described herein can be done using standard techniques common to those of ordinary skill in the art.

### Polynucleotides

The disclosure also provides a polynucleotide encoding an antibody or antigen-binding fragment thereof described herein. The skilled person will understand that, due to the degeneracy of the genetic code, a given antibody or antigen-binding fragment thereof can be encoded by different polynucleotides. These “variants” are encompassed herein.

In some embodiments, a nucleic acid encoding an antibody or antigen-binding fragment thereof described herein is comprised in a vector. In some embodiments, a nucleic acid sequence encoding an antibody or antigen-binding fragment thereof described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding an antibody or antigen-binding fragment thereof as described herein in place of non-essential viral genes. The vector and/or particle may be utilized for the purpose of transferring any nucleic acids into cells either in vitro or in vivo. Numerous forms of viral vectors are known in the art.

### Cells

The disclosure also provides a cell comprising an antibody or an antigen binding fragment described herein. The disclosure further provides a host cell comprising a polynucleotide described herein or a plasmid or vector described herein. As used herein, the term “cell” refers to a single cell as well as to a population of (i.e., more than one) cells.

A host cell can be a prokaryotic or eukaryotic host cell. Exemplary host cells include, but are not limited to, bacterial cells, yeast cells, plant cell, animal (including insect) or human cells. The host cells can be employed in a method of producing an antibody or antigen-binding fragment thereof described herein. Generally, the method comprises: culturing a host cell comprising a polynucleotide described herein or a plasmid or vector described herein under conditions such that the antibody or antigen-binding fragment thereof is expressed; and optionally recovering the antibody or antigen-binding fragment thereof from the culture medium. The antibody or antigen-binding fragment thereof can be concentrated and purified by a variety of biochemical and chromatographic methods, including methods utilizing differences in size, charge, hydrophobicity, solubility, specific affinity, etc. between the antibody or antigen-binding fragment thereof and other substances in the cell culture medium. In some embodiments, the antibody or antigen-binding fragment thereof is secreted from the host cells.

The antibody or antigen-binding fragment thereof described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, N Y (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

### Administering to a Cell

The antibody of antigen binding fragment described herein can inhibit or reduce Notch4 activity in a cell, e.g., a Treg cell. Accordingly, an antibody or antigen binding fragment described herein, or a polynucleotide encoding the same, can be administered to a cell, e.g., a Treg cell for inhibiting or reducing Notch4 levels, activity, signaling, etc.

It is noted that administering to the cell can be in vitro or in-vivo. Methods for administering antibodies, antigen binding fragments and polynucleotides to a cell are well known and available to one of skill in the art. As used herein, administering to the cell means contacting the cell with the antibody or antigen binding fragment described herein, or a polynucleotide encoding the same, so that the antibody, the antigen binding fragment or the polynucleotide is taken up by the cell. Generally, the cell can be contacted with the antibody, the antigen binding fragment or the polynucleotide in a cell culture e.g., in vitro or ex vivo, or the antibody, the antigen binding fragment or the polynucleotide can be administrated to a subject, e.g., in vivo. The term “contacting” or “contact” as used herein in connection with contacting a cell includes subjecting the cells to an appropriate culture media, which comprises the antibody, the antigen binding fragment or the polynucleotide. Where the cell is in vivo, “contacting” or “contact” includes administering the compound, e.g., in a pharmaceutical composition to a subject via an appropriate administration route such that the compound contacts the cell in vivo.

For example, when the cell is in vitro, said administering to the cell can include subjecting the cell to an appropriate culture media which comprises the antibody, the antigen binding fragment or the polynucleotide. Where the cell is in vivo, said administering to the cell includes administering the antibody, the antigen binding fragment or the polynucleotide to a subject via an appropriate administration route such that the antibody, the antigen binding fragment or the polynucleotide is administered to the cell in vivo.

### Compositions, Formulations and Packaging

Also provided herein are compositions, including pharmaceutical compositions, comprising an antibody or antigen-binding fragment that binds Notch4 as described herein or a polynucleotide encoding the antibody or antigen-binding fragment. In one embodiment, the compositions are pharmaceutical compositions. Pharmaceutical compositions for use with the methods described herein can be formulated in a conventional manner using one or more physiologically acceptable carriers or excipients. Thus, the compounds and their physiologically acceptable salts and solvates can be formulated for administration by, for example, by aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

Techniques and formulations generally can be found in Remington's Pharmaceutical Sciences, Meade Publishing Co., Easton, PA. For systemic administration, injection is preferred, including intramuscular, intravenous, intraperitoneal, and subcutaneous. For injection, the anti-Notch4 antibodies described herein can be formulated in liquid solutions, preferably in physiologically compatible buffers such as Hank's solution or Ringer's solution. In addition, the antibodies can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Aerosol Formulations

A composition comprising an agent that inhibits Notch4 can be administered directly to the airways of a subject in the form of an aerosol or by nebulization. For use as aerosols, an agent that inhibits Notch4 in solution or suspension may be packaged in a pressurized aerosol container together with suitable propellants, for example, hydrocarbon propellants like propane, butane, or isobutane with conventional adjuvants. An agent that inhibits Notch4 can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits Notch4. Exemplary gases including, but are not limited to, nitrogen, argon or helium can be used to high advantage.

In some embodiments, an agent that inhibits Notch4 can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an agent that inhibits Notch4 thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The anti-Notch4 antibodies can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the Notch4 antibodies or antigen-binding fragments thereof (or the polynucleotide encoding the antibody or antigen-binding fragment) can also be formulated as a depot preparation. Such long acting formulations can be administered by implantation (for example subcutaneously or intramuscularly) or by intramuscular injection. Thus, for example, the antibodies can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the Notch4 antibodies can be formulated into ointments, salves, gels, or creams as generally known in the art. A wash solution can be used locally to treat an injury or inflammation to accelerate healing.

The compositions can, if desired, be presented in a pack or dispenser device which can contain one or more-unit dosage forms containing the active ingredient. The pack can for example comprise metal or plastic foil, such as a blister pack. The pack or dispenser device can be accompanied by instructions for administration.

### Dosage and Administration

The method and compositions provided herein can be used to treat a disease or disorder associated with an increased level of Notch4, as compared to a Notch4 level in a non-disease or non-disordered state, in a subject by administering a therapeutically effective amount of an antibody or antigen-binding fragment thereof that binds Notch4. In one embodiment, the subject can be a mammal. In another embodiment, the mammal can be a human, although the approach is effective with respect to all mammals.

The appropriate dosage range for a Notch4 antibody (or antigen-binding fragment thereof) depends upon the potency of the agent, and includes amounts large enough to produce the desired effect, e.g., reduction in at least one sign or symptom of a disease or disorder associated with an increased level of Notch4. The dosage should not be so large as to cause unacceptable or life-threatening adverse side effects. Generally, the dosage will vary with the type of antibody or antigen-binding fragment, and with the age, condition, and sex of the patient. The dosage can be determined by one of skill in the art and can also be adjusted by the individual physician in the event of any complication.

The effective amount can be based upon, among other things, the specificity of the antibody, the size of the antibody, the biodegradability of the antibody, the bioactivity and/or the bioavailability of the antibody or antigen binding fragment. For example, if the antibody does not degrade quickly, is bioavailable and highly active, a smaller amount will be required to be effective. One of skill in the art can routinely perform empirical activity tests to determine the bioactivity in bioassays and thus determine the effective amount.

Typically, the dosage ranges for a Notch4 antibody or fragment thereof are in the range of 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 0.5 μg/mL and 30 μg/mL. Dosages for monoclonal antibodies can also be based on rational dosage predictions such as those described in Bai et al. “A Guide to Rational Dosing of Monoclonal Antibodies” Clinical Pharmacokinetics 51:119-135 (2012).

As one of skill in the art will appreciate, the dosage of an anti-Notch4 antibody or antigen-binding fragment can vary depending upon the dosage form employed and the route of administration utilized. Compositions, methods, and uses that exhibit large therapeutic indices (i.e., the dose ration between toxic and therapeutic effects) are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. A therapeutically effective amount is an amount of an anti-Notch4 agent that is sufficient to produce a statistically significant, measurable change of a given symptom of an disease or disorder (see “Efficacy Measurement” below). Such effective amounts can also be gauged in clinical trials as well as animal studies for a given agent.

An appropriate therapeutic amount or dose for treating a human subject can be informed by data collected in cell cultures or animal models. In some embodiments, the therapeutic efficacy can be estimated by the ED50 in an animal model (the dose therapeutically effective in 50% of the population). Murine models of a disease or disorder associated with increased levels of Notch4 (e.g., asthma) can be used, in part, to inform one of skill in the art with respect to potency of a given antibody, the dosage range needed for efficacy, and toxic doses.

Therapeutic compositions can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

Administration of the doses recited above or as employed by a skilled clinician can be repeated for a limited and defined period of time. In some embodiments, the doses are given once a day, or multiple times a day (e.g., at least two times a day, at least three times a day etc). In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose. In one embodiment of the methods described herein, the anti-Notch4 agent is administered at least once per day. In one embodiment, the anti-Notch4 agent is administered daily. In one embodiment, the Notch4 antibody or antigen-binding fragment thereof is administered every other day. In one embodiment, the anti-Notch4 agent is administered every 6 to 8 days. In one embodiment, the agent is administered weekly. In one embodiment, the agent is administered monthly. In one embodiment, the agent is administered annually.

Administration of an antibody or fragment thereof that binds Notch4 can be constant for a certain period of time or periodic and at specific intervals. The anti-Notch4 agent can be delivered hourly, daily, weekly, monthly, yearly (e.g. in a time release form) or as a one-time delivery. The delivery can be continuous delivery for a period of time, e.g. intravenous delivery.

The agents described herein can be administered to a subject in need thereof by any appropriate route which results in an effective treatment in the subject. For example, agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

In some embodiments, the Notch4 antibody or antigen-binding fragment thereof can be administered to a subject by any mode of administration that delivers the agent systemically or locally to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the agents described herein, other than directly into a target site, tissue, or organ, such that it enters the subject's circulatory system and, thus, is subject to metabolism and other like processes.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology, if so desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

Combinational Therapy

In one embodiment, the antibody or antibody reagent described herein is used as a monotherapy. In one embodiment, the antibody or antibody reagent described herein can be used in combination with other known agents and therapies for a disease or disorder associated with an increased level of Notch4. Administered “in combination,” as used herein, means that two (or more) different treatments are delivered to the subject during the course of the subject's affliction with the disorder, e.g., the two or more treatments are delivered after the subject has been diagnosed with the disorder or disease and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat asthma include, but are not limited to, inhaled corticosteroids (e.g., fluticasone (Flonase, Flovent HFA), budesonide (Pulmicort Flexhaler, Rhinocort), flunisolide (Aerospan HFA), ciclesonide (Alvesco, Omnaris, Zetonna), beclomethasone (Qnasl, Qvar), mometasone (Asmanex) and leukotriene modifiers (e.g., montelukast (Singulair), zafirlukast (Accolate) and zileuton (Zyflo)); long-acting beta agonists (e.g., salmeterol (Serevent) and formoterol (Foradil, Perforomist)); combination inhalers (e.g., fluticasone-salmeterol (Advair Diskus), budesonide-formoterol (Symbicort) and formoterol-mometasone (Dulera)); theophylline (e.g., Theophylline (Theo-24, Elixophylline)); short-acting beta agonists (e.g., albuterol (ProAir HFA, Ventolin HFA, others) and levalbuterol (Xopenex)); ipratropium (e.g., Atrovent); and oral and intravenous corticosteroids.

Exemplary therapeutics used to treat an allergic disease include, but are not limited to, anti-inflammatory therapeutics (e.g., corticosteroids, glucocorticoids, or mineralcorticoids); antihistamines (e.g., Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); and adrenaline.

Exemplary treatments for COVID-19 are designed to treat individual symptoms, for example, a corticosteroid inhaler is prescribed to a subject having difficulty breathing, or a ventilator can be used for a subject having a serious illness related to COVID-19. Plasma from a subject that has been previously diagnosed with having COVID-19, and has recovered from COVID-19, has been administered as a COVID-19 therapeutic.

In one embodiment, the additional therapeutic is an anti-viral. Exemplary anti-virals include, but are not limited to, Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza®), Biktarvy Boceprevir (Victrelis®), Cidofovir, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Maraviroc, Methisazone, Moroxydine, Nelfinavir, Nevirapine, Nexavir®, Nitazoxanide, Norvir, Nucleoside analogues, Oseltamivir (Tamiflu®), Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

### Efficacy

The efficacy of a given treatment for a disease or disorder associated with an increased level of Notch4 can be determined by the skilled clinician. However, a treatment is considered “effective treatment,” as the term is used herein, if any one or all of the signs or symptoms of the disease/disorder is/are altered in a beneficial manner, or other clinically accepted symptoms or markers of disease are improved, or ameliorated, e.g., by at least 10% following treatment with a Notch4 antibody or antigen binding fragment thereof. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or disorder; or (2) relieving the disease, e.g., causing regression of symptoms; and (3) preventing or reducing the likelihood of the development of the disease, or preventing secondary diseases/disorders associated with the disease or disorder.

An effective amount for the treatment of a disease or disorder means that amount which, when administered to a mammal in need thereof, is sufficient to result in effective treatment as that term is defined herein, for that disease. Efficacy of an agent can be determined by assessing physical indicators of the disease or disorder.

Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example animal models of systemic lupus erythematosis, e.g. a murine model. When using an experimental animal model, efficacy of treatment is evidenced when a statistically significant change in a marker is observed.

### Kits

A Notch4 binding protein described herein can be provided in a kit, e.g., as a component of a kit. For example, the kit includes (a) an antibody, an antigen fragment or a polynucleotide described herein, and, optionally (b) informational material. The informational material can be descriptive, instructional, marketing or other material that relates to the methods described herein and/or the use of an antibody, an antigen fragment or a polynucleotide described herein for the methods described herein.

The informational material of the kits is not limited in its form. In one embodiment, the informational material can include information about production of the antibody, antigen binding fragment or the polynucleotide encoding the antibody or the antigen binding fragment, their molecular weight, concentration, date of expiration, batch or production site information, and so forth. In one embodiment, the informational material relates to using the antibody, antigen binding fragment or the polynucleotide to treat, prevent, or diagnosis of disorders and conditions, e.g., diseases or disorders associated with elevated levels of Notch4.

In one embodiment, the informational material can include instructions to administer the antibody, the antigen binding fragment or the polynucleotide in a suitable manner to perform the methods described herein, e.g., in a suitable dose, dosage form, or mode of administration (e.g., a dose, dosage form, or mode of administration described herein). In another embodiment, the informational material can include instructions to administer the antibody, the antigen binding fragment or the polynucleotide to a suitable subject, e.g., a human, e.g., a human having, or at risk for, a disorder or condition described herein, e.g., asthma.

The informational material of the kits is not limited in its form. In many cases, the informational material, e.g., instructions, is provided in print but can also be in other formats, such as computer readable material.

The antibody, antigen binding fragment or the polynucleotide can be provided in any form, e.g., liquid, dried or lyophilized form. It is preferred that the antibody, antigen binding fragment or the polynucleotide be substantially pure and/or sterile. When the antibody, antigen binding fragment or the polynucleotide is provided in a liquid solution, the liquid solution preferably is an aqueous solution, with a sterile aqueous solution being preferred. When the antibody, antigen binding fragment or the polynucleotide is provided as a dried form, reconstitution generally is by the addition of a suitable solvent. The solvent, e.g., sterile water or buffer, can optionally be provided in the kit.

The kit can include one or more containers for the composition containing the antibody, antigen binding fragment or the polynucleotide. In some embodiments, the kit contains separate containers, dividers or compartments for the composition and informational material. For example, the composition can be contained in a bottle, vial, or syringe, and the informational material can be contained association with the container. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an antibody or antigen binding fragment or a polynyucleotide. For example, the kit includes a plurality of syringes, ampules, foil packets, or blister packs, each containing a single unit dose of an antibody or antigen binding fragment or a polynyucleotide. The containers of the kits can be air tight, waterproof (e.g., impermeable to changes in moisture or evaporation), and/or light-tight.

The kit optionally includes a device suitable for administration of the composition, e.g., a syringe, inhalant, dropper (e.g., eye dropper), swab (e.g., a cotton swab or wooden swab), or any such delivery device. In one embodiment, the device is an implantable device that dispenses metered doses of the binding protein. The disclosure also features a method of providing a kit, e.g., by combining components described herein.

The invention disclosed herein can be further described in any of the numbered paragraphs provided herein below.
1) An antibody or antigen binding fragment that binds to Notch4 and comprises:
   a. a heavy chain variable region (V_{H}) comprising an amino acid sequence having at least 85% identity to an amino acid sequence selected from the group consisting of SEQ ID NOs 2, 4, or 6; or
   b. a heavy chain variable region (V_{H}) comprising an amino acid sequence encoded by a nucleotide sequence having at least 85% identity to a nucleotide sequence selected from the group consisting of SEQ ID NOs 3, 5, or 7; or
   c. a light chain variable region (V_{L}) comprising an amino acid sequence having at least 85% sequence identity to an amino acid sequence selected from the group consisting of SEQ ID NOs 8, 10, 12 or 14; or
   d. a light chain variable region (V_{L}) comprising an amino acid sequence encoded by a nucleotide sequence having at least 85% identity to a nucleotide sequence selected from the group consisting of SEQ ID NOs 9, 11, 13, or 15.
2) The antibody or antigen binding fragment of any preceding paragraph, wherein the antibody or antigen-binding fragment comprises a nanobody, an scFv, a monoclonal antibody, a humanized antibody, a human antibody, a recombinant antibody, a chimeric antibody, or a Fab fragment.
3) The antibody or antigen binding fragment of any preceding paragraph, wherein the heavy chain variable region comprises a complementarity determining region 1 (CDR_H1) comprising an amino acid sequence that differs by no more than four amino acids from an amino acid sequence selected from the group consisting of: SEQ ID NOs.: 16, 22, 28, and 34.
4) The antibody or antigen binding fragment of any preceding paragraph, wherein the heavy chain variable region comprises a complementarity determining region 1 (CDR_H2) comprising an amino acid sequence that differs by no more than four amino acids from SEQ ID NO: 17, 23, 29, and 35.
5) The antibody or antigen binding fragment of any preceding paragraph, wherein the heavy chain variable region comprises a complementarity determining region 1 (CDR_H3) comprising an amino acid sequence that differs by no more than four amino acids from an amino acid sequence selected from the group consisting of: SEQ ID NOs.: 18, 24, 30, and 36.
6) The antibody or antigen binding fragment of any preceding paragraph, wherein the light chain variable region comprises a complementarity determining region 1 (CDR_L1) comprising an amino acid sequence that differs by no more than four amino acids from an amino acid sequence selected from the group consisting of: SEQ ID NOs.: SEQ ID NOs: 19, 25, 31, and 37.
7) The antibody or antigen binding fragment of any preceding paragraph, wherein the light chain variable region comprises a complementarity determining region 1 (CDR_L2) comprising an amino acid sequence that differs by no more than two amino acids from an amino acid sequence selected from the group consisting of: SEQ ID NOs.: 20, 26, 32, and 38.
8) The antibody or antigen binding fragment of any preceding paragraph, wherein the light chain variable region comprises a complementarity determining region 1 (CDR_L3) comprising an amino acid sequence that differs by no more than four amino acids from an amino acid sequence selected from the group consisting of: SEQ ID NOs.: 21, 27, 33, and 39.
9) The antibody or antigen binding fragment of any preceding paragraph, wherein the heavy chain variable region comprises a CDR_H1 comprising an amino acid sequence that differs by no more than four amino acids from SEQ ID NO: 16, a CDR_H2 comprising an amino acid sequence that differs by no more than four amino acids from SEQ ID NO: 17, and a CDR_H3 comprising an amino acid sequence that differs by no more than four amino acids from SEQ ID NO: 18.
10) The antibody or antigen binding fragment of any preceding paragraph, wherein the heavy chain variable region comprises a CDR_H1 comprising an amino acid sequence that differs by no more than four amino acids from SEQ ID NO: 22, a CDR_H2 comprising an amino acid sequence that differs by no more than four amino acids from SEQ ID NO: 23, and a CDR_H3 comprising an amino acid sequence that differs by no more than four amino acids from SEQ ID NO: 24.
11) The antibody or antigen binding fragment of any preceding paragraph, wherein the heavy chain variable region comprises a CDR_H1 comprising an amino acid sequence that differs by no more than four amino acids from SEQ ID NO: 28, a CDR_H2 comprising an amino acid sequence that differs by no more than four amino acids from SEQ ID NO: 29, and a CDR_H3 comprising an amino acid sequence that differs by no more than four amino acids from SEQ ID NO: 30.
12) The antibody or antigen binding fragment of any preceding paragraph, wherein the heavy chain variable region comprises a CDR_H1 comprising an amino acid sequence that differs by no more than four amino acids from SEQ ID NO: 34, a CDR_H2 comprising an amino acid sequence that differs by no more than four amino acids from SEQ ID NO: 35, and a CDR_H3 comprising an amino acid sequence that differs by no more than four amino acids from SEQ ID NO: 36.
13) The antibody or antigen binding fragment of any preceding paragraph, wherein the light chain variable region comprises a CDR_L1 comprising an amino acid sequence that differs by no more than four amino acids from SEQ ID NO: 19, a CDR_L2 comprising an amino acid sequence that differs by no more than four amino acids from SEQ ID NO: 20, and a CDR_L3 comprising an amino acid sequence that differs by no more than four amino acids from SEQ ID NO: 21.
14) The antibody or antigen binding fragment of any preceding paragraph, wherein the light chain variable region comprises a CDR_L1 comprising an amino acid sequence that differs by no more than four amino acids from SEQ ID NO: 25, a CDR_L2 comprising an amino acid sequence that differs by no more than four amino acids from SEQ ID NO: 26, and a CDR_L3 comprising an amino acid sequence that differs by no more than four amino acids from SEQ ID NO: 27.
15) The antibody or antigen binding fragment of any preceding paragraph, wherein the light chain variable region comprises a CDR_L1 comprising an amino acid sequence that differs by no more than four amino acids from SEQ ID NO: 31, a CDR_L2 comprising an amino acid sequence that differs by no more than four amino acids from SEQ ID NO: 32, and a CDR_L3 comprising an amino acid sequence that differs by no more than four amino acids from SEQ ID NO: 33.
16) The antibody or antigen binding fragment of any preceding paragraph, wherein the light chain variable region comprises a CDR_L1 comprising an amino acid sequence that differs by no more than four amino acids from SEQ ID NO: 37, a CDR_L2 comprising an amino acid sequence that differs by no more than four amino acids from SEQ ID NO: 38, and a CDR_L3 comprising an amino acid sequence that differs by no more than four amino acids from SEQ ID NO: 39.
17) The antibody or antigen binding fragment of any preceding paragraph, wherein the heavy chain variable region comprises an amino acid sequence having at least 90% identity to an amino acid sequence selected from the group consisting of SEQ ID NOs: 2, 4, or 6.
18) The antibody or antigen binding fragment of any preceding paragraph, wherein the heavy chain variable region comprises an amino acid sequence encoded by a nucleotide sequence having at least 90% identity to a nucleotide sequence selected from the group consisting of SEQ ID NOs: 3, 5, or 7.
19) The antibody or antigen binding fragment of any preceding paragraph, wherein the light chain variable region comprises an amino acid sequence having at least 90% identity to an amino acid sequence selected from the group consisting of SEQ ID NOs: 8, 10, 12 or 14.
20) The antibody or antigen binding fragment of any preceding paragraph, wherein the light chain variable region comprises an amino acid sequence encoded by a nucleotide sequence having at least 90% identity to a nucleotide sequence selected from the group consisting of SEQ ID NOs: 9, 11, 13, or 15.
21) The antibody or antigen binding fragment of any preceding paragraph, wherein the antibody or antigen binding fragment is a Notch4 inhibitor or a Notch4 neutralizing antibody.
22) The antibody or antigen binding fragment of any preceding paragraph, wherein the antibody or antigen binding fragment reduces Notch4 activity by at least 20% as compared to Notch4 activity in the absence of the antibody or antigen-binding fragment.
23) The antibody or antigen binding fragment of any preceding paragraph, wherein the antibody or antigen binding fragment disrupts binding of Notch4 to its cognate ligand.
24) An antibody or antigen binding fragment that binds to Notch4 and comprises:
   a. a heavy chain variable region (V_{H}) comprising:
      i. a complementarity determining region 1 (CDR_H1) sequence comprising an amino acid sequence that differs by no more than four amino acids from an amino acid sequence selected from the group consisting of SEQ ID NOs 16, 22, 28, and 34; or
      ii. a complementarity determining region 2 (CDR_H2) sequence comprising an amino acid sequence that differs by no more than four amino acids from an amino acid sequence selected from the group consisting of SEQ ID NOs 17, 23, 29, and 35; or
      iii. a complementarity determining region 3 (CDR_H3) sequence comprising an amino acid sequence that differs by no more than four amino acids from an amino acid sequence selected from the group consisting of SEQ ID NOs 18, 24, 30, and 36; or
   b. a light chain variable region (V_{L}) comprising:
      i. a complementarity determining region 1 (CDR_L1) sequence comprising an amino acid sequence that differs by no more than four amino acids from an amino acid sequence selected from the group consisting of SEQ ID NOs 19, 25, 31, and 37; or
      ii. a complementarity determining region 2 (CDR_L2) sequence comprising an amino acid sequence that differs by no more than two amino acids from an amino acid sequence selected from the group consisting of SEQ ID NOs 20, 26, 32, and 38; or
      iii. a complementarity determining region 3 (CDR_L3) sequence comprising an amino acid sequence that differs by no more than four amino acids from an amino acid sequence selected from the group consisting of SEQ ID NOs 21, 27, 33, and 39.
25) A composition comprising an antibody or antigen binding fragment of any preceding paragraph.
26) The composition of any preceding paragraph, wherein the composition comprises a pharmaceutically acceptable excipient or carrier.
27) A cell comprising an antibody or antigen binding fragment of any preceding paragraph.
28) A polynucleotide comprising a nucleotide sequence encoding an antibody or antigen binding fragment of any preceding paragraph.
29) The polynucleotide of any preceding paragraph, wherein the polynucleotide is comprised in a vector.
30) A composition comprising a polynucleotide of any preceding paragraph.
31) A cell comprising a polynucleotide of any preceding paragraph.
32) A kit comprising:
   a. an antibody or antigen binding fragment of any preceding paragraph;
   b. a polynucleotide of any preceding paragraph;
   c. a composition of any preceding paragraph; or
   d. a cell of any preceding paragraph.
33) The kit of any preceding paragraph, further comprising instructions for use thereof
34) A method for reducing Notch4 activity, the method comprising: administering an antibody or antigen binding fragment of any preceding paragraph, a polynucleotide of any preceding paragraph, or a composition of any preceding paragraph to a cell.
35) The method of any preceding paragraph, wherein said administering to the cell is in vitro.
36) The method of any preceding paragraph, wherein said administering to the cell is in vivo.
37) The method of any preceding paragraph, wherein said administering to the cell is in a subject.
38) A method for reducing at least one symptom of a disease or disorder associated with increased levels of Notch4, the method comprising administering a therapeutically effective amount of an antibody or antigen binding fragment of any preceding paragraph, a polynucleotide of any preceding paragraph, or a composition of any preceding paragraph to a subject in need thereof.
39) The method of any preceding paragraph, wherein the disease or disorder associated with an increased level of Notch4 is selected from the group consisting of asthma, allergic disease, and a disease or condition associated with pulmonary viral infection.
40) The method of any preceding paragraph, further comprising, prior to administering, a step of diagnosing the subject with the disease or disorder associated with an increased level of Notch4.
41) The method of any preceding paragraph, wherein the subject is a mammal.
42) The method of any preceding paragraph, wherein the subject is human.
43) The antibody or antigen binding fragment of any preceding paragraph wherein the heavy chain variable region comprises an amino acid sequence having at least 91%, at least 92%, at least 93%, at least 94%, at least 95%, at least 96%, at least 97%, at least 98%, or at least 99% identity to an amino acid sequence selected from the group consisting of SEQ ID NOs: 2, 4, or 6.
44) The antibody or antigen binding fragment of any preceding paragraph, wherein the heavy chain variable region comprises an amino acid sequence selected from the group consisting of SEQ ID NOs: 2, 4, or 6.
45) The antibody or antigen binding fragment of any preceding paragraph, wherein the heavy chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 3, 5, or 7.
46) The antibody or antigen-binding fragment of any preceding paragraph, wherein the heavy chain variable region comprises an amino acid sequence encoded by a nucleotide sequence selected from the group consisting of SEQ ID NOS: 3, 5, or 7.
47) The antibody or antigen binding fragment of any preceding paragraph, wherein the light chain variable region comprises an amino acid sequence having at least 91%, at least 92%, at least 93%, at least 94%, at least 95%, at least 96%, at least 97%, at least 98%, or at least 99% identity to an amino acid sequence selected from the group consisting of SEQ ID NOs: 8, 10, 12 or 14.
48) The antibody or antigen binding fragment of any preceding paragraph, wherein the light chain variable region comprises an amino acid sequence selected from the group consisting of SEQ ID NOs: 8, 10, 12, or 14.
49) The antibody or antigen binding fragment of any preceding paragraph, wherein the light chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 9, 11, 13, or 15.
50) The antibody or antigen binding fragment of any preceding paragraph, wherein the light chain variable region comprises an amino acid sequence encoded by a nucleotide sequence selected from the group consisting of SEQ ID NOs: 9, 11, 13, or 15.
51) The antibody or antigen-binding fragment of any preceding paragraph, comprising
   a. a heavy chain variable region (V_{H}) comprising:
      i. a complementarity determining region 1 (CDR_H1) sequence comprising an amino acid sequence that differs by no more than four amino acids from an amino acid sequence selected from the group consisting of SEQ ID NOs 16, 22, 28, and 34;
      ii. a complementarity determining region 2 (CDR_H2) sequence comprising an amino acid sequence that differs by no more than four amino acids from an amino acid sequence selected from the group consisting of SEQ ID NOs 17, 23, 29, and 35;
      iii. a complementarity determining region 3 (CDR_H3) sequence comprising an amino acid sequence that differs by no more than four amino acids from an amino acid sequence selected from the group consisting of SEQ ID NOs 18, 24, 30, and 36; and
   b. a light chain variable region (V_{L}) comprising:
      i. a complementarity determining region 1 (CDR_L1) sequence comprising an amino acid sequence that differs by no more than four amino acids from an amino acid sequence selected from the group consisting of SEQ ID NOs 19, 25, 31, and 37;
      ii. a complementarity determining region 2 (CDR_L2) sequence comprising an amino acid sequence that differs by no more than two amino acids from an amino acid sequence selected from the group consisting of SEQ ID NOs 20, 26, 32, and 38;
      iii. a complementarity determining region 3 (CDR_L3) sequence comprising an amino acid sequence that differs by no more than four amino acids from an amino acid sequence selected from the group consisting of SEQ ID NOs 21, 27, 33, and 39.
52) The antibody of any preceding paragraph, wherein the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:
   a. a heavy chain variable region (V_{H}) comprising:
      i. a complementarity determining region 1 (CDR_H1) sequence comprising an amino acid sequence selected from the group consisting of SEQ ID NOs 16, 22, 28, and 34;
      ii. a complementarity determining region 2 (CDR_H2) sequence comprising an amino acid sequence selected from the group consisting of SEQ ID NOs 17, 23, 29, and 35;
      iii. a complementarity determining region 3 (CDR_H3) sequence comprising an amino acid sequence selected from the group consisting of SEQ ID NOs 18, 24, 30, and 36; and
   b. a light chain variable region (V_{L}) comprising:
      i. a complementarity determining region 1 (CDR_L1) sequence comprising an amino acid sequence selected from the group consisting of SEQ ID NOs 19, 25, 31, and 37;
      ii. a complementarity determining region 2 (CDR_L2) sequence comprising an amino acid sequence selected from the group consisting of SEQ ID NOs 20, 26, 32, and 38;
      iii. a complementarity determining region 3 (CDR_L3) sequence comprising an amino acid sequence selected from the group consisting of SEQ ID NOs 21, 27, 33, and 39.
      iv.
53) The antibody of any preceding paragraph, wherein the antibody or antigen binding fragment thereof comprise:
   a. a heavy chain variable region (V_{H}) comprising:
      i. a complementarity determining region 1 (CDR_H1) sequence comprising an amino acid sequence selected from the group consisting of SEQ ID NOs 16, 22, 28, and 34;
      ii. a complementarity determining region 2 (CDR_H2) sequence comprising an amino acid sequence selected from the group consisting of SEQ ID NOs 17, 23, 29, and 35;
      iii. a complementarity determining region 3 (CDR_H3) sequence comprising an amino acid sequence selected from the group consisting of SEQ ID NOs 18, 24, 30, and 36; and
   b. a light chain variable region (V_{L}) comprising:
      i. a complementarity determining region 1 (CDR_L1) sequence comprising an amino acid sequence selected from the group consisting of SEQ ID NOs 19, 25, 31, and 37;
      ii. a complementarity determining region 2 (CDR_L2) sequence comprising an amino acid sequence selected from the group consisting of SEQ ID NOs 20, 26, 32, and 38;
      iii. a complementarity determining region 3 (CDR_L3) sequence comprising an amino acid sequence selected from the group consisting of SEQ ID NOs 21, 27, 33, and 39.
54) The method of any preceding paragraph, wherein the at least one symptom comprises tissue inflammation.
55) The method of any preceding paragraph, wherein the at least one symptom comprises a symptom selected from the group consisting of macrophage skewing, neutrophil infiltration, production of an inflammatory cytokine, and combinations thereof
56) The method of any preceding paragraph, wherein the inflammatory cytokine is IL-6 or IFNγ.
57) The antibody or antigen-binding fragment of any preceding paragraph, comprising a heavy chain variable region (V_{H}) and a light chain variable region (V_{L}),
   wherein the heavy chain variable region comprises complementarity determining regions CDR_H1, CDR_H2, and CDR_H3,
   wherein the light chain variable region comprises CDR_L1, CDR_L2, and CDR_L3, and wherein
   (a) (i) CDR_H1 comprises an amino acid sequence that differs by no more than two amino acid residues from that of SEQ ID NO: 16,
      (ii) CDR_H2 comprises an amino acid sequence that differs by no more than two amino acid residues from that of SEQ ID NO: 17,
      (iii) CDR_H3 comprises an amino acid sequence that differs by no more than two amino acid residues from that of SEQ ID NO: 18,
      (iv) CDR_L1 comprises an amino acid sequence that differs by no more than two amino acid residues from that of SEQ ID NO: 19,
      (v) CDR_L2 comprises an amino acid sequence that differs by no more than two amino acid residues from that of SEQ ID NO: 20; and
      (vi) CDR_L3 comprises an amino acid sequence that differs by no more than two amino acid residues from that of SEQ ID NO: 21; or
   (b) (i) CDR_H1 comprises an amino acid sequence that differs by no more than two amino acid residues from that of SEQ ID NO: 22,
      (ii) CDR_H2 comprises an amino acid sequence that differs by no more than two amino acid residues from that of SEQ ID NO: 23,
      (iii) CDR_H3 comprises an amino acid sequence that differs by no more than two amino acid residues from that of SEQ ID NO: 24,
      (iv) CDR_L1 comprises an amino acid sequence that differs by no more than two amino acid residues from that of SEQ ID NO: 25,
      (v) CDR_L2 comprises an amino acid sequence that differs by no more than two amino acid residues from that of SEQ ID NO: 26; and
      (vi) CDR_L3 comprises an amino acid sequence that differs by no more than two amino acid residues from that of SEQ ID NO: 27; or
   (c) (i) CDR_H1 comprises an amino acid sequence that differs by no more than two amino acid residues from that of SEQ ID NO: 28,
      (ii) CDR_H2 comprises an amino acid sequence that differs by no more than two amino acid residues from that of SEQ ID NO: 29,
      (iii) CDR_H3 comprises an amino acid sequence that differs by no more than two amino acid residues from that of SEQ ID NO: 30,
      (iv) CDR_L1 comprises an amino acid sequence that differs by no more than two amino acid residues from that of SEQ ID NO: 31,
      (v) CDR_L2 comprises an amino acid sequence that differs by no more than two amino acid residues from that of SEQ ID NO: 32; and
      (vi) CDR_L3 comprises an amino acid sequence that differs by no more than two amino acid residues from that of SEQ ID NO: 33; or
   (d) (i) CDR_H1 comprises an amino acid sequence that differs by no more than two amino acid residues from that of SEQ ID NO: 34,
      (ii) CDR_H2 comprises an amino acid sequence that differs by no more than two amino acid residues from that of SEQ ID NO: 35,
      (iii) CDR_H3 comprises an amino acid sequence that differs by no more than two amino acid residues from that of SEQ ID NO: 36,
      (iv) CDR_L1 comprises an amino acid sequence that differs by no more than two amino acid residues from that of SEQ ID NO: 37,
      (v) CDR_L2 comprises an amino acid sequence that differs by no more than two amino acid residues from that of SEQ ID NO: 38; and
      (vi) CDR_L3 comprises an amino acid sequence that differs by no more than two amino acid residues from that of SEQ ID NO: 39.
58) The antibody or antigen-binding fragment of any preceding paragraph, comprising: wherein
   (a) (i) CDR_H1 comprises an amino acid sequence of SEQ ID NO: 16,
      (ii) CDR_H2 comprises an amino acid sequence of SEQ ID NO: 17,
      (iii) CDR_H3 comprises an amino acid sequence of SEQ ID NO: 18,
      (iv) CDR_L1 comprises an amino acid sequence of SEQ ID NO: 19,
      (v) CDR_L2 comprises an amino acid sequence of SEQ ID NO: 20; and
      (vi) CDR_L3 comprises an amino acid sequence of SEQ ID NO: 21; or
   (b) (i) CDR_H1 comprises an amino acid sequence of SEQ ID NO: 22,
      (ii) CDR_H2 comprises an amino acid sequence of SEQ ID NO: 23,
      (iii) CDR_H3 comprises an amino acid sequence of SEQ ID NO: 24,
      (iv) CDR_L1 comprises an amino acid sequence of SEQ ID NO: 25,
      (v) CDR_L2 comprises an amino acid sequence of SEQ ID NO: 26; and
      (vi) CDR_L3 comprises an amino acid sequence of SEQ ID NO: 27; or
   (c) (i) CDR_H1 comprises an amino acid sequence of SEQ ID NO: 28,
      (ii) CDR_H2 comprises an amino acid sequence of SEQ ID NO: 29,
      (iii) CDR_H3 comprises an amino acid sequence of SEQ ID NO: 30,
      (iv) CDR_L1 comprises an amino acid sequence of SEQ ID NO: 31,
      (v) CDR_L2 comprises an amino acid sequence of SEQ ID NO: 32; and
      (vi) CDR_L3 comprises an amino acid sequence of SEQ ID NO: 33; or
   (d) (i) CDR_H1 comprises an amino acid sequence of SEQ ID NO: 34,
      (ii) CDR_H2 comprises an amino acid sequence of SEQ ID NO: 35,
      (iii) CDR_H3 comprises an amino acid sequence of SEQ ID NO: 36,
      (iv) CDR_L1 comprises an amino acid sequence of SEQ ID NO: 37,
      (v) CDR_L2 comprises an amino acid sequence of SEQ ID NO: 38; and
      (vi) CDR_L3 comprises an amino acid sequence of SEQ ID NO: 39.

### EXAMPLES

### Example 1

A cardinal feature of COVID-19 is lung inflammation and respiratory failure. In a prospective multi-country cohort of COVID-19 patients, we found that increased Notch4 expression on circulating regulatory T (Treg) cells was associated with disease severity, predicted mortality, and declined upon recovery. Deletion of Notch4 in Treg cells or therapy with anti-Notch4 antibodies in conventional and humanized mice normalized the dysregulated innate immunity and rescued disease morbidity and mortality induced by a synthetic analogue of viral RNA or by influenza H1N1 virus. Mechanistically, Notch4 suppressed the induction by interleukin-18 of amphiregulin, a cytokine necessary for tissue repair. Protection by Notch4 inhibition was recapitulated by therapy with Amphiregulin and, reciprocally, abrogated by its antagonism. Amphiregulin declined in COVID-19 subjects as a function of disease severity and Notch4 expression. Thus, Notch4 expression on Treg cells dynamically restrains amphiregulin-dependent tissue repair to promote severe lung inflammation, with therapeutic implications for COVID-19 and related infections.

### INTRODUCTION

The current pandemic caused by the severe acute respiratory syndrome coronavirus 2 (SARS-CoV-2) virus has resulted in massive morbidity and mortality figures both within the US and globally (Cucinotta and Vanelli, 2020). While many infected subjects are either asymptomatic or have a mild form of the disease, a subset suffers a more severe disease with pneumonia and marked hypoxia, leading to acute respiratory distress syndrome (Berlin et al., 2020; Richardson et al., 2020; Zhou et al., 2020a). In many of those patients, the disease proved fatal despite intensive respiratory support. Uncontrolled activation of the immune response, leading to a cytokine storm, is a key risk factor for mortality (Henderson et al., 2020; Lucas et al., 2020; Vabret et al., 2020). The molecular mechanisms governing lung disease severity are not well understood yet. Notably, innate immune cell hyperactivation plays a critical role in the pathogenesis of severe COVID-19 (Del Valle et al., 2020; Vabret et al., 2020; Zhou et al., 2020b), a finding also recapitulated in emerging mouse models (Winkler et al., 2020). Among the innate immune cytokines, interleukin (IL)-6 has been proposed to play a pathogenic role by virtue of its increased serum concentrations in severe COVID-19 subjects and reports of favorable clinical responses to anti-interleukin 6 receptor (IL-6R) monoclonal antibody (mAb) therapy (Copaescu et al., 2020; Investigators et al., 2021; Salama et al., 2021; Toniati et al., 2020; Xu et al., 2020). Other studies however have failed to confirm such a benefit (Hermine et al., 2021; Rosas et al., 2021; Salvarani et al., 2021; Veiga et al., 2021).

An IL-6-dependent pathway subverts lung regulatory T (Treg) cells to promote tissue inflammation in severe asthma by increasing Treg cell expression of the receptor Notch4 (Harb et al., 2020; Xia et al., 2018; Xia et al., 2015). In this setting, Notch4 acts via downstream pathways, including Hippo and Wnt, to disrupt Treg cell regulation of the T helper type 2 (Th2) and Th17 adaptive immune responses. In case studies, treatment of severe asthma patients with the anti-IL-6R mAb

Tocilizumab decreased Notch4 expression on circulating Treg cells and improved disease outcomes (Esty et al., 2019). The NOTCH4 locus is associated with critical illness in COVID-19 (Pairo-Castineira et al., 2021). Given this and the evidence supporting a pathogenic role for high levels of IL-6 in COVID-19, we examined the impact of inducible Notch4 expression on Treg cells in lung viral infection, including COVID-19. We found increased Notch4 expression in COVID-19 subjects as a function of disease severity. In mouse models of respiratory viral infections, Notch4 enabled virus-induced tissue inflammation by mechanisms distinct from those involved in its regulation of adaptive immunity in allergic airway inflammation. Rather, Notch4 expression inhibited Treg cell-mediated regulation of innate immune responses and promotion of tissue repair. The protective function of Notch4 inhibition involved increased production by Treg cells of the epidermal growth factor-like cytokine amphiregulin, which plays a critical role in in mediating tissue repair by Treg cells in lung viral infections (Arpaia et al., 2015). These findings identify Notch4 as an effector of COVID-19 disease severity, and point to interventions along the Notch4-amphiregulin axis as a viable therapeutic strategy to restore immune regulation in severe viral respiratory infections including COVID-19.

### Results

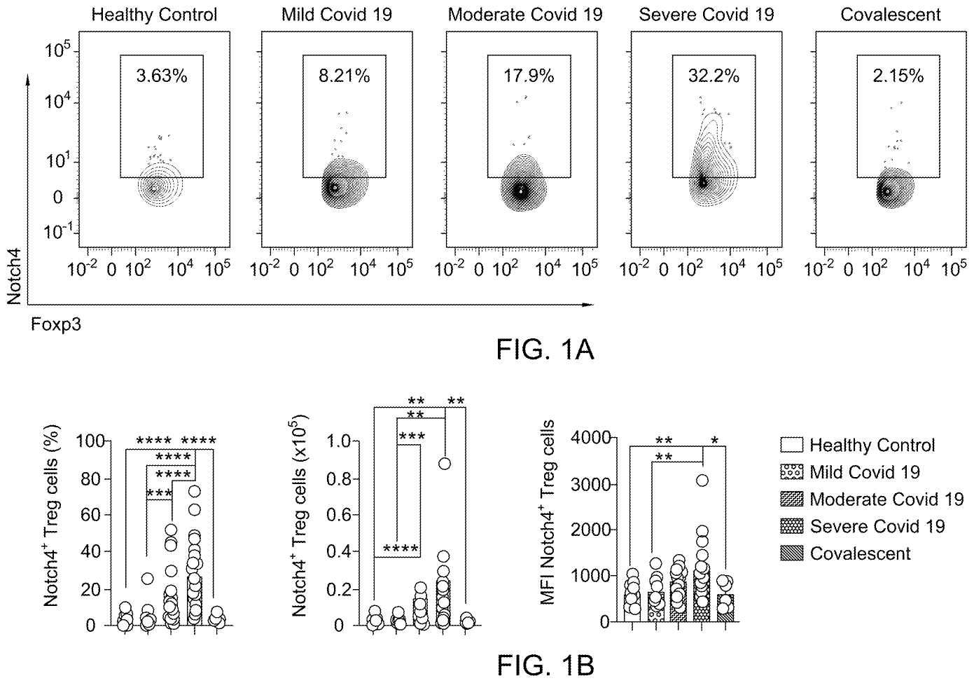

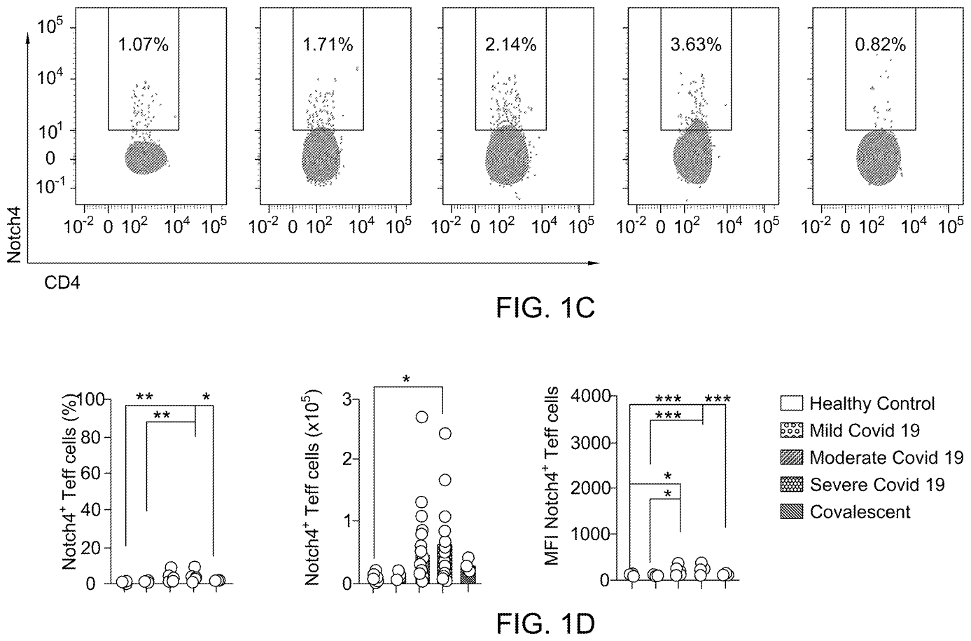

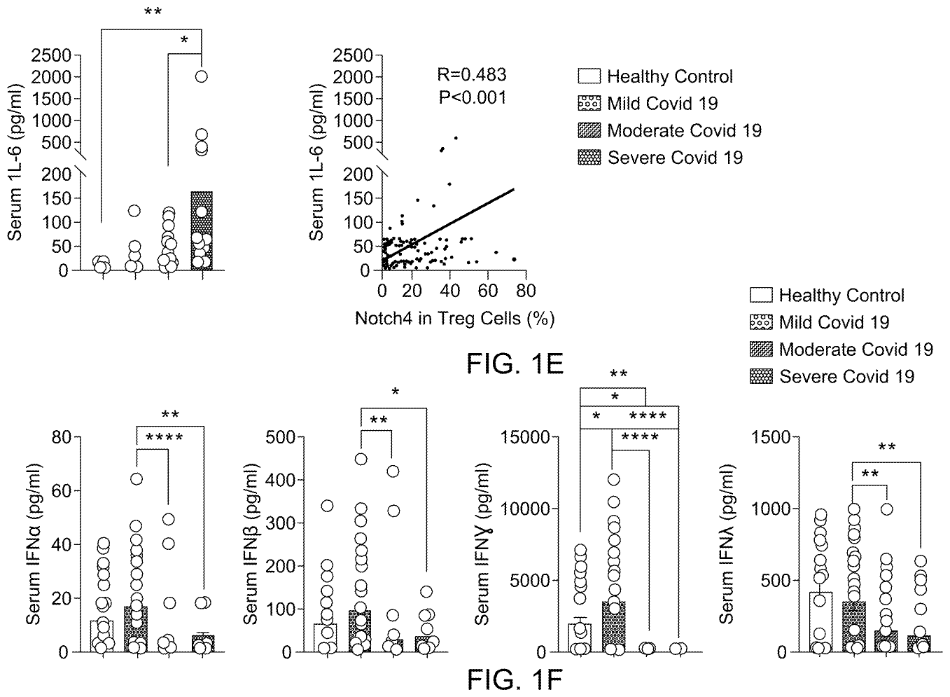

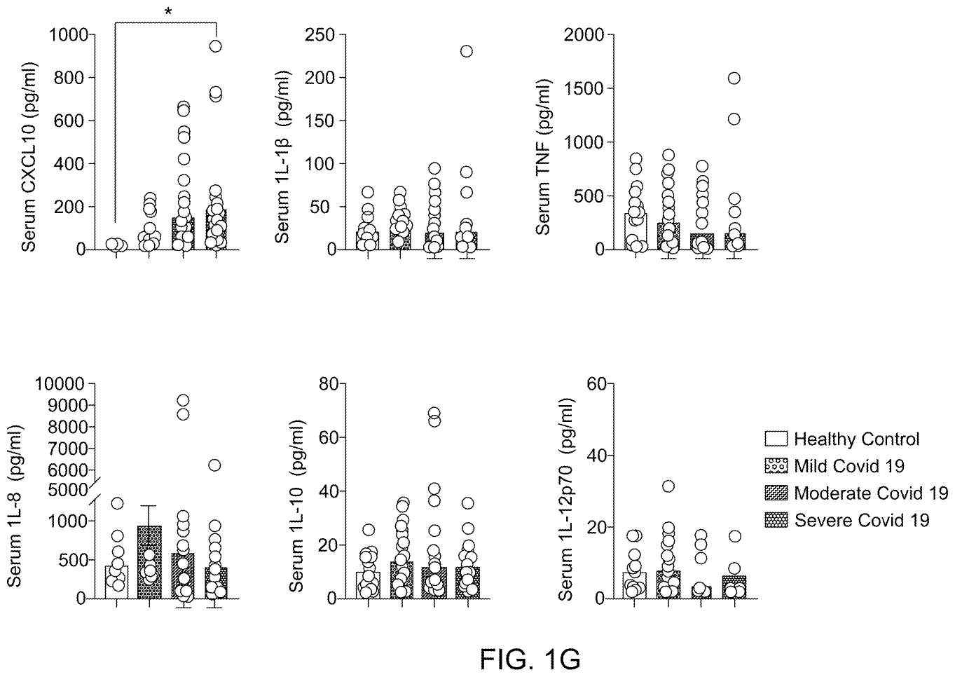

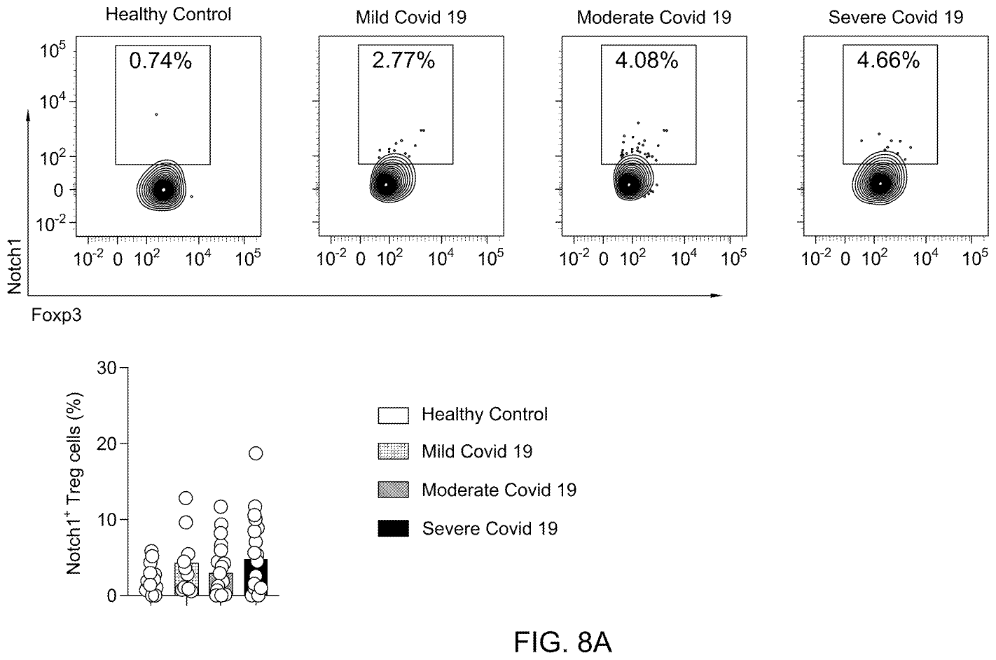

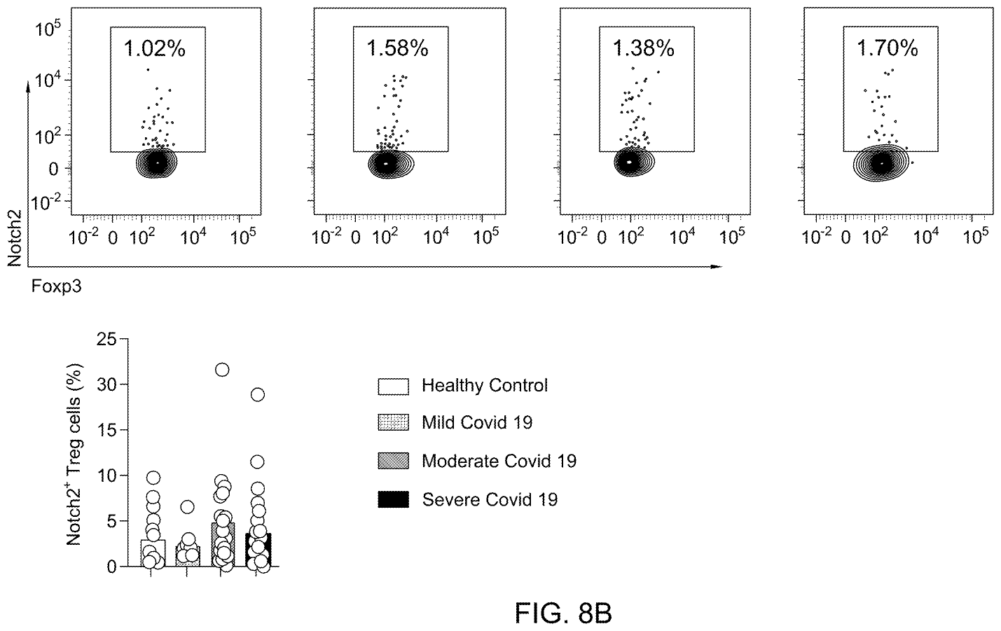

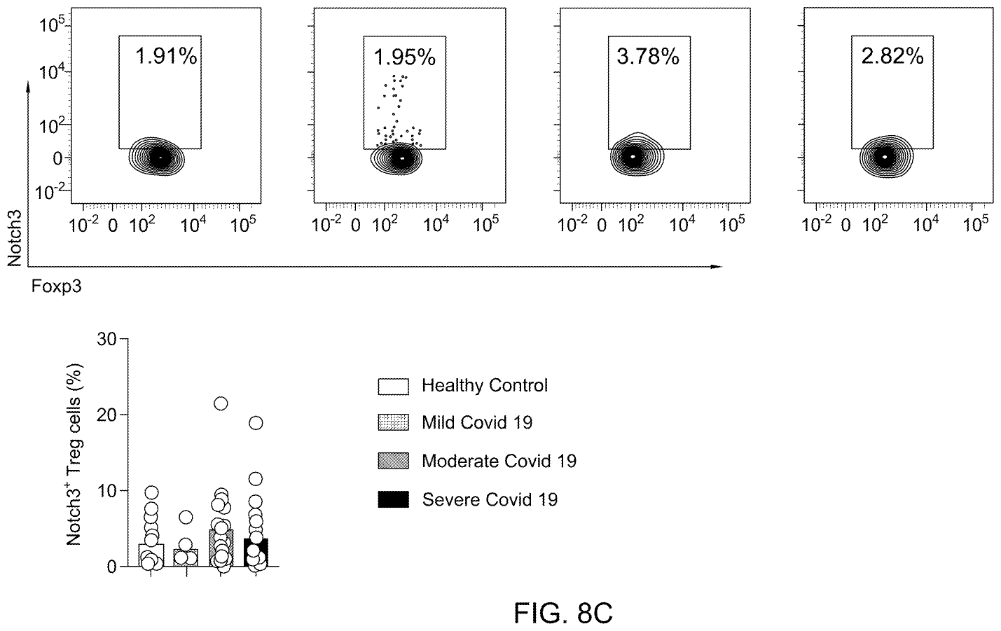

Treg cell Notch4 expression is independently predictive of mortality from COVID-19. To determine the association between Notch4 and disease severity in COVID-19 patients, we recruited three cohorts of patients with COVID-19 as well as healthy controls from Boston, Massachusetts (n=38 and n=10, respectively), Genoa, Italy (n=44 and n=10) and Istanbul, Turkey (n=36 and n=20) (Table S1). The patients were segregated into three disease severity groups (mild, moderate and severe) based on the need for hospitalization and advanced respiratory support, as well as a convalescent group following criteria detailed in the Methods section. Older male patients with a history of malignancy, cardiac disease, or endocrine disease had more severe disease (Table S1). Frequencies, absolute numbers and mean fluorescence intensity (MFI) of Notch4 expression on peripheral blood CD4+CD25+Foxp3+ Treg cells of COVID-19 subjects in the different cohorts progressively increased as a function of disease severity and declined precipitously in the convalescent subjects (FIG. 1A, 1B and Table S1). In contrast, there was a modest increase in the Notch4 expression on CD4+CD25−Foxp3− T effector (Teff) cells of subjects with moderate and severe disease, and otherwise minimal expression in the other subject groups (FIG. 1C, 1D). Also, there was no difference in the expression of the other Notch receptors including Notch1, Notch2 and Notch3 on circulating Treg cells amongst subjects in the different study groups (FIG. 8). In multivariate logistic regression adjusting for age, gender, comorbidities, glucocorticoid treatment, and serum IL-6 levels, each 1% increase in Notch4 expression in Treg cells was independently associated with a 1.046 higher odds of mortality (univariate odds ratio 1.055 [95% CI 1.008-1.090, p=0.023; multivariate odds ratio 1.057 [95% CI 1.01-1.11], p=0.017 (Table S1). Thus, for example, an increase in Treg cells expressing Notch4 by 10% in a 65-year-old male patient with a history of malignancy treated with steroids would lead to a rise in predicted mortality from 28.4% to 38.3%. Overall, serum IL-6 concentrations were positively correlated with Treg cell Notch4 expression (FIG. 1E). Moreover, the serum concentrations of interferon alpha (IFNα), IFNβ and IFNγ were decreased in the moderate and severe patients compared to mild patients (FIG. 1F) (Blanco-Melo et al., 2020; Hadjadj et al., 2020). In contrast, the serum concentrations of other cytokines including IL-1β, TNF, IL-8, IL-10, IL-12p70 were unchanged, while those of CXCL10 were slightly increased in the severe subjects (FIG. 1G).

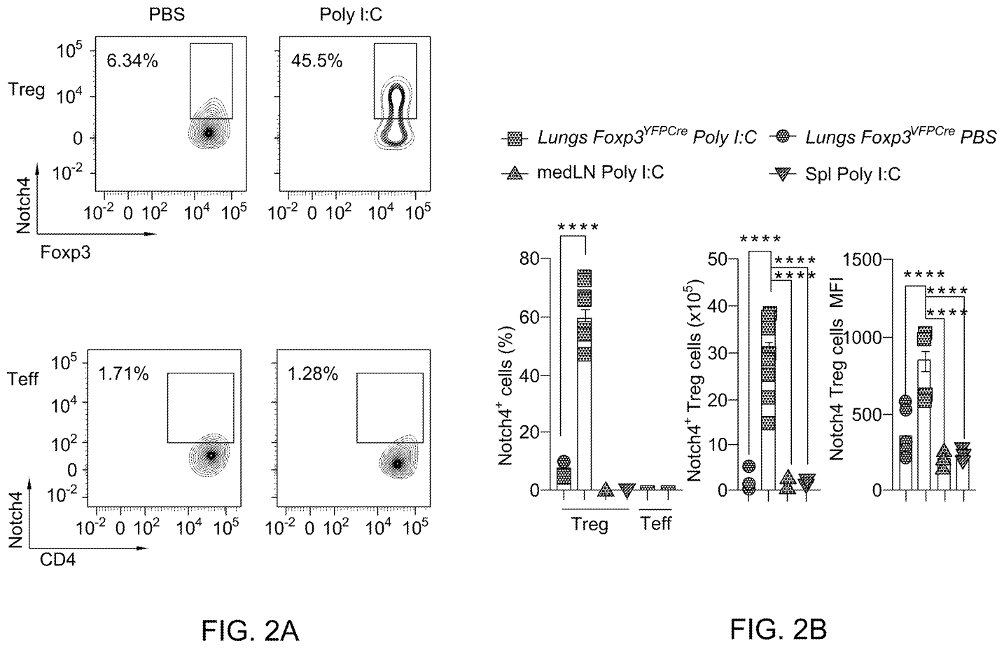

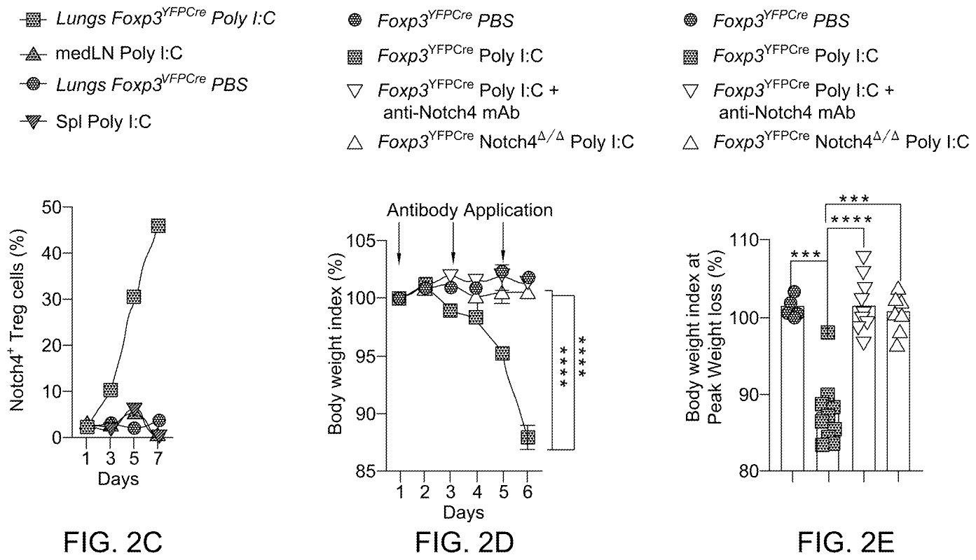

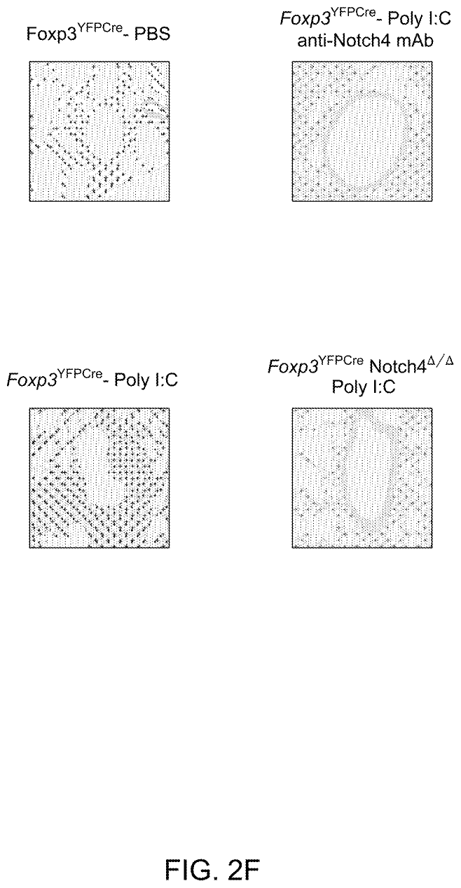

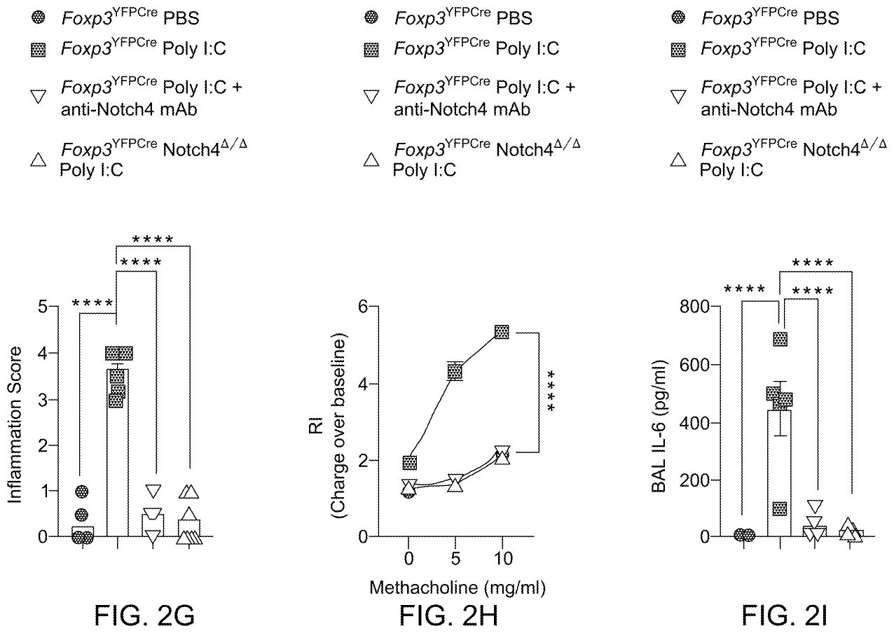

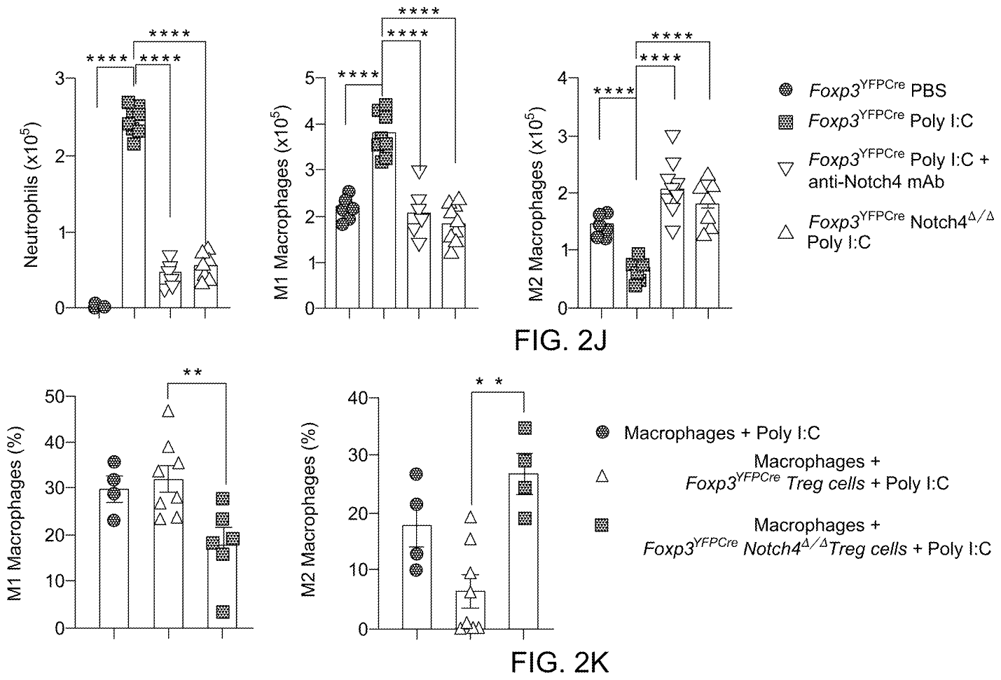

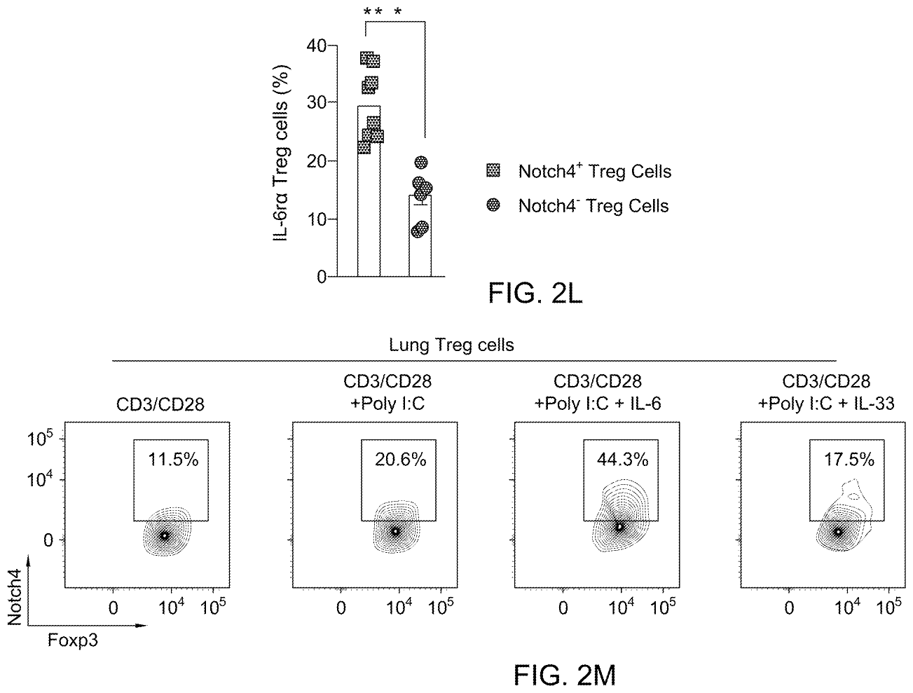

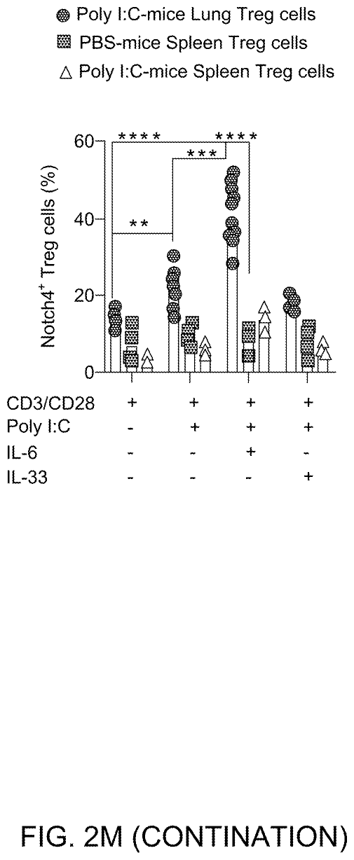

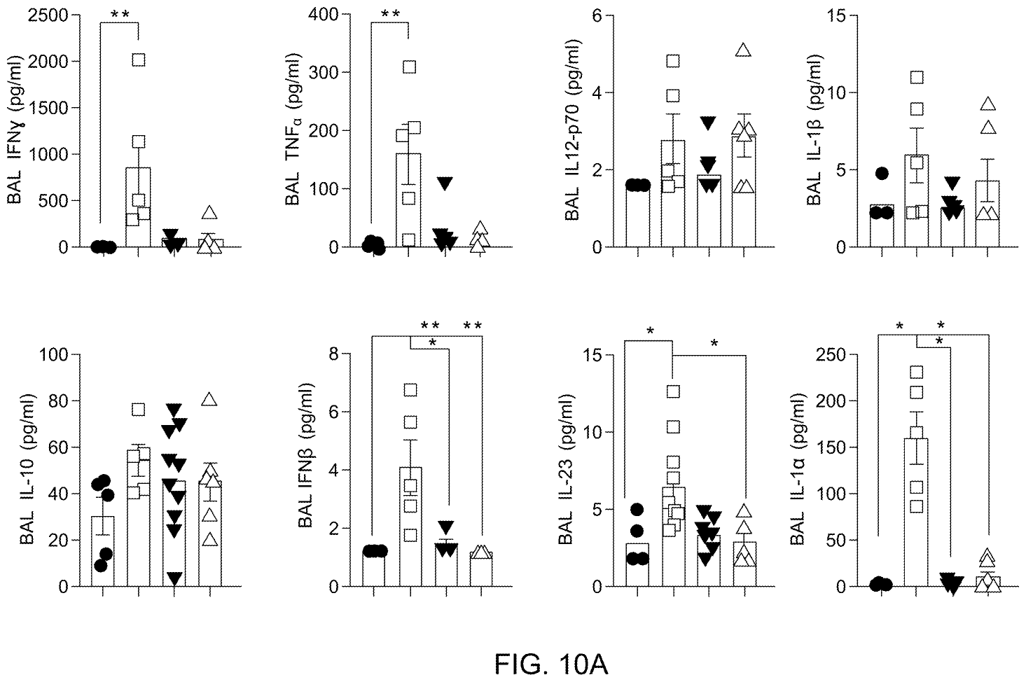

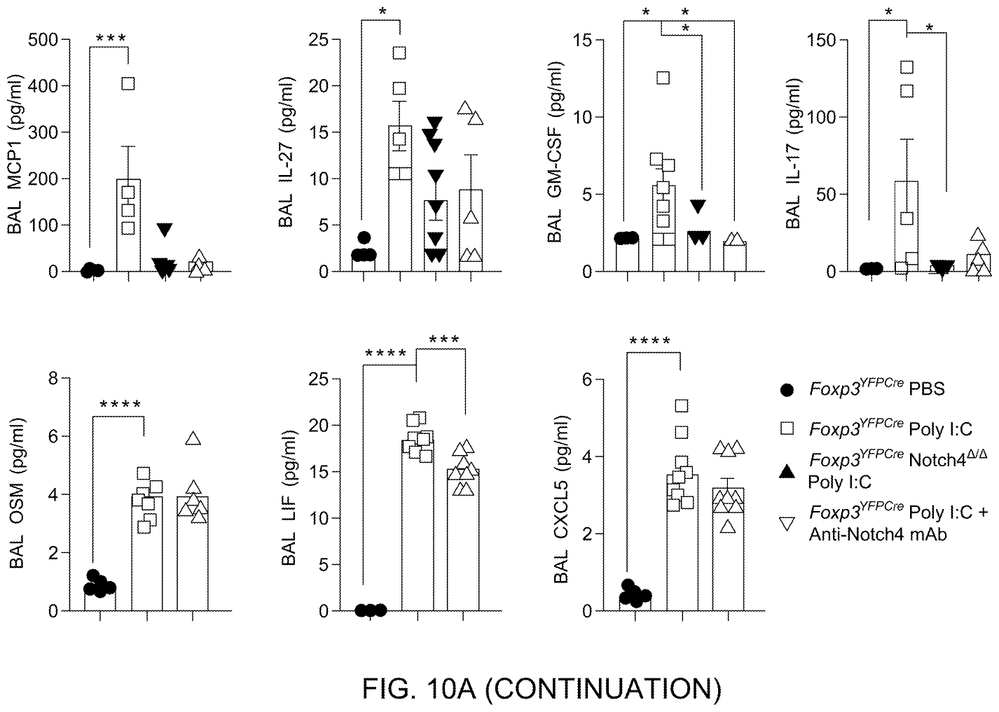

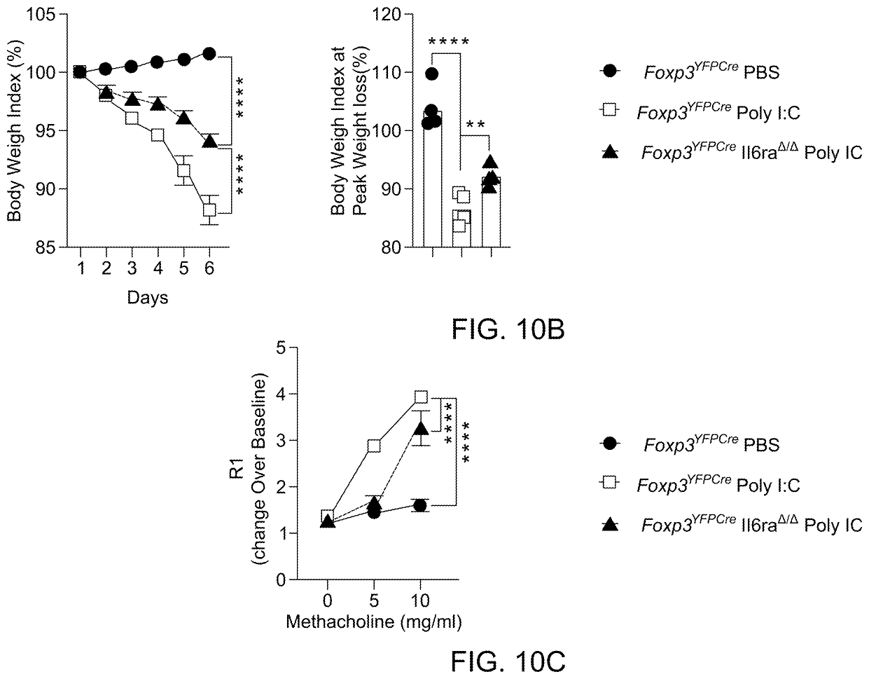

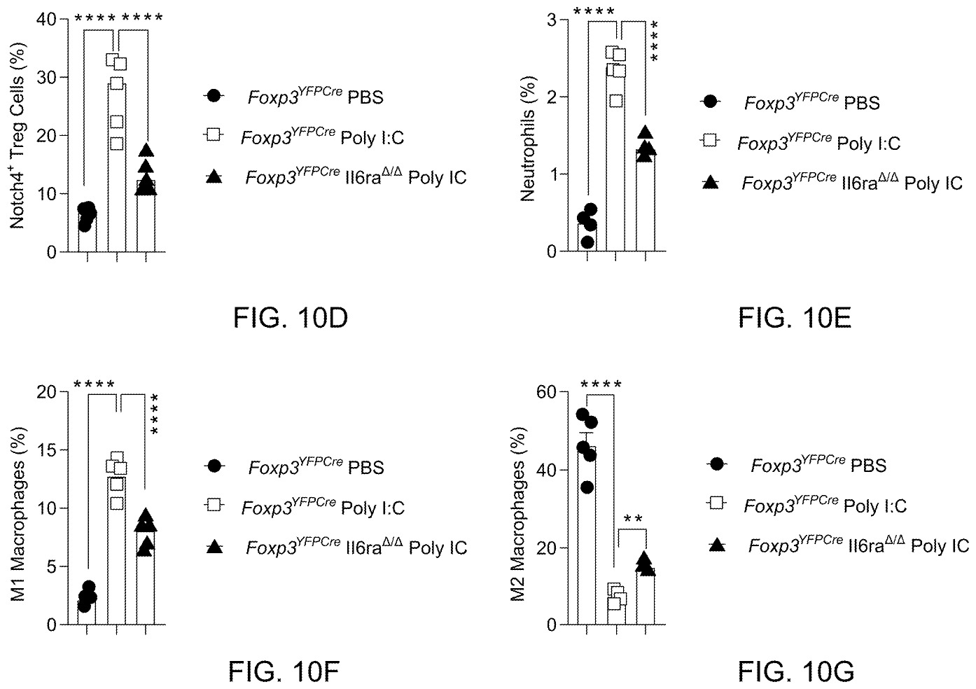

Treg cell Notch4 promotes lung tissue inflammation induced by polyinosinic: polycytidylic acid (Poly I:C). To investigate the mechanism by which Notch4 licenses lung tissue inflammation in viral respiratory infections, we first examined its role in Poly I:C-induced airway inflammation. Poly I:C stimulates Toll like receptor 3 and the downstream viral RNA sensors cytoplasmic retinoic acid—inducible gene I (RIG-I) and melanoma differentiation-associated protein 5 (MDAS), thus providing a proxy model of RNA viral infections (Broggi et al., 2020; Iwasaki and Pillai, 2014; Kato et al., 2006). Daily intra-tracheal instillation of Poly I:C in mice for 6 consecutive days resulted in a sharp progressive increase in the frequencies and absolute numbers of Notch4 expressing lung tissue Treg cells as well as the MFI of Notch4 on those cells (FIG. 2A-2C). In contrast, minimal expression of Notch4 was noted on CD4+ Teff cells. Treg cell specific deletion of Notch4 using a Foxp3-driven Cre recombinase (Foxp3YFPCre) and floxed Notch4 allele (Foxp3YFPCreNotch4Δ/Δ) or treatment with an anti-Notch4 neutralizing monoclonal antibody (mAb) but not an isotype control mAb protected the mice from the weight loss induced by the Poly I:C treatment (FIG. 2D, 2E). Histopathological analysis revealed intense inflammation in the lungs of the Poly I:C-treated mice that was suppressed by Notch4 antagonism (FIG. 2F, 2G). Similarly, airway hyperresponsiveness (AHR), which was increased in Poly I:C-treated mice, was also normalized (FIG. 2H). Analysis of bronchoalveolar lavage (BAL) fluid revealed increased IL-6 concentrations in the Poly I:C-treated mice that were suppressed upon Notch4 inhibition, consistent with decreased inflammation (FIG. 2I). A similar increase was also noted for a number of other cytokines including IFNγ, IL-1α, IL-1β, TNFα, IFNβ, MCP1, GM-CSF, IL-17, oncostatin M, leukemia inhibitory factor and CXCL5 most of which were also suppressed by Notch4 antagonism (FIG. 10A). Further analysis revealed that Notch4 inhibition suppressed lung tissue neutrophil infiltration induced by Poly I:C treatment and reversed the increase in lung M1 macrophages while increasing the M2 macrophages (FIG. 2J). In vitro co-cultures of Poly I:C-treated macrophages with lung Treg cells derived from Poly I:C-treated mice revealed that Foxp3YFPCreNotch4Δ/Δ but not Foxp3YFPCre Treg cells reversed the M1 skewing and promoted M2 macrophage differentiation (FIG. 2K). These results confirmed a critical role for Treg cell Notch4 expression in the evolution of lung inflammation induced by Poly I:C.

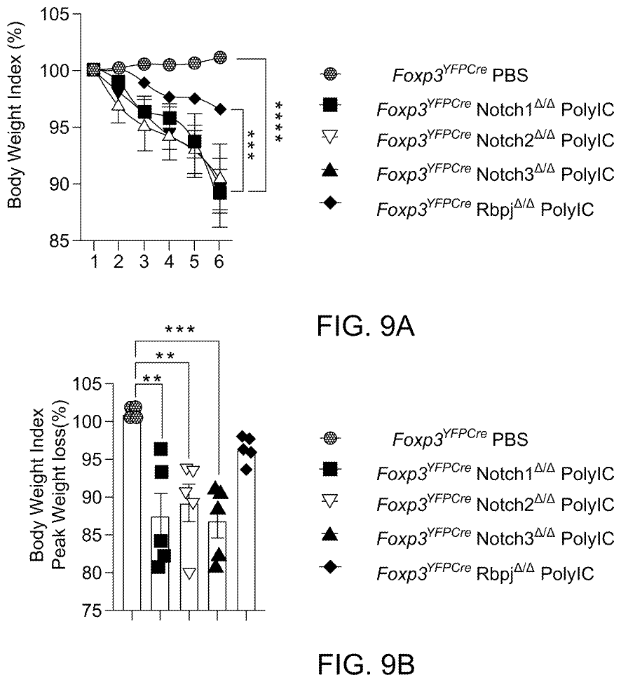

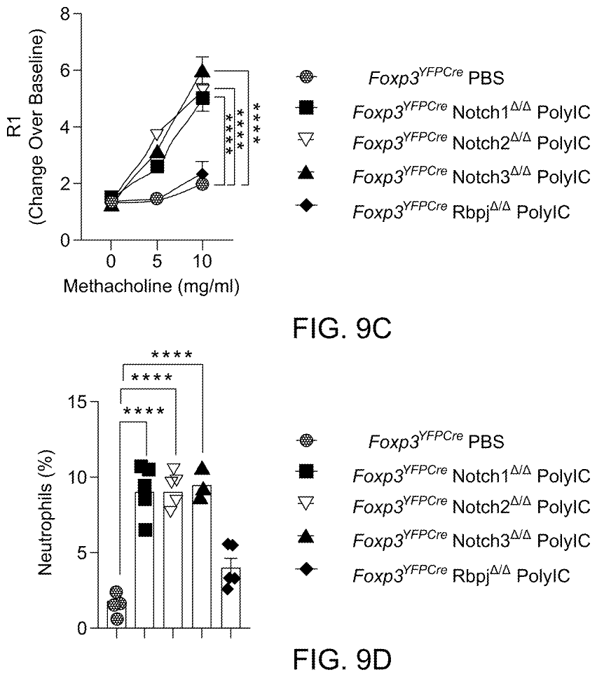

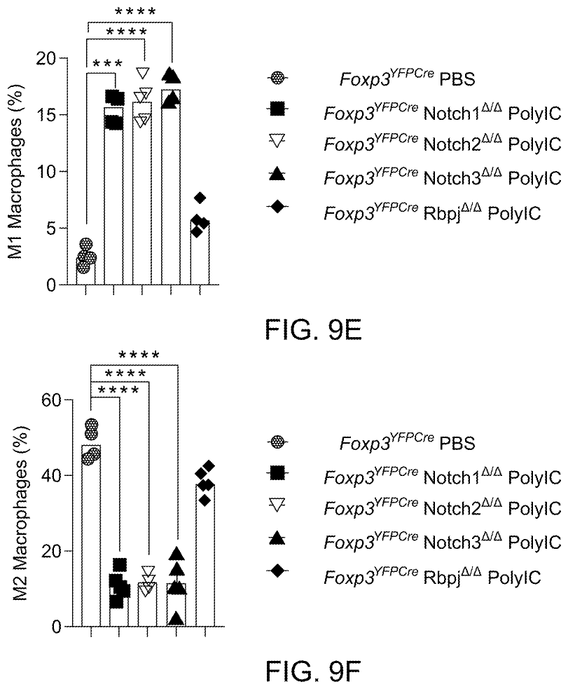

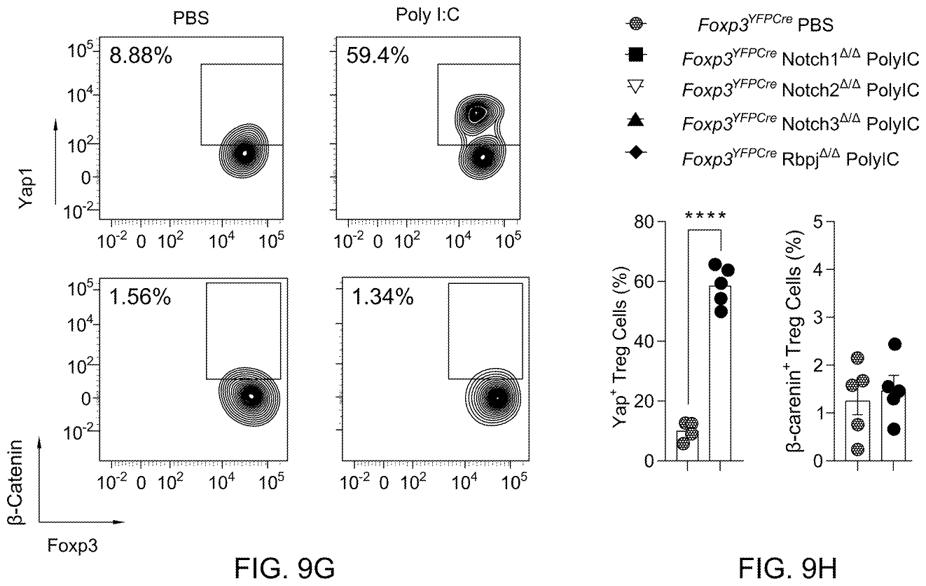

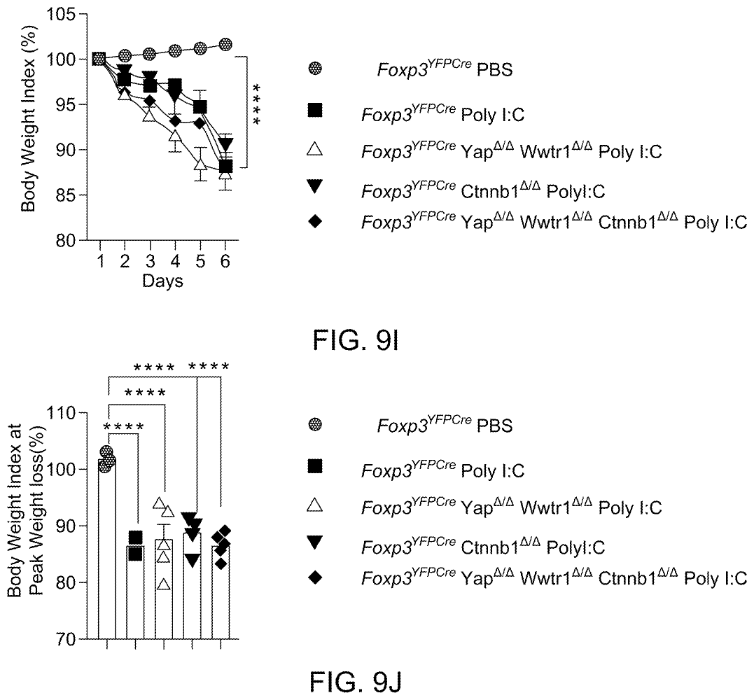

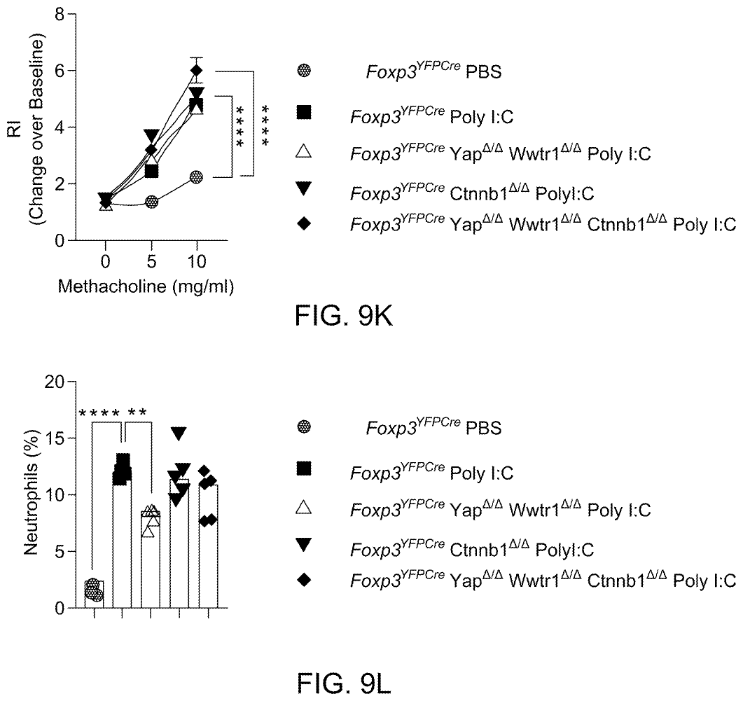

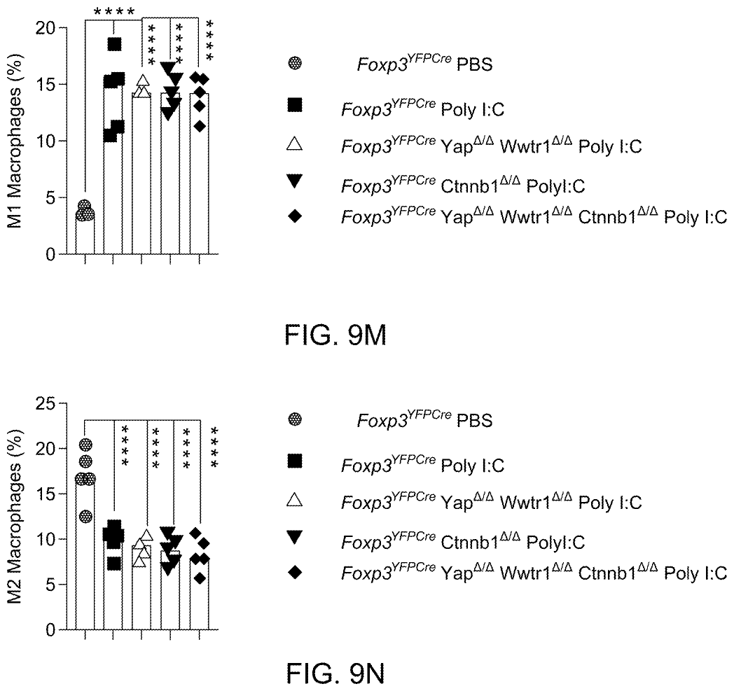

Different Notch receptors control Treg cell function (Charbonnier et al., 2015; Ostroukhova et al., 2006; Perumalsamy et al., 2012). However, the role of Notch4 in licensing lung tissue inflammation by Poly I:C was highly specific in that Treg cell-specific deletion of the other Notch genes, including Notch1, Notch2 and Notch3, failed to protect against tissue inflammation. In contrast, Treg cell-specific deletion of Rbpj, encoding the Notch canonical pathway transcription factor RBPJ, largely reproduced the protective effect of Notch4 deletion (FIG. 9A-9F). Downstream of Notch4, our previous studies have implicated the Hippo and Wnt pathways in Treg cell regulation of Th17 and Th2 responses in allergic airway inflammation, respectively (Harb et al., 2020). Poly I:C treatment potently induced the expression of the Hippo pathway effector Yap in lung tissue Treg cells, while the expression of Wnt pathway effector β-Catenin was unchanged (FIG. 9G, 9H). Treg cell-specific deletion of Yap1 and Wwtr1, encoding the Hippo pathway effectors Yap and Taz, Ctnnb1, encoding the Wnt pathway effector β-Catenin, or all three genes failed to protect against Poly I:C-induced inflammation. These findings indicated that Notch4 regulation of the acute inflammatory responses to viral infection proceeds by distinct mechanisms (FIG. 9I-9N).

We have previously identified a key role for IL-6 in upregulating Notch4 expression on Treg cells in asthmatic inflammation (Harb et al., 2020). Consistent with these results, Notch4+ lung Treg cells from Poly I:C-treated mice expressed higher levels of IL-6Rα chain as compared to their Notch4− counterparts (FIG. 2L). To further elucidate the mechanisms of Notch4 induction in Treg cells, we conducted in vitro studies to examine the capacity of Poly I:C and cytokine treatment to upregulate Notch4 expression in Treg cells isolated from PBS or Poly I:C-treated mice. Results showed that Poly I:C treatment of lung but not splenic Treg cells increased Notch4 expression in synergy with IL-6 but not IL-33 (FIG. 2M). In vivo, Treg cell-specific deletion of Il6ra attenuated the Poly I:C-induced Notch4 expression on lung Treg cells and the attendant tissue inflammatory response, including the decline in body weight, the increase in AHR, the influx of neutrophils and the alteration in macrophage populations (FIG. 10B-10G). These results indicated that both IL-6 and additional signals contribute to Notch4 upregulation on lung Treg cells in Poly I:C-treated mice.

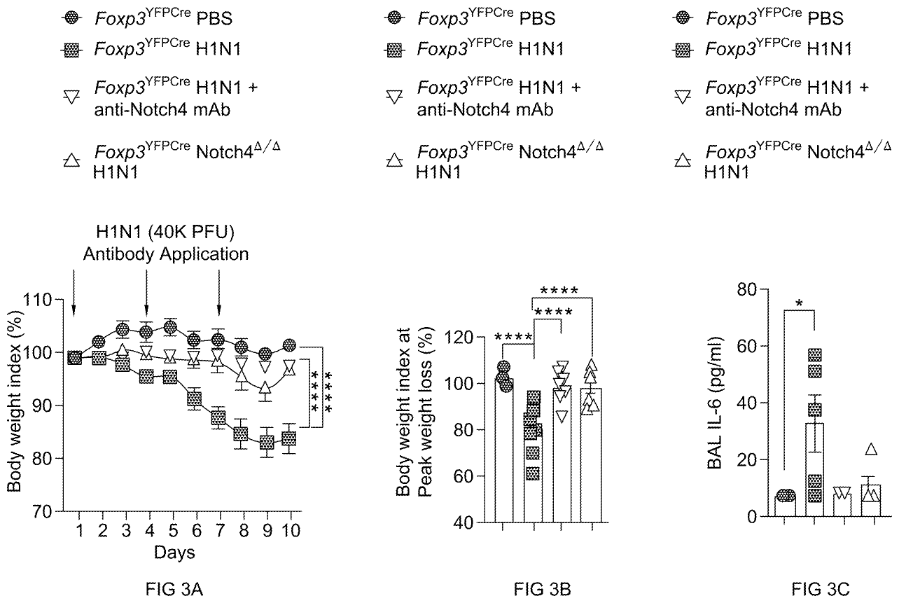

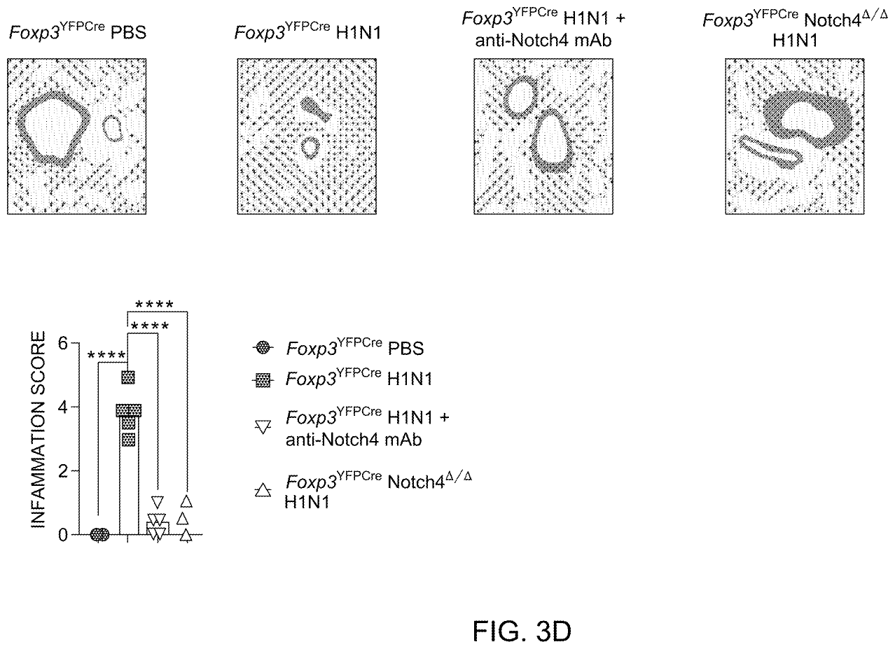

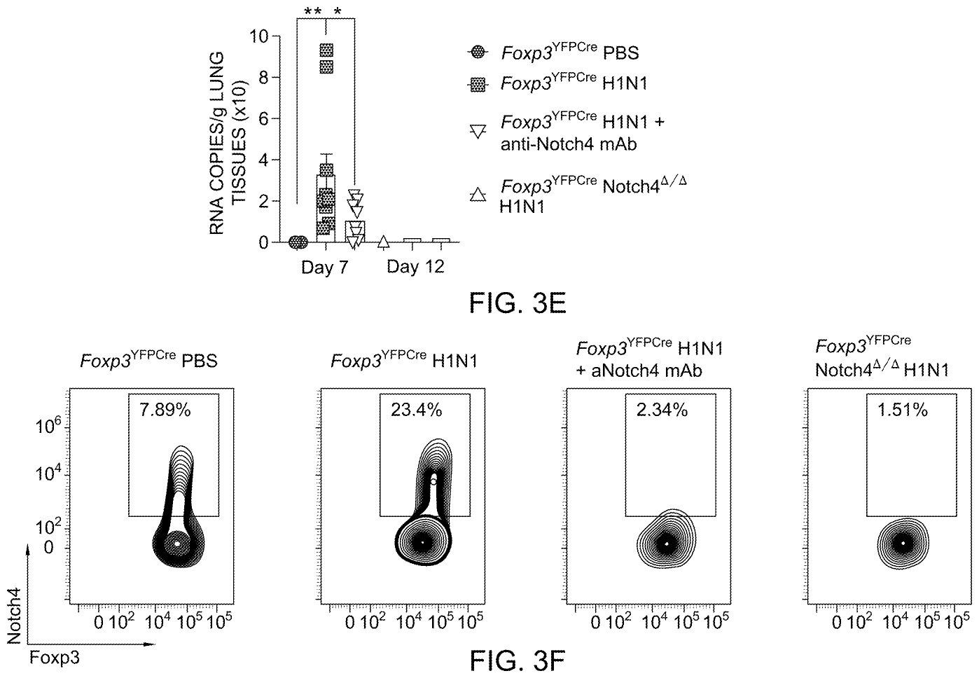

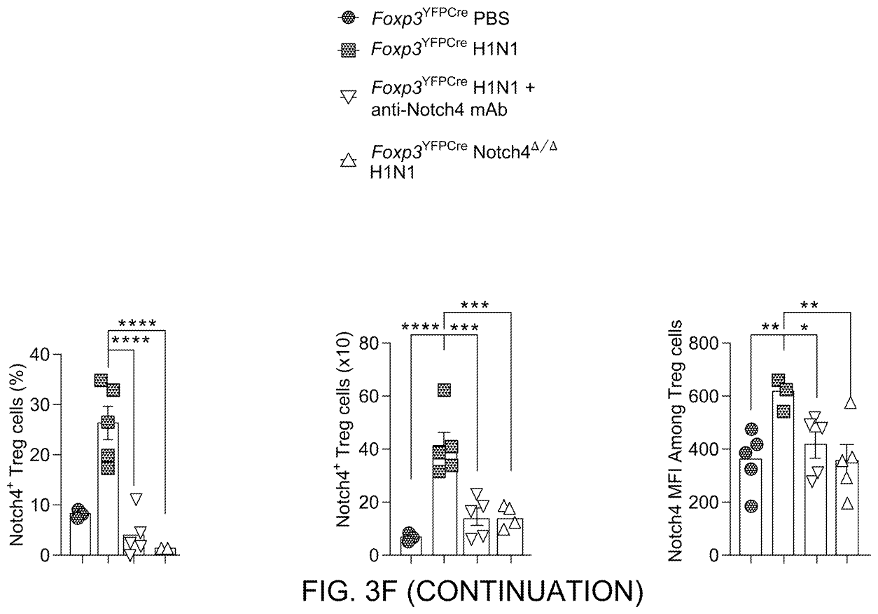

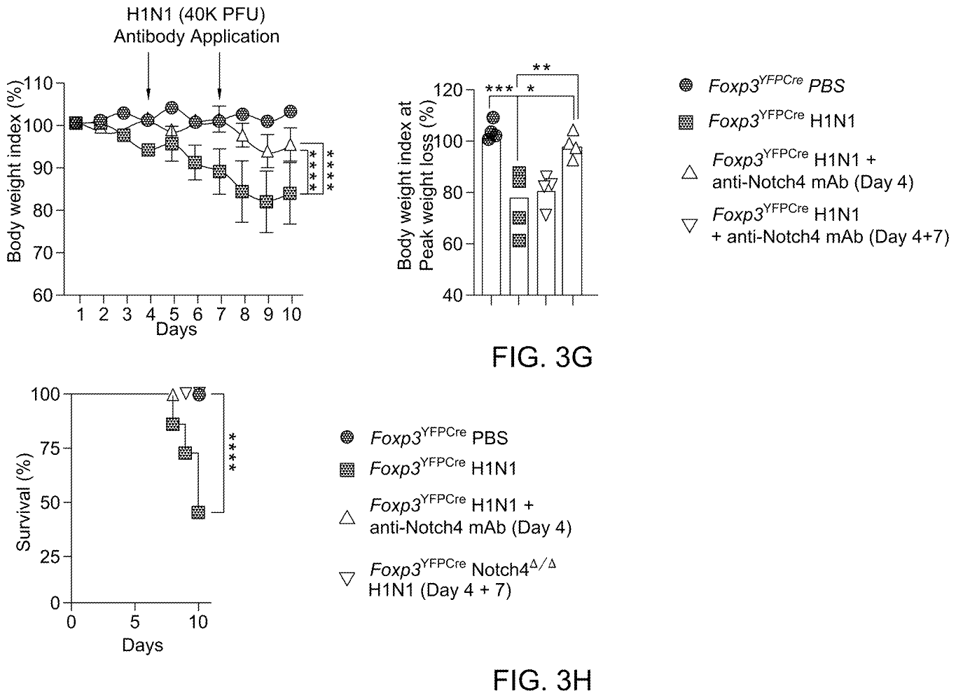

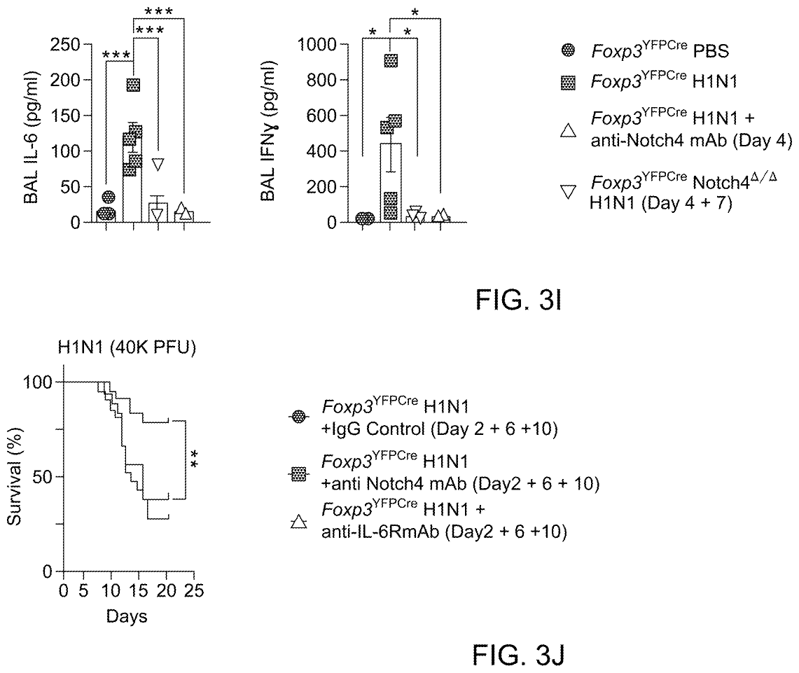

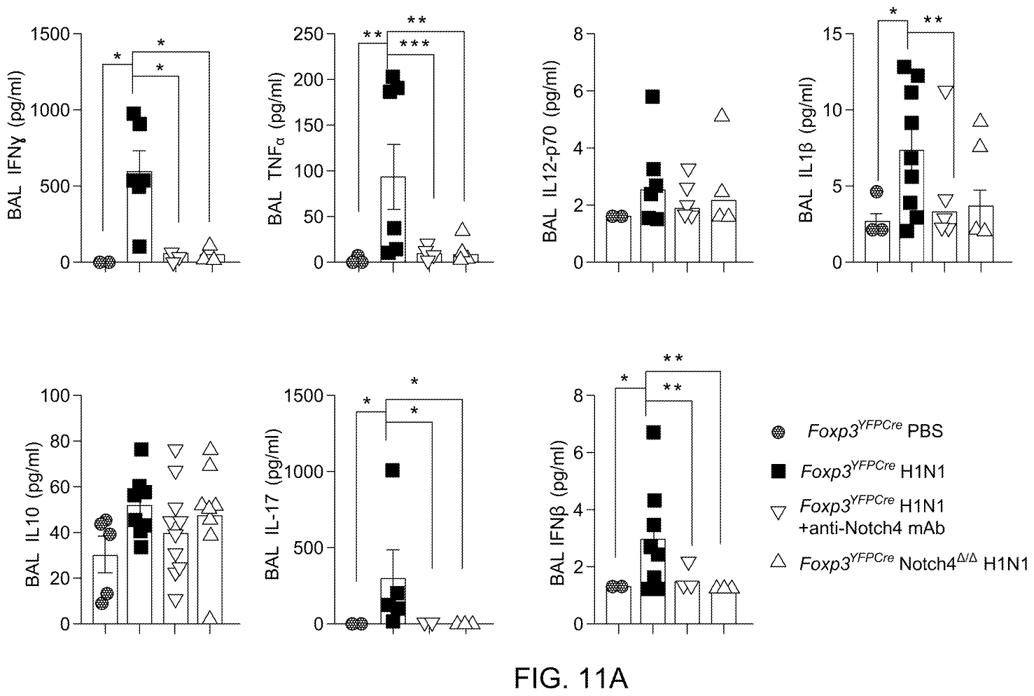

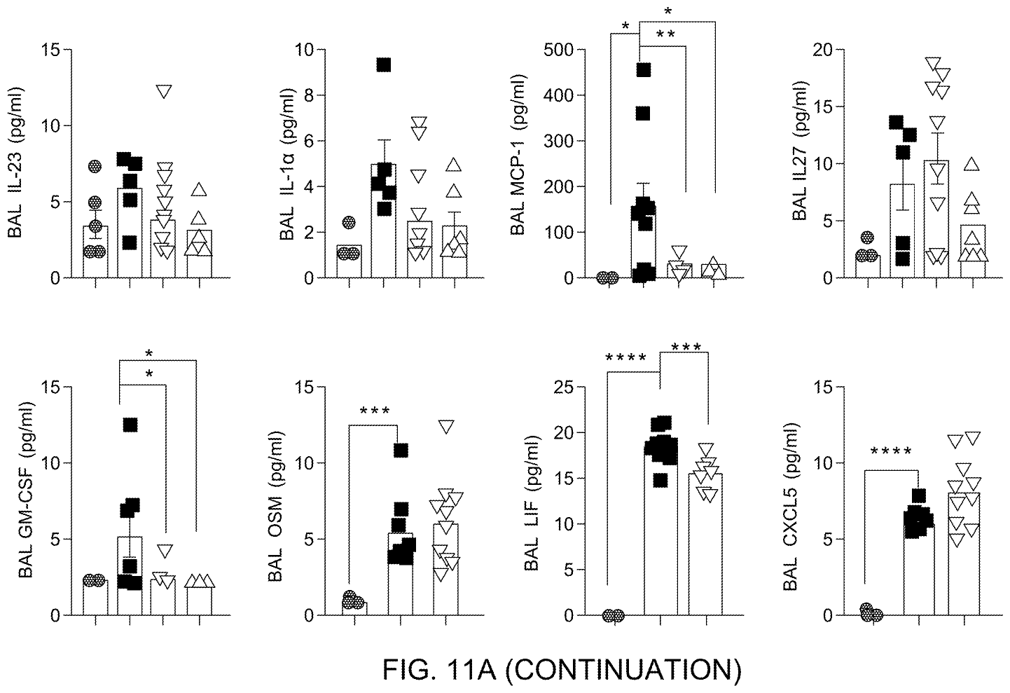

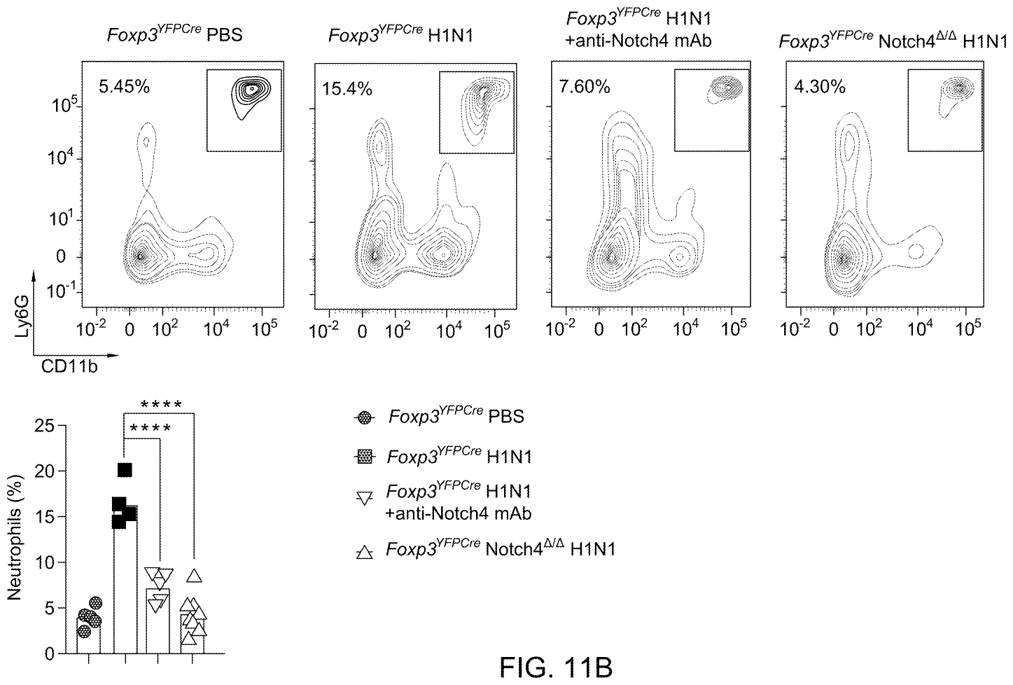

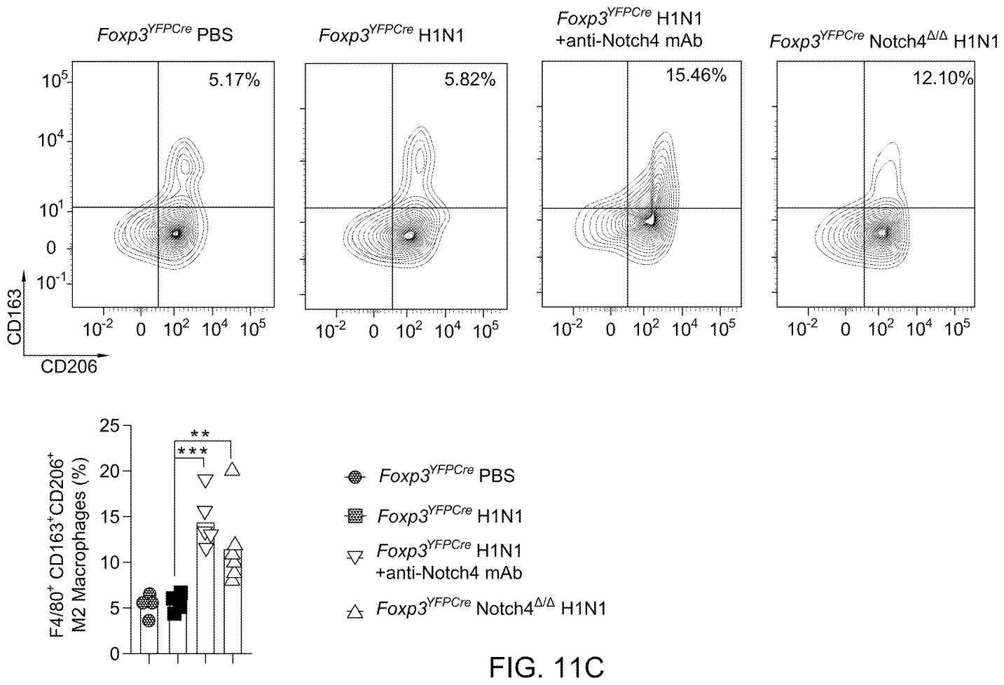

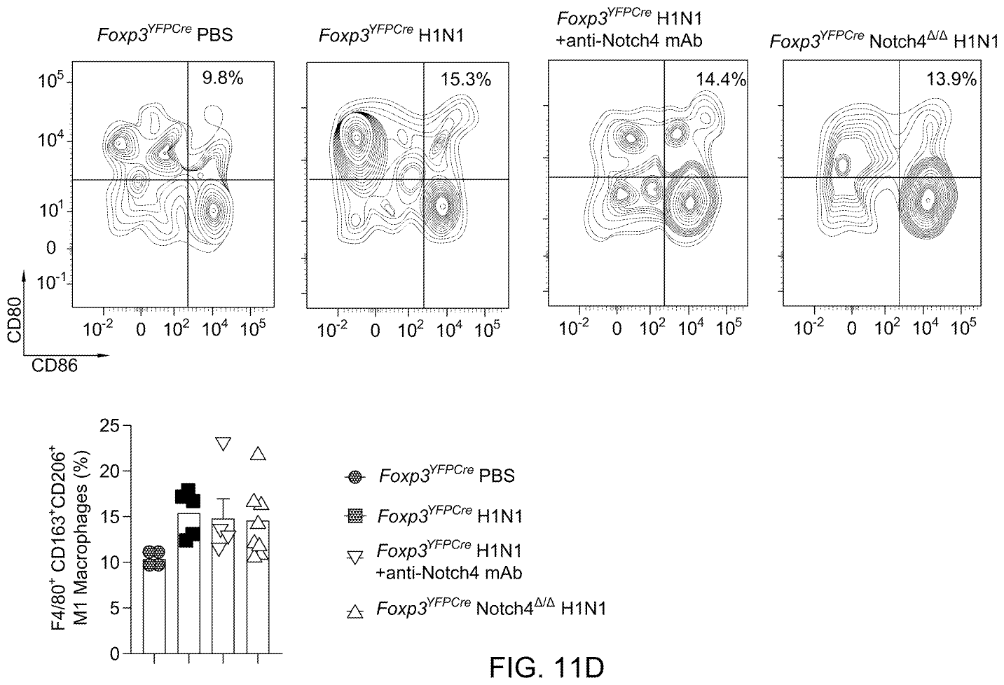

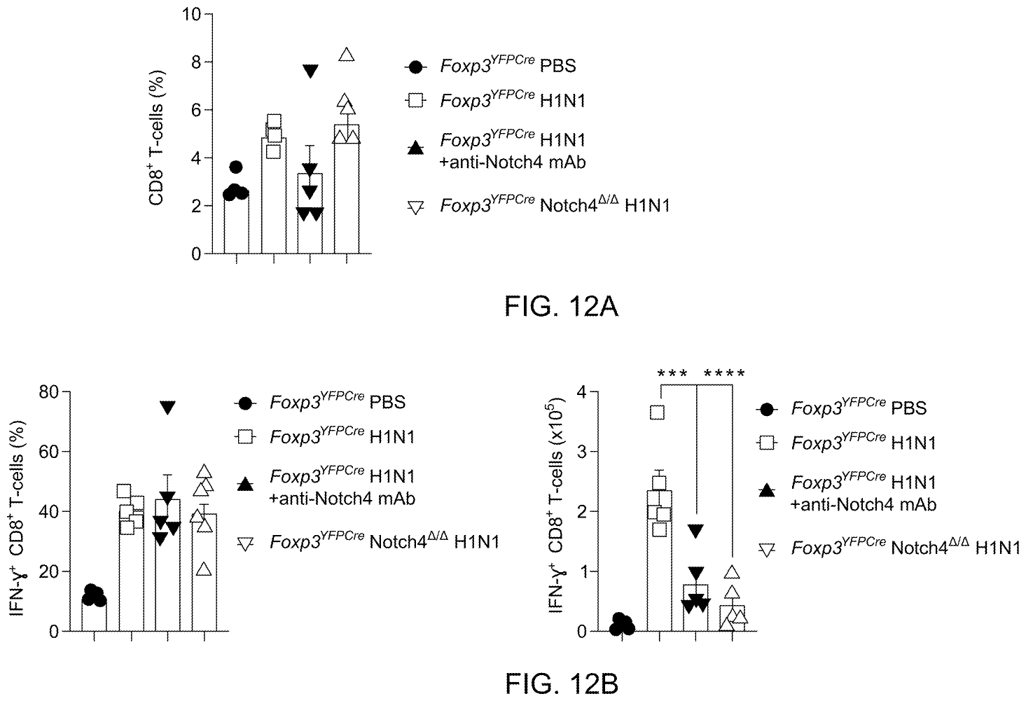

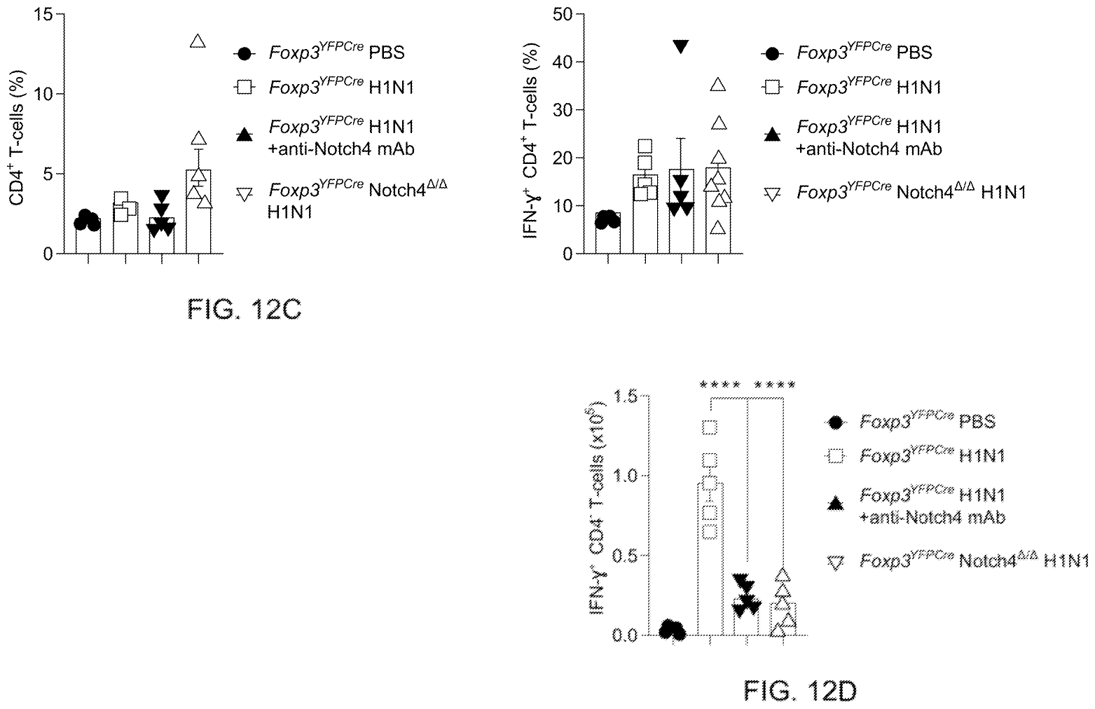

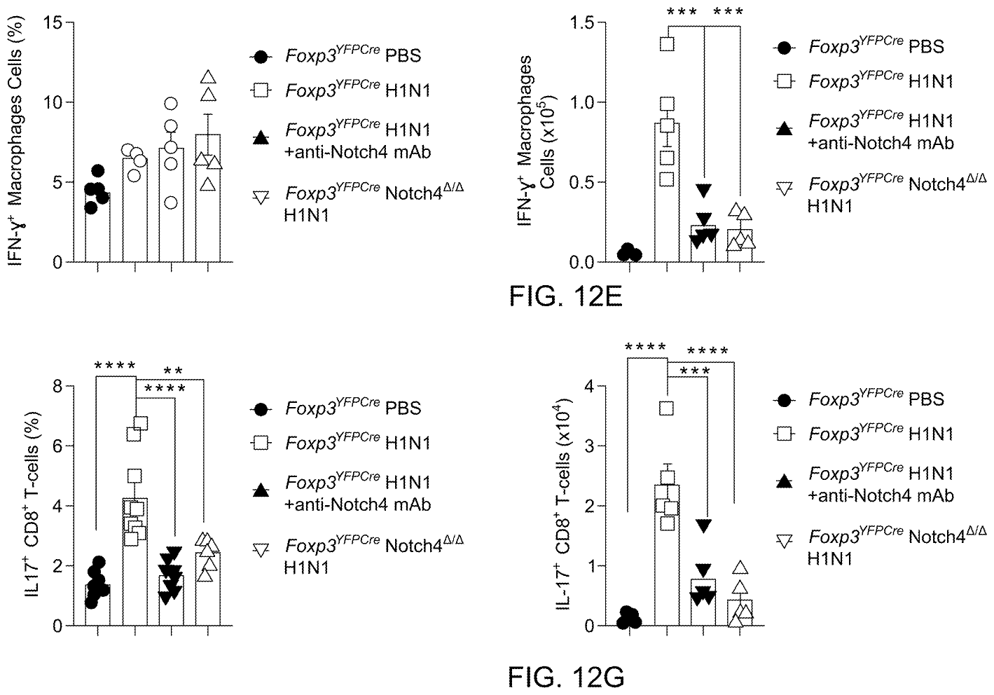

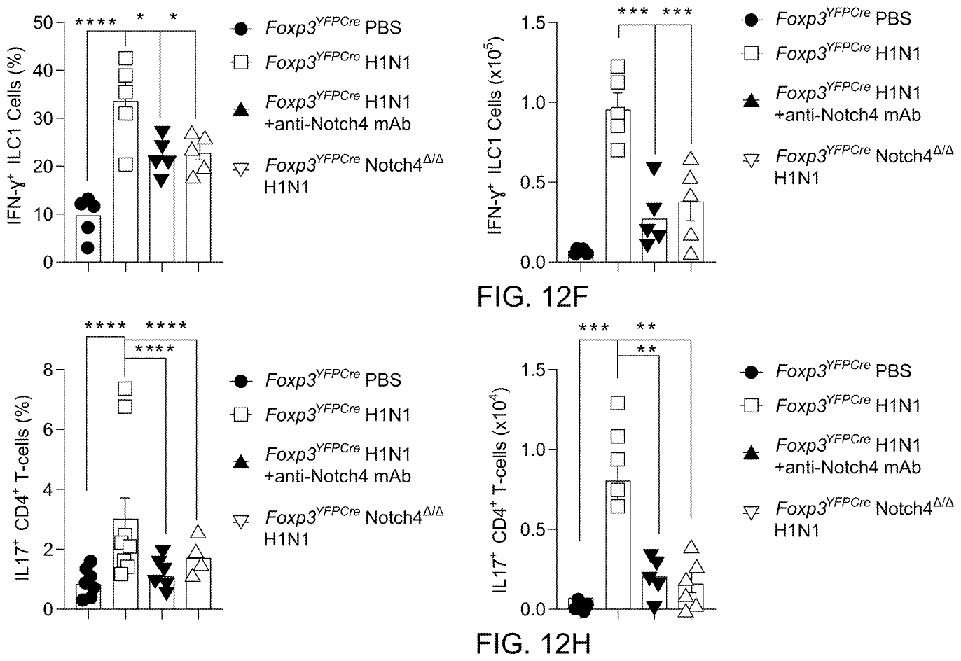

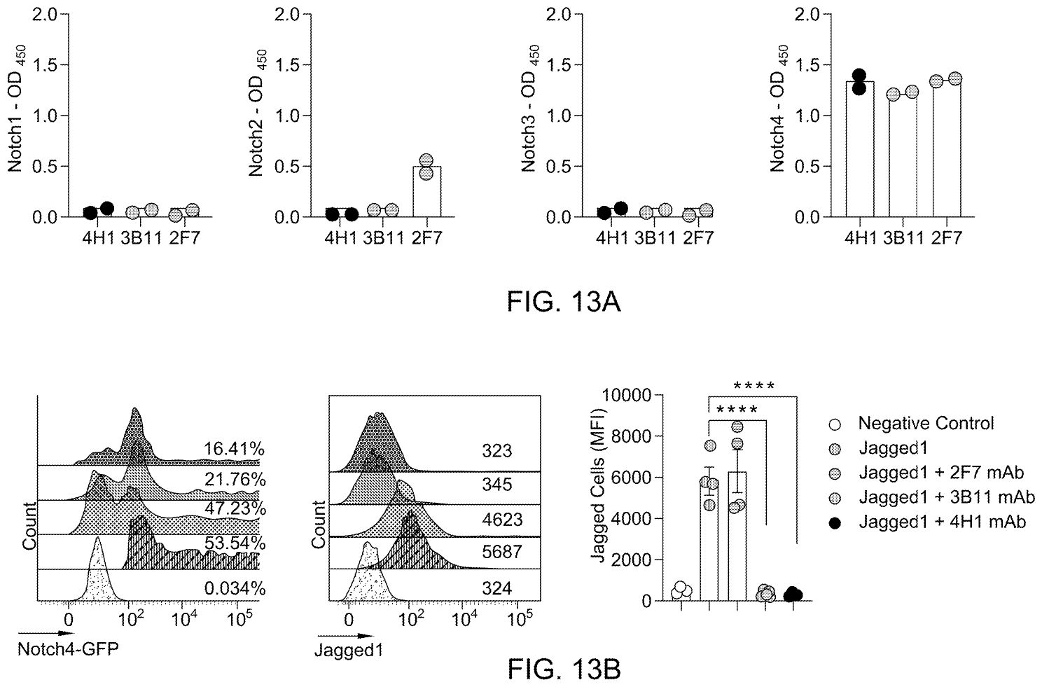

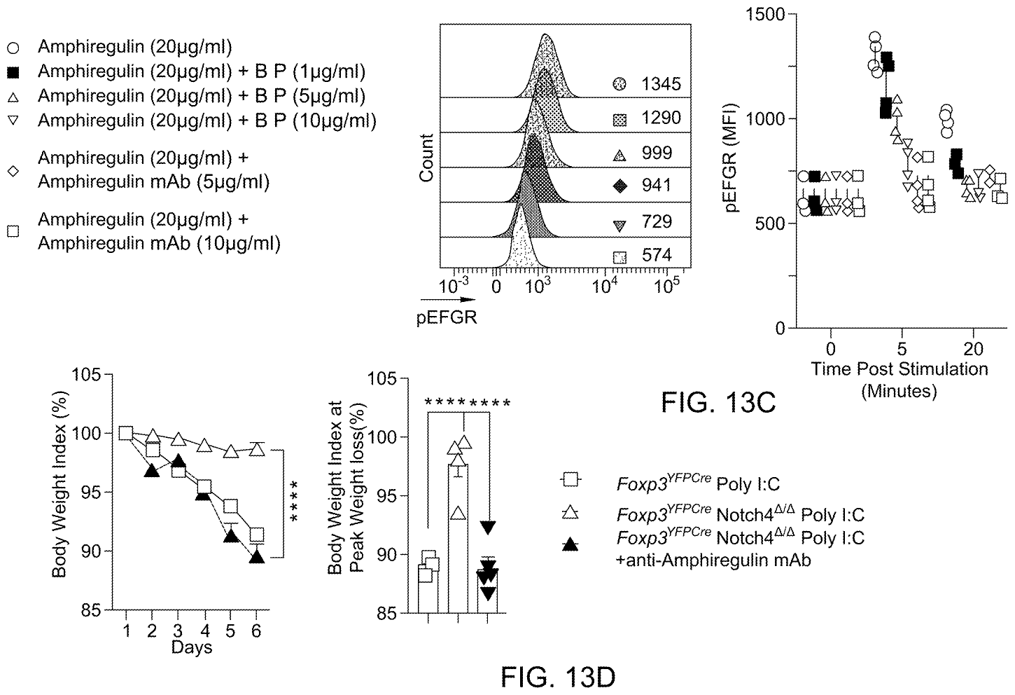

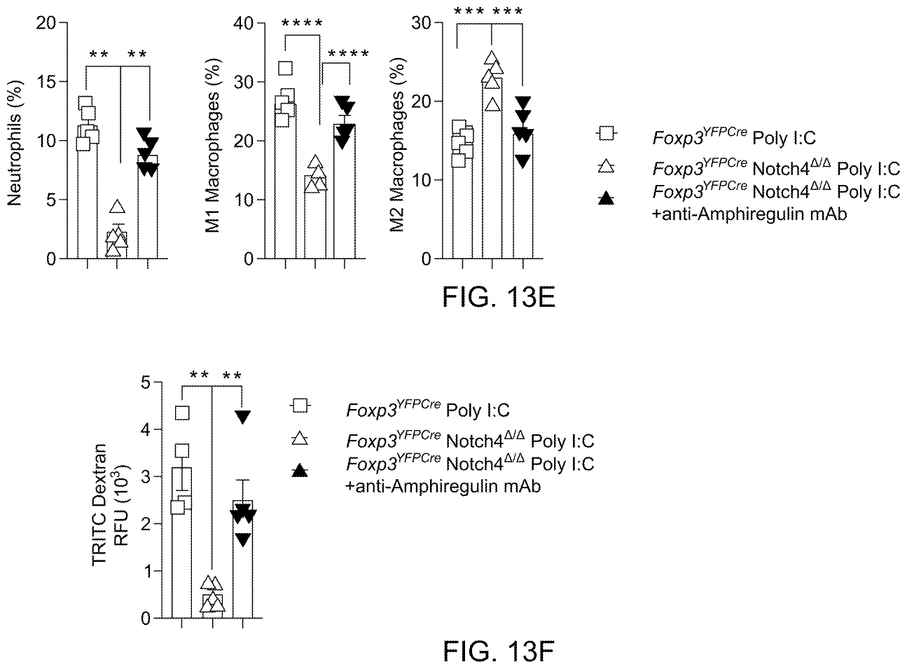

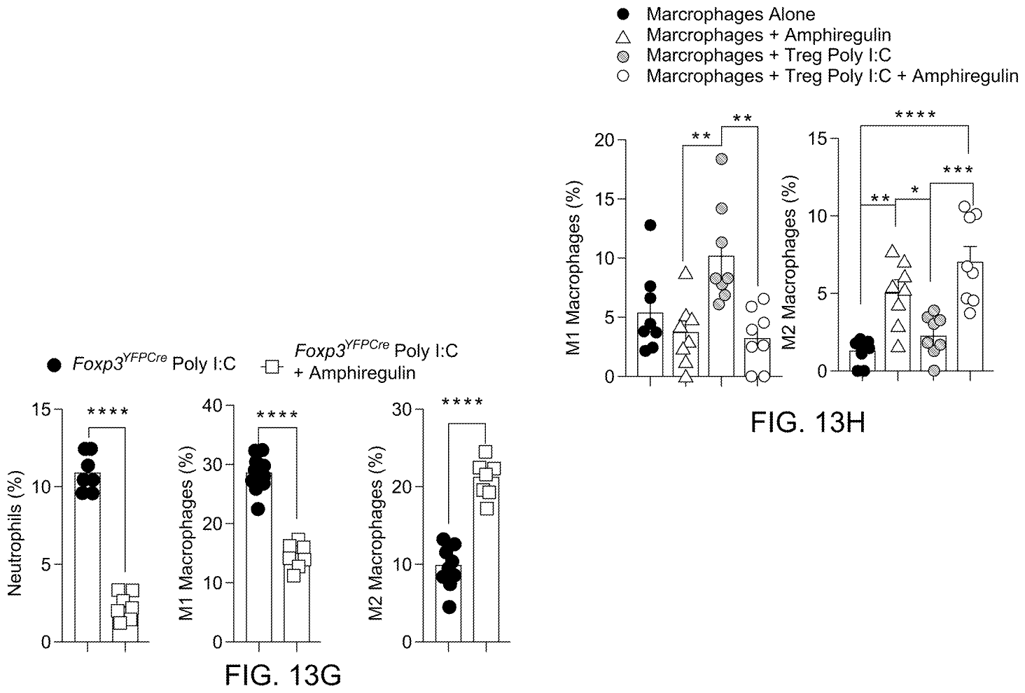

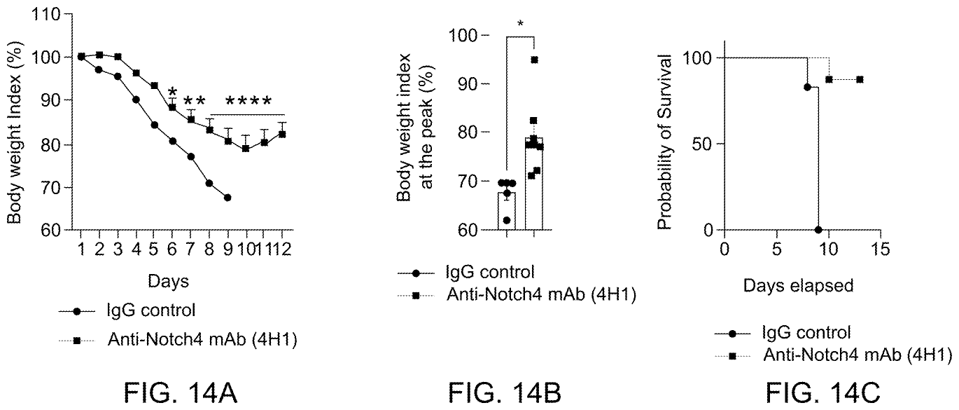

Notch4 promotes lung inflammation induced by H1N1 Influenza A virus. To extend these findings into an RNA viral model, we analyzed the role of Notch4 expression on Treg cells in licensing lung tissue inflammation in a mouse model of H1N1 Influenza A virus infection (Woodham et al., 2020). In studies in which mice were infected with H1N1 virus at 4×104 plaque forming units (pfu)/mouse, the weight loss induced by the infection was abrogated by prior treatment with a neutralizing anti-Notch4 mAb or upon Treg cell-specific deletion of Notch4 (FIG. 3A, 3B). Notch4 antagonism suppressed the virus-induced increase in BAL fluid IL-6 concentrations (FIG. 3C). It also suppressed the induction by the viral infection of a number of other inflammatory cytokines, similar to what was noted for the Poly I:C treatment (FIG. 11A). It also reversed the lung tissue inflammatory response in the infected mice (FIG. 3D). Antagonism of Notch4 expression did not result in increased viral loads as measured on days 7 and 12 post infection (FIG. 3E). Analysis of Notch4 expression on lung Treg cells in the respective mouse groups revealed that H1N1 infection increased Notch4 expression, an effect abrogated by treatment with an anti-Notch4 mAb or by Treg cell-specific Notch4 deletion (FIG. 3F). Both interventions greatly attenuated lung tissue neutrophil influx, restored the alveolar macrophage population and reversed the skewing of tissue macrophages away from the pro-inflammatory M1 phenotype towards an anti-inflammatory M2 phenotype (FIG. 11B-11D). Notch4 inhibition did not impact the frequencies of IFN′y+CD4+ and IFN′y+CD8+ T cells, and IFN′y+ macrophages infiltrating the lungs of infected mice, while those of IFN′y+ innate lymphoid cells 1 (ILC1) were decreased. Nevertheless, the absolute numbers of all four populations were sharply decreased, consistent with the markedly attenuated inflammatory response (FIG. 12A-12F). Notch4 inhibition also decreased the frequencies and the absolute numbers of lung tissue IL-17+CD4 and CD8 T cells (FIG. 13G, 13H).

To determine the capacity of Notch4 antagonism to rescue active disease, mice were infected with the same dose of H1N1 virus and were either treated with an isotype control mAb or with a Notch4 mAb following the onset of infection. Results revealed that treatment of mice with Notch4 mAb at days 4 or at days 4 and 7 post infection rescued mice from death, whereas only days 4 and 7 treatment suppressed the weight loss and innate immune activation associated with the infection (FIG. 3G, 3H). Both Notch4 mAb treatment regiments suppressed the increase BAL fluid IL-6 and IFN′y concentrations induced by the viral infection (FIG. 3I). We further compared different Notch4 mAb treatment regimens for their capacity to rescue mice from a lethal dose 75% (LD75) of H1N1 virus (at 7×104 pfu/mouse). Treatments with the Notch4 mAb on days 2, 6 and 10 was associated with decreased mortality (22%). In contrast, treatment with an isotype control mAb or with an anti-IL-6Rct mAb failed to protect the mice from exacerbated mortality (62% and 72% mortality, respectively) (FIG. 3J). Collectively, these results indicated that therapy with an anti-Notch4 mAb protects mice with active influenza virus infection.

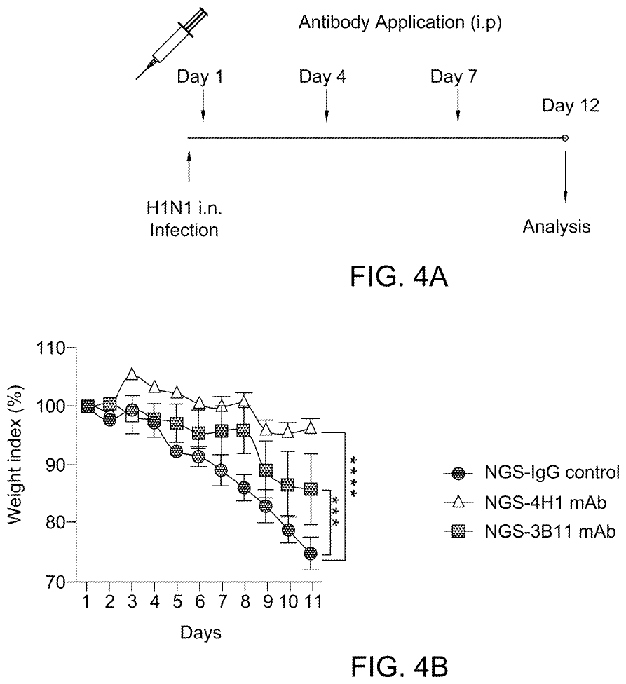

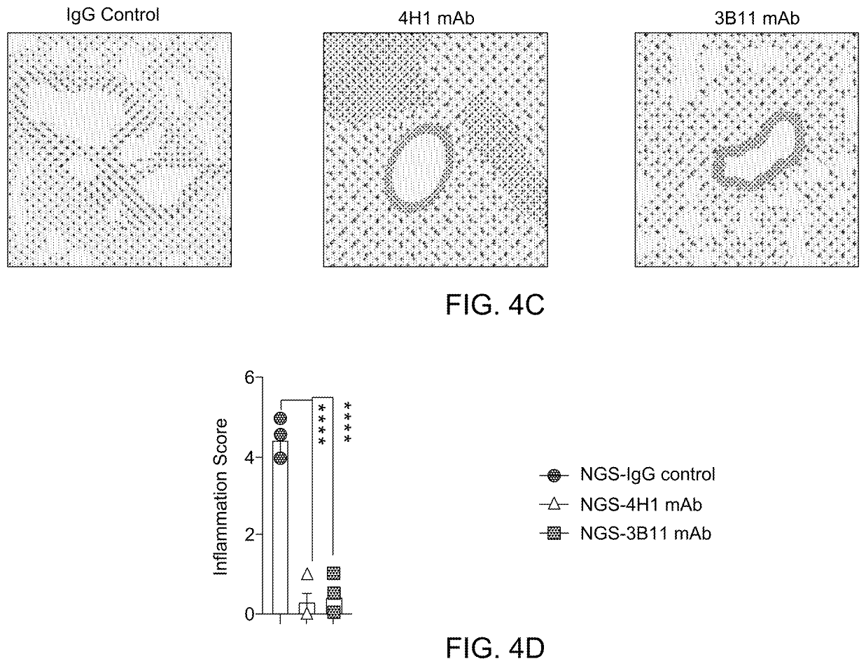

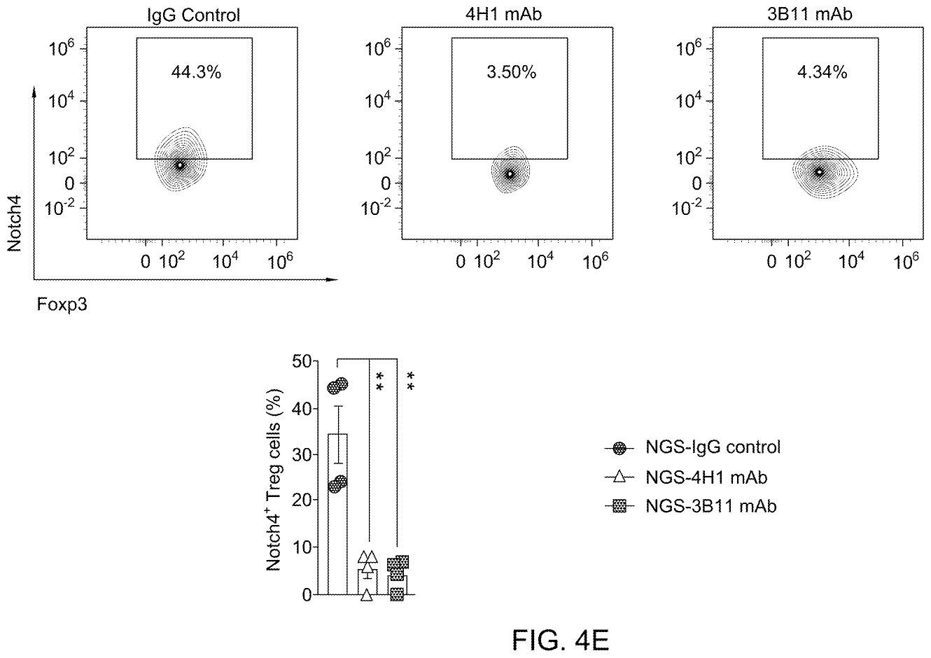

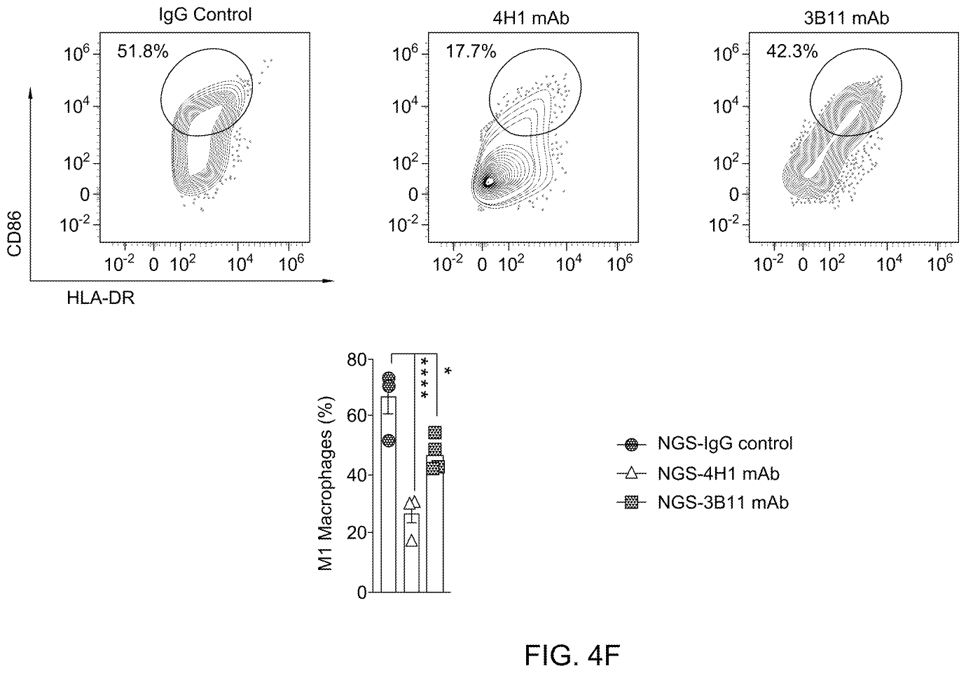

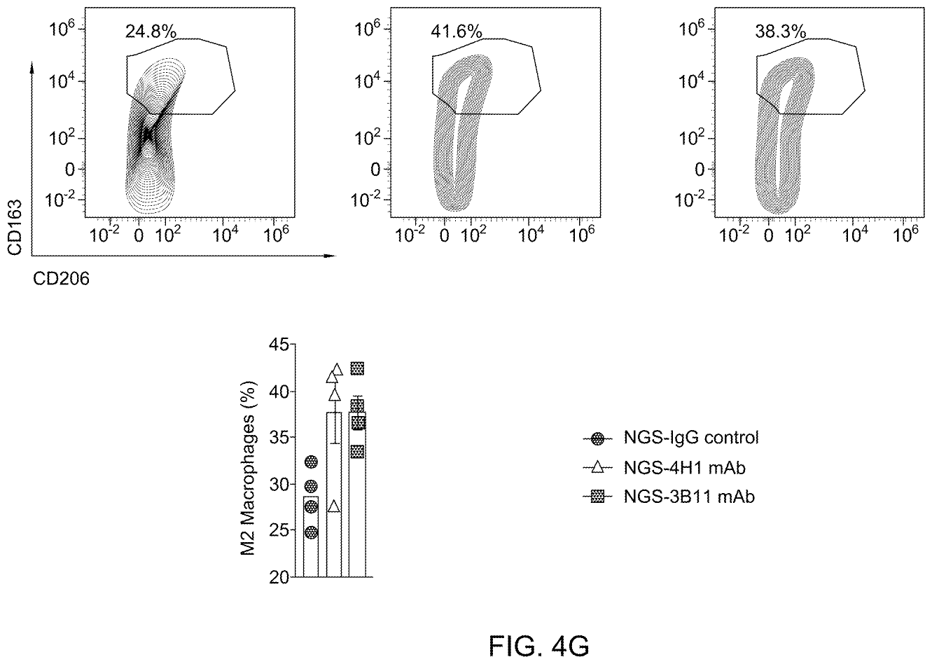

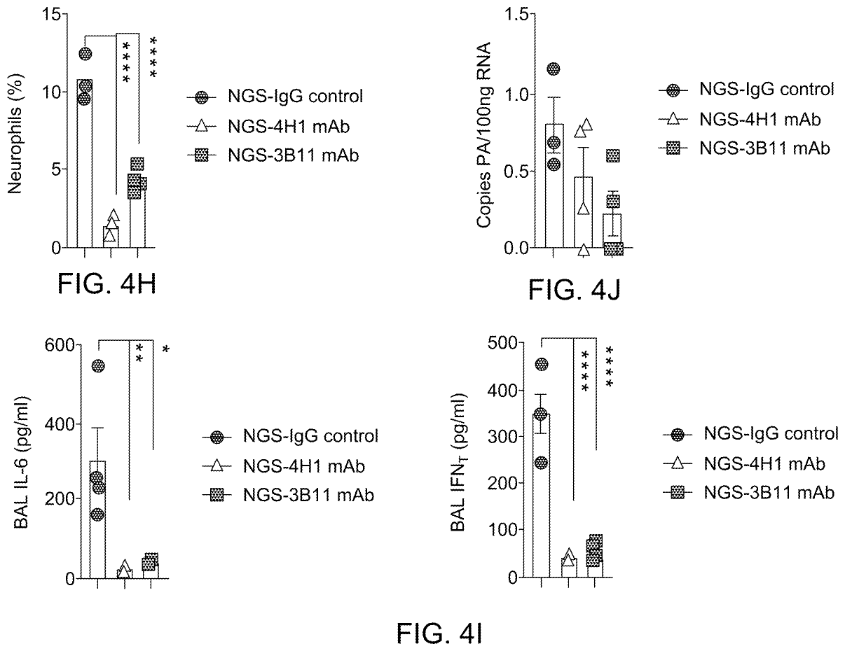

To extend the above results to the setting of a humanized immune system mouse model, we analyzed the capacity of neutralizing anti-human Notch4 mAbs to rescue disease in NOD-PrkdcscidIl2rgtmiwjl/Sz (NSG) (NSG) mice reconstituted with human PBMCs and infected with the H1N1 virus (Verma et al., 2017). Neutralizing mAbs specific for Notch4 but not other Notch proteins were derived by immunizing mice with a recombinant N-terminal human Notch4 protein fragment (amino acids 1-637) (FIG. 13A-13B). Treatment of mice with one neutralizing anti-human Notch4 mAb (clone 4H1), but not an isotype control mAb, protected against H1N1 virus infection-induced weight loss and tissue inflammation (FIG. 4A-4D), Another neutralizing antibody (clone 3B11) was partially protective. Both antibodies inhibited Notch4 expression on lung Treg cells (FIG. 4E), as well as inhibiting macrophage skewing, neutrophil infiltration and the production of the cytokines IL-6 and IFNγ (FIG. 4F-4I). The antibody treatment did not impact the viral clearance as measured by virus copy numbers in lung tissue (FIG. 4J). These results confirmed that Notch4 regulates the magnitude of the human inflammatory response to H1N1 infection and that it can be successfully targeted for therapeutic intervention.

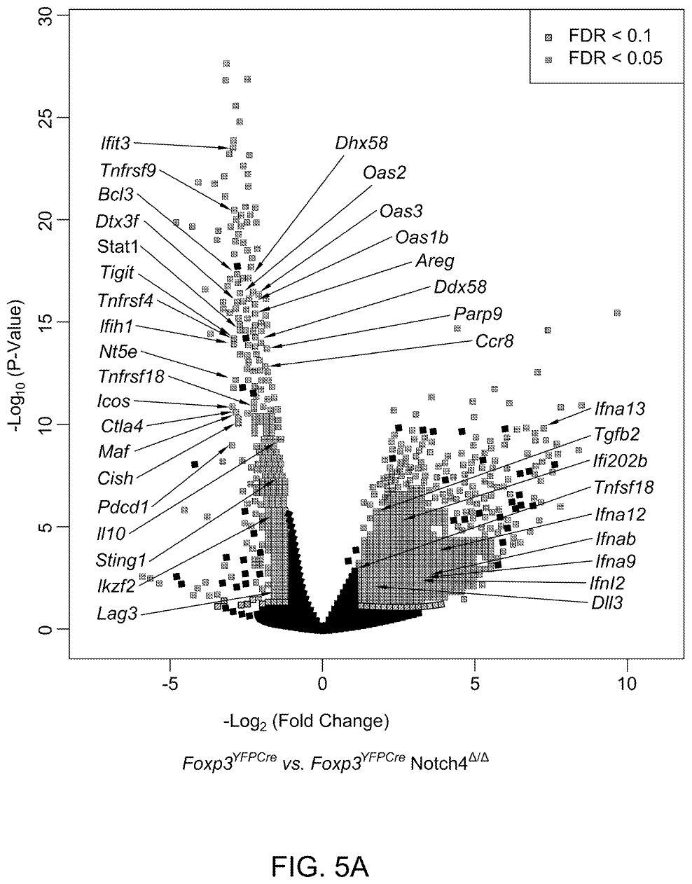

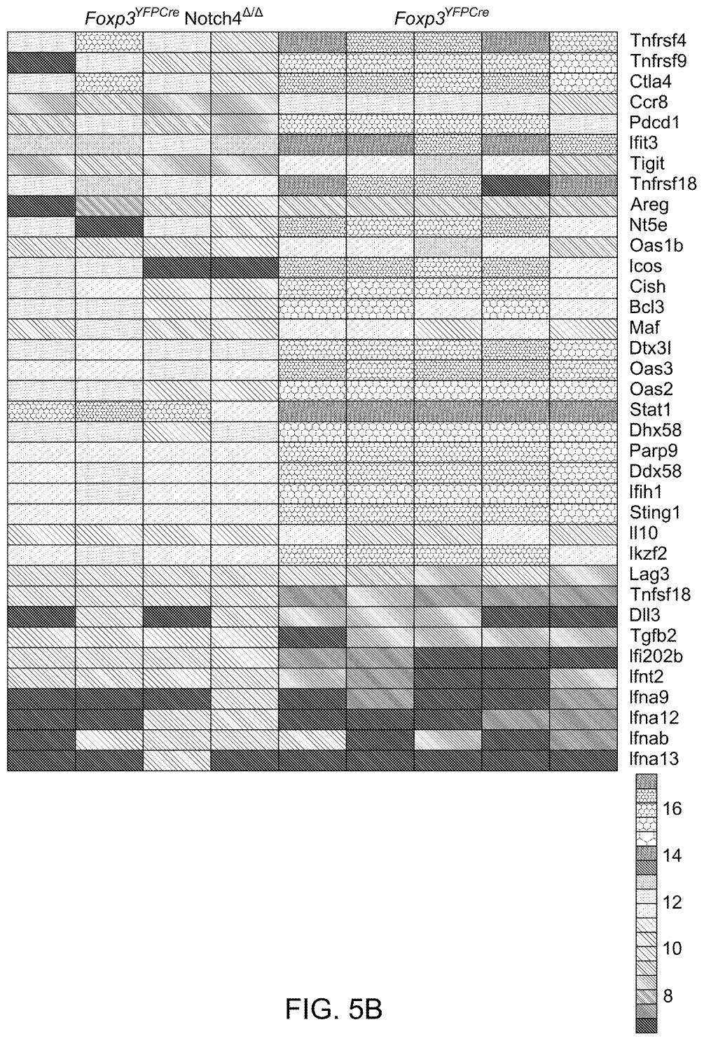

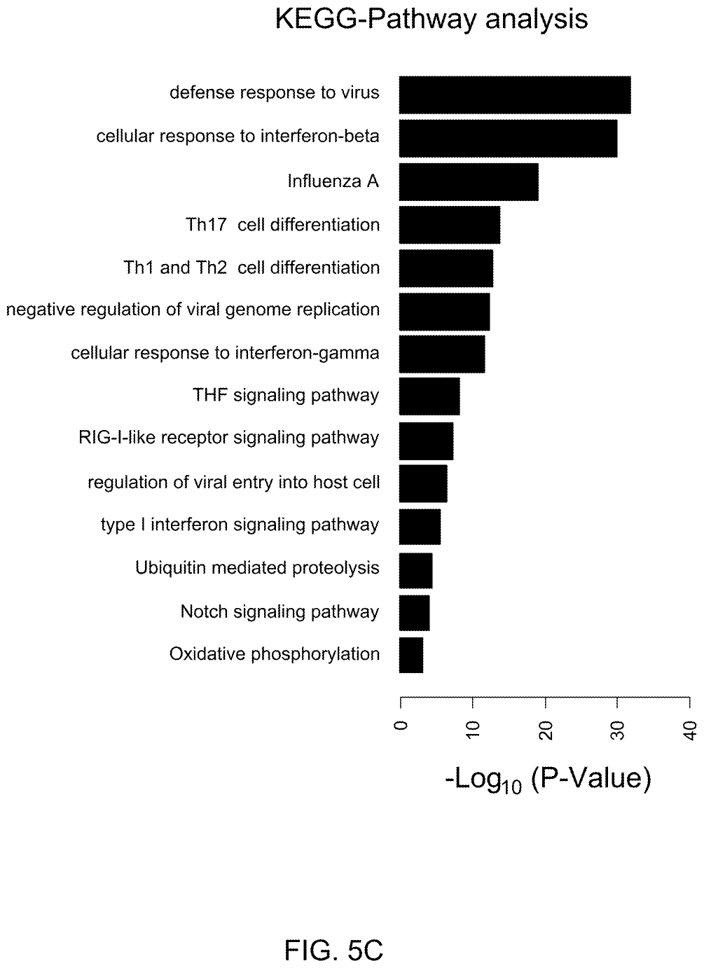

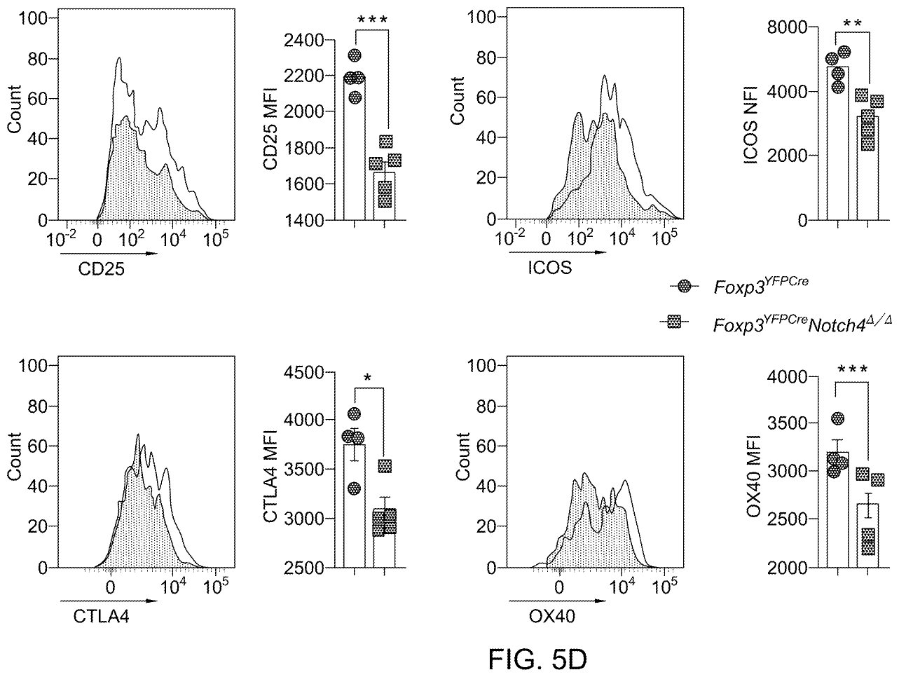

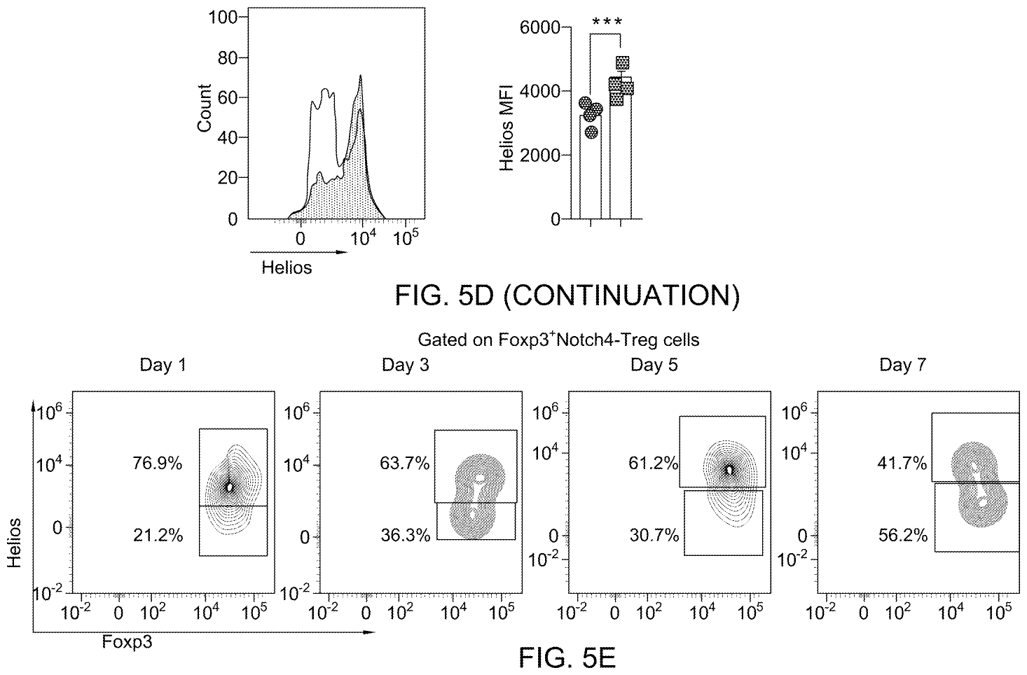

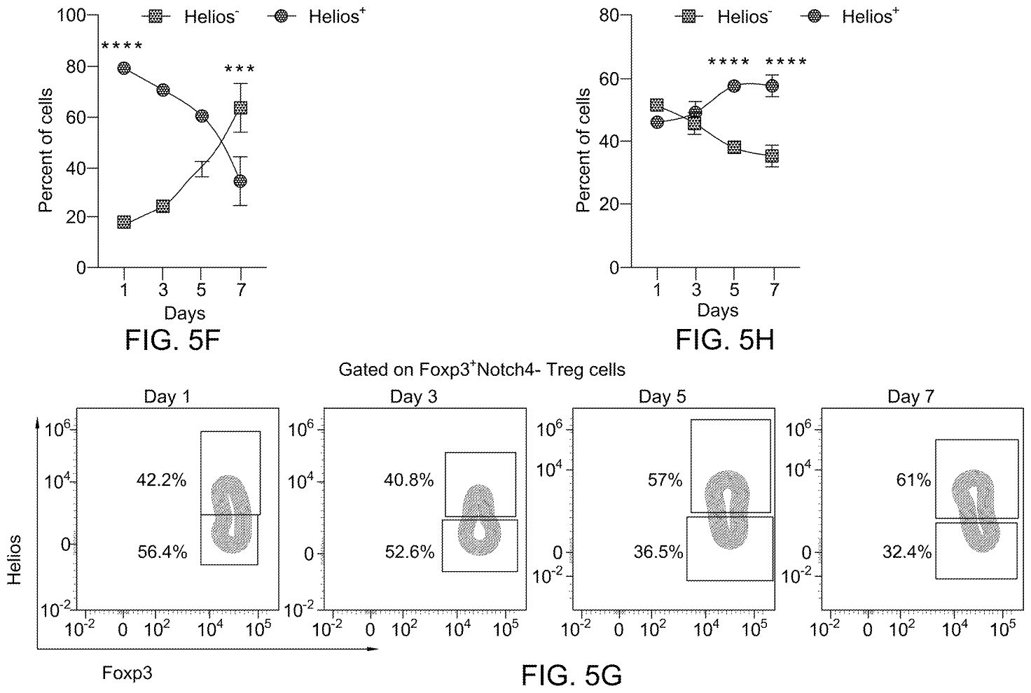

Protection by Notch4 antagonism is amphiregulin-dependent. To better understand the mechanisms by which Notch4 deficiency protected against lung inflammation, we analyzed the transcriptomes of lung Treg cells of Foxp3YFPCre and Foxp3YFPCreNotch4Δ/Δ mice treated with Poly I:C for 6 days. Results revealed that the lung Treg cells of Poly I:C treated Foxp3YFPCre mice exhibited an activated effector Treg cell signature, with upregulation of many canonical Treg cell transcripts including Ctla4, Ikzf2, Il10, Tigit and the TNF receptor superfamily members Tnfrsf4, Tnfrsf9 and Tnfrsf18 (Arpaia et al., 2015) (FIG. 5A, 5B). Poly I:C treatment also imparted an exhausted T cell-like signature with increased Pdcd1, Icos, Lag3 and other related transcripts that may impair their suppressive function (Lowther et al., 2016). In contrast, Treg cells of Poly I:C-treated Foxp3YFPCreNotch4Δ/Δ mice increased several type I interferon genes. More broadly, key pathways enriched in Foxp3YFPCre versus Foxp3YFPCreNotch4Δ/Δ lung Treg cells included those involved in innate viral sensing and response, type I interferon signaling, TH cell differentiation and ubiquitin-mediated proteolysis (FIG. 5C). While the RNA and protein expression of several key canonical markers was concordantly increased in the Foxp3YFPCre lung Treg, exceptions included the transcription factor Helios, encoded by Ikzf2, whose expression was decreased despite increased transcript levels (FIG. 5D). Further analysis localized the loss of Helios expression to the Notch4+ fraction of the Foxp3YFPCre lung Treg, suggesting either an expansion of Helioslow Notch4+ induced Treg cells, similar to our observation in the asthma model (Harb et al., 2020), or alternatively the loss of Helios expression leading to an increased potential for destabilization (Thornton et al., 2019) (FIG. 5E). The latter results suggest that Notch4 initiated a program of post-translational regulation by pathways such as ubiquitination and proteolysis, both of which were increased in the Treg cells of poly I:C-treated Foxp3YFPCre mice.

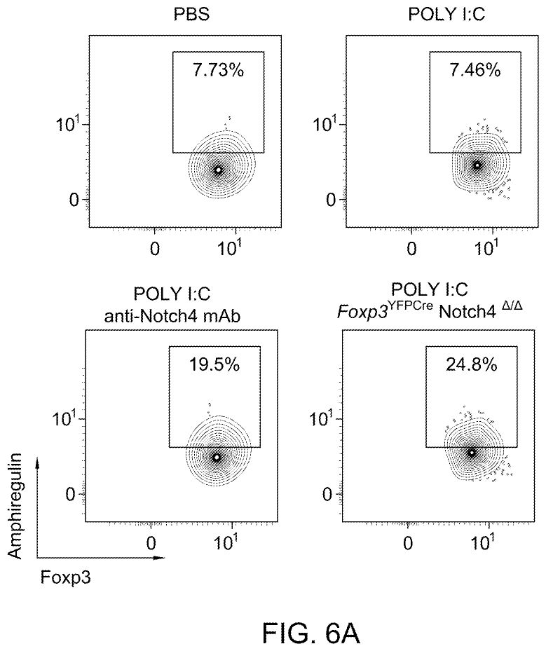

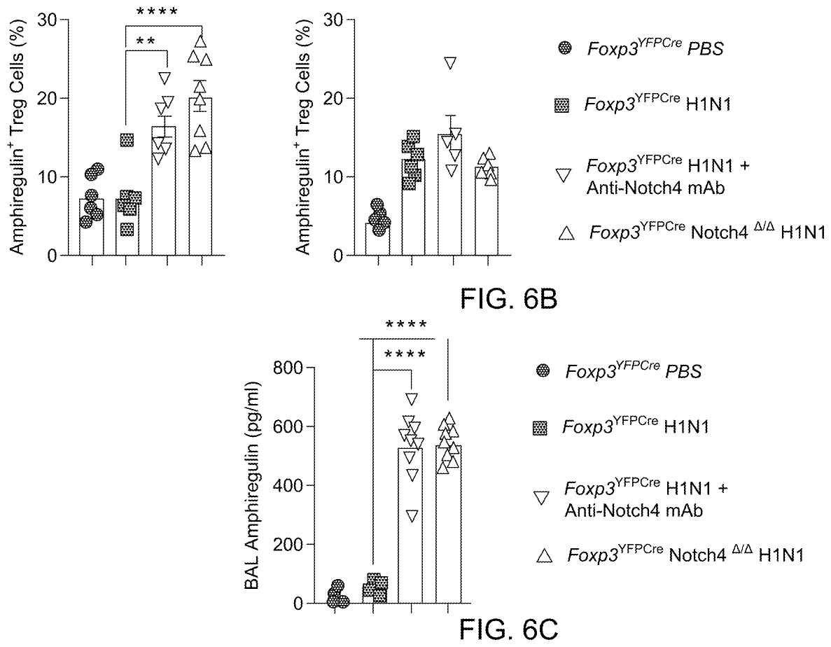

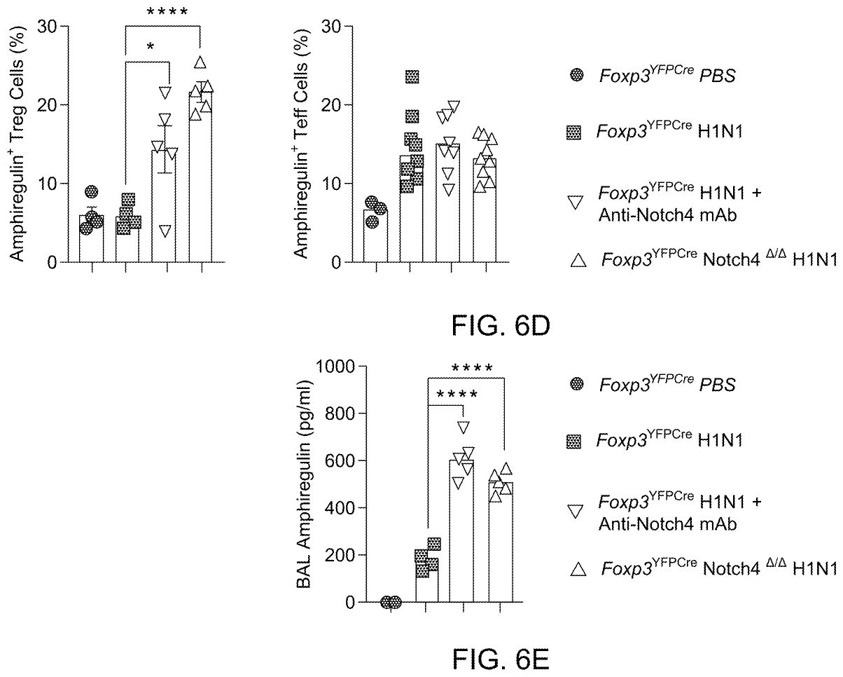

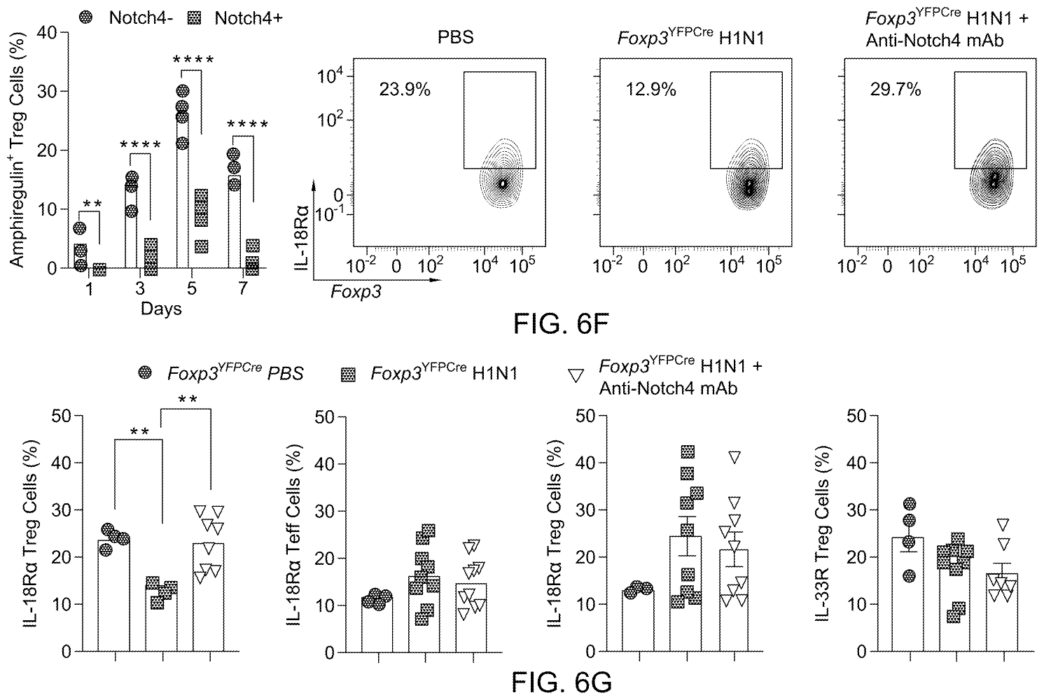

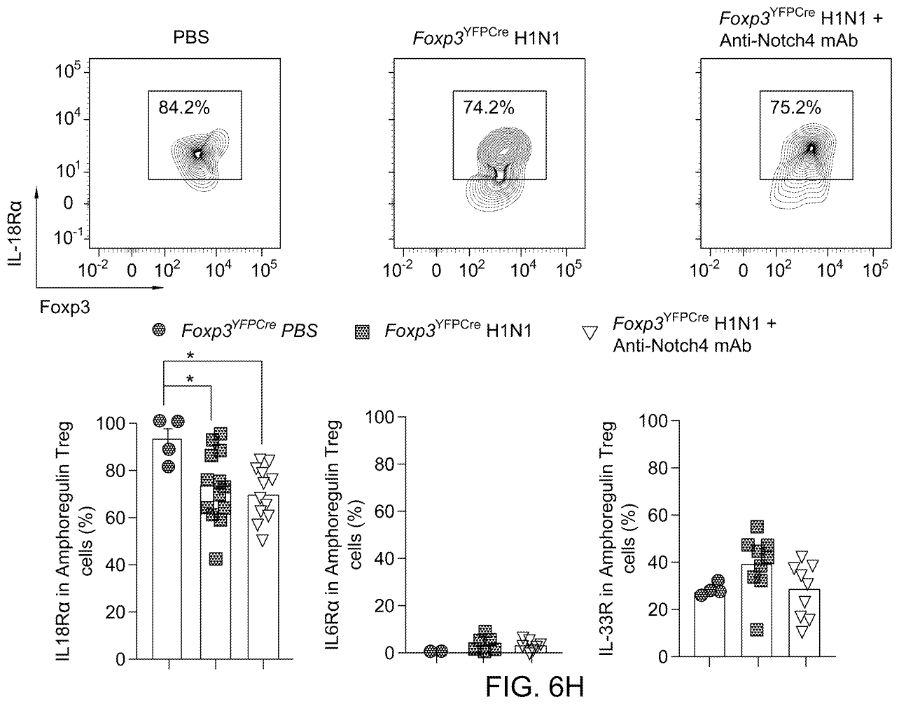

The function of Treg cells in lung tissue repair in H1N1 virus infection involves the production of epidermal growth factor-like cytokine amphiregulin (Arpaia et al., 2015). Treg cells are the main source of amphiregulin in the lung, while Treg cell-specific deletion of Areg, the gene encoding amphiregulin, worsens disease outcome (Arpaia et al., 2015). Analysis of lung Treg cells revealed that despite markedly increased Areg transcripts in Foxp3YFPCre as compared to Foxp3YFPCreNotch4Δ/Δ Treg cells following poly I:C treatment (FIG. 6A), amphiregulin protein expression failed to increase in the Treg cells and BAL fluid of Foxp3YFPCre mice. However, it was markedly increased in lung Treg cells and the BAL fluid of Foxp3YFPCreNotch4Δ/Δ mice and in Foxp3YFPCre mice treated with an anti-Notch4 mAb (FIG. 6A-6C). In contrast, amphiregulin expression in lung tissue Teff cells was unaffected by Notch4 antagonism (FIG. 6B). Similar results were also found in the H1N1 infection model (FIG. 6D, 6E). Consistent with these results, amphiregulin expression was found differentially enriched in Notch4− versus Notch4+ lung Treg cell in Poly I:C treated mice (FIG. 6F). Similar results were also found in lung Treg cells of influenza H1N1 virus-infected mice (data not shown).

Induction of amphiregulin expression in lung Treg cells is mediated primarily by the action of IL-18 with an ancillary effect by IL-33 (Arpaia et al., 2015). We examined the expression of IL-18 receptor alpha chain (IL-18Rct), IL-6Rct chain and IL-33R on lung Treg cells in sham infected versus H1N1 infected mice, the latter without or with anti-Notch4 mAb therapy. Results showed that expression of IL-18Rct on Treg cells declined upon H1N1 infection but recovered on anti-Notch4 mAb treatment. In contrast, IL-18Rct expression on lung tissue Teff cells was unaffected by the anti-Notch4 mAb treatment (FIG. 6G).

Expression of IL-33R on lung Treg cells was unchanged by anti-Notch4 mAb treatment while that of IL-6Rct was increased by Poly I:C treatment (FIG. 6G). Further analysis revealed that most of the amphiregulin expressing Treg cells strongly expressed IL-18Rct and to a lesser extent IL-33R but virtually no IL-6Rct (FIG. 6H). The role of Notch4 in modulating IL-18 induced amphiregulin was analyzed in in vitro experiments employing lung Treg cells isolated from Poly I:C-treated mice. While amphiregulin expression in these Treg cells was minimally induced by IL-18 treatment, it was increased upon co-treatment with anti-Notch4 mAb (FIG. 6I), These results suggested that Notch4 inhibited amphiregulin expression in Treg cells at least in part by antagonizing its upregulation by IL-18.

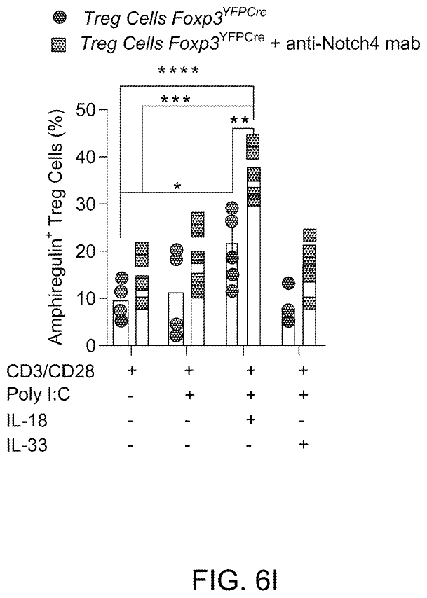

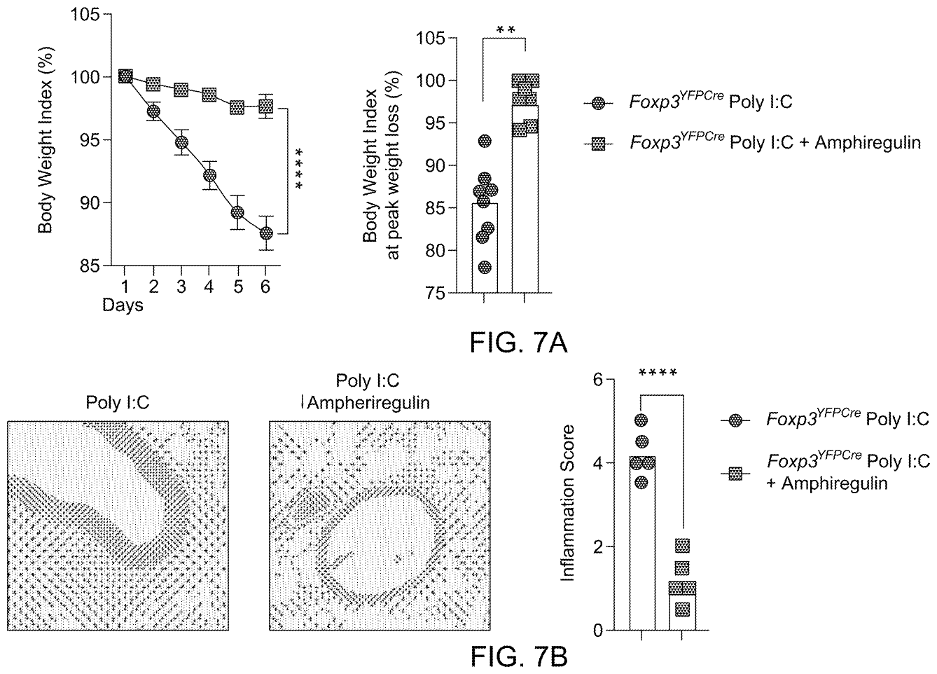

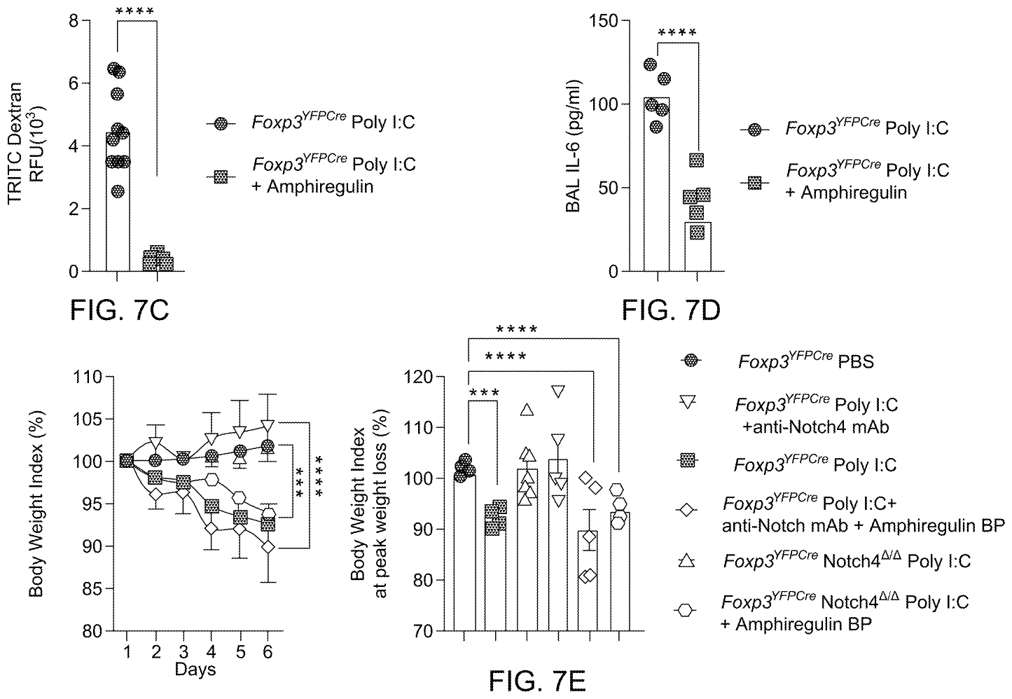

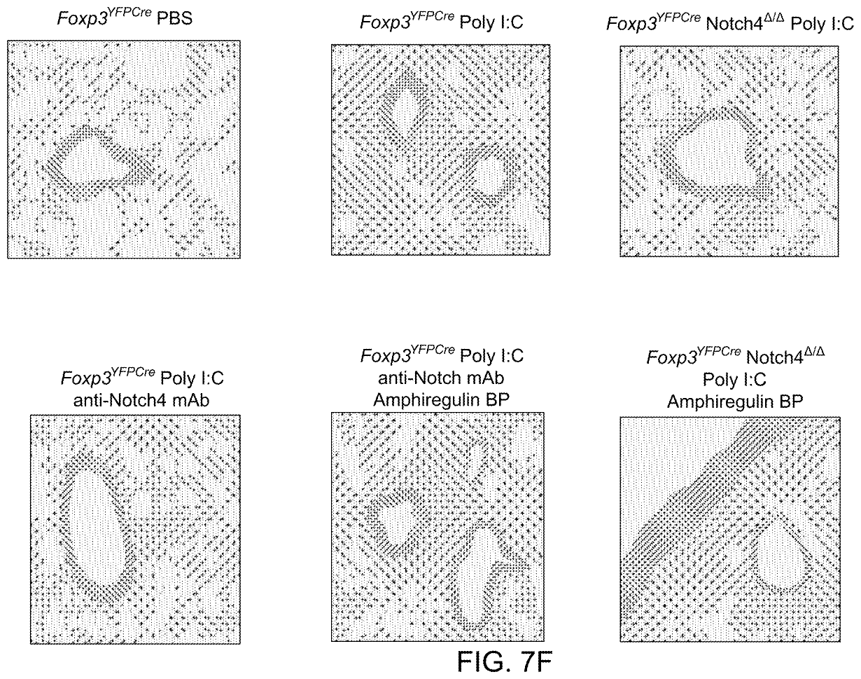

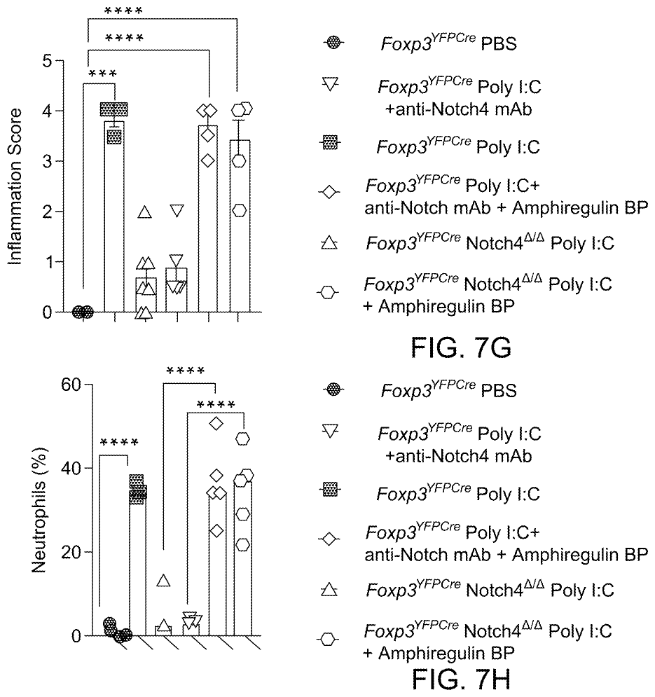

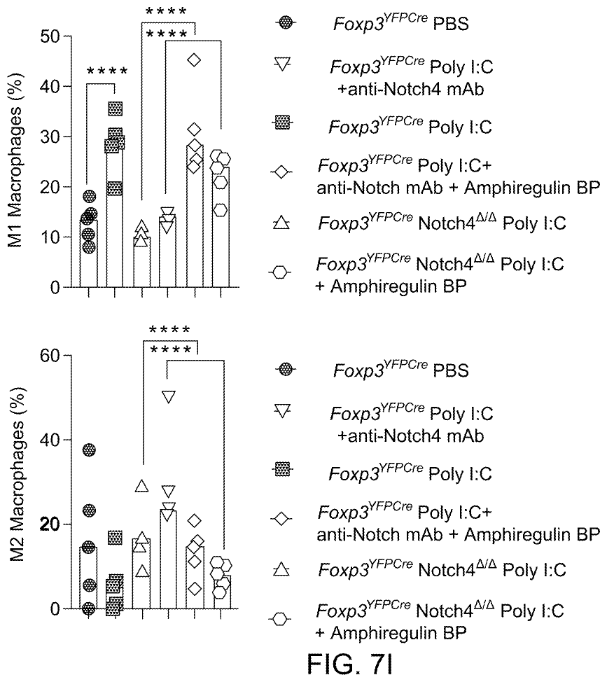

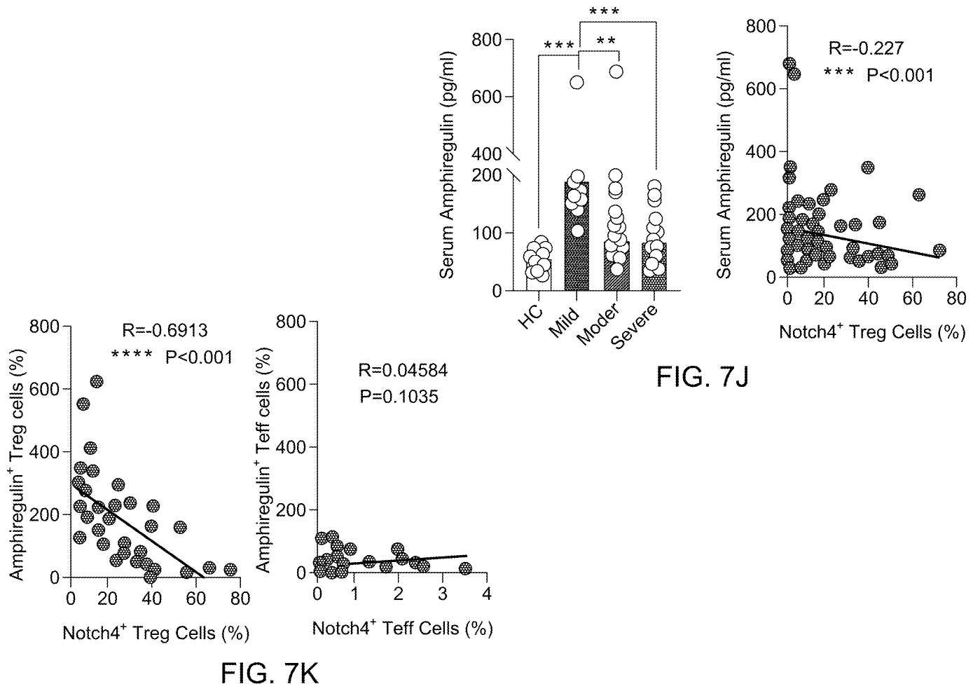

To establish the role of amphiregulin in the protection against lung inflammation induced by Notch4 antagonism and in light of previous studies demonstrating a protective effect of amphiregulin treatment in respiratory infections (Jamieson et al., 2013), we examined the capacity of recombinant amphiregulin therapy to rescue Poly I:C-induced lung inflammation. Treatment of Foxp3YFPCre mice with recombinant amphiregulin largely abrogated the weight loss and the inflammatory responses induced by Poly I:C treatment in association with the restoration of an intact epithelial barrier function (FIG. 7A-7D). Recombinant amphiregulin also reversed the skewing of lung macrophages towards an M1 phenotype upon their in vitro treatment with Poly I:C (FIG. 13H), mirroring the same effect of Foxp3YFPCreNotch4Δ/Δ Treg cells (FIG. 2J).

Reciprocally, we analyzed the impact of co-treatment with an amphiregulin peptide spanning the amino-terminal heparin binding domain, which is necessary for amphiregulin activation of the epidermal growth factor receptor (EGFR) (Johnson and Wong, 1994), on the protective effects of Notch4 antagonism. The peptide was validated to competitively inhibit amphiregulin activation of EGFR as measured by the latter's tyrosine autophosphorylation (FIG. 13C). Results showed that the amphiregulin blocking peptide abrogated the protective effects of Notch4 mAb treatment or Treg cell-specific Notch4 deletion in Poly I:C-induced lung injury, resulting in exacerbated weight loss, tissue inflammation, neutrophilic infiltration and M1 macrophage skewing (FIG. 7E-71). These results were reproduced in experiments utilizing a similarly validated neutralizing anti-amphiregulin mAb (FIG. 13D), which also abrogated the protection imparted by Treg cell-specific Notch4 deficiency against Poly I:C-induced weight loss, lung tissue neutrophil infiltration and increased M1 versus M2 macrophage skewing (Fig. S6E). The anti-amphiregulin mAb co-treatment also reversed the restoration by Notch4 deficiency of airway epithelial barrier integrity, as measured by the increased leakage of high molecular weight dextran instilled in the airways into the blood stream (FIG. 13F).

Consistent with the above results, analysis of the sera of COVID-19 subjects revealed increased amphiregulin concentrations in subjects with mild disease as compared to those of healthy controls, which declined in the sera of moderate and severe patients in a manner inversely proportional to Notch4 expression (FIG. 7J). This inverse correlation between amphiregulin and Notch4 was particularly pronounced when comparing Notch4 and amphiregulin expression specifically in circulating Treg cells (FIG. 7K).

In contrast, there was no correlation between Notch4 and amphiregulin expression on circulating Teff cells (FIG. 7K). Overall, these results are consistent with the suppression of amphiregulin production by Treg cells as a key mechanism of Notch4 inhibitory function in viral infections.

### Discussion

In this study, we have addressed the role of a tissue-specific immunoregulatory mechanism involving inducible Notch4 expression on Treg cells in mediating severe lung tissue inflammation in COVID-19 and related viral infections. We found that Notch4 was increased on circulating Treg cells of COVID-19 patients as a function of disease severity and was associated with heightened mortality, indicating that this mechanism is operative in SARS-CoV-2 infection and may be critically involved in the pathogenesis of acute respiratory failure in this disorder. Furthermore, by employing conventional and humanized immune system mouse models of respiratory viral infections, we found that the evolution of severe viral lung inflammation was contingent on the induction of Notch4 on lung Treg cells, which acted as a molecular switch to alter their regulatory and tissue repair activities. Thus, Notch4 emerges as a critical regulator of disease severity in different respiratory viral infections.

Notch4 was increased on tissue Treg cells very early in lung inflammation and acted to promote the innate immune response at a time point at which adaptive immunity has not yet been effectively mobilized. In particular, the downstream mechanisms by which Notch4 promoted innate immune cell activation in viral infection were different from those involved in promoting the TH2 and TH17 cell adaptive immune responses in asthma, namely the Wnt and Hippo pathways respectively (Geng et al., 2017; Harb et al., 2020). Thus, Treg cell-specific deletion of Yap1 and Wwtr1, encoding the Hippo pathway mediators Yap and Taz, or Ctnnb1, encoding the Wnt pathway effector protein □-Catenin, showed little impact on disease induced by Poly I:C treatment. This bifurcation in the mechanisms by which Notch4 promotes innate versus adaptive immunity points to distinct functions served by this pathway in the respective immune responses. In the context of an acute virus-mediated injury, Notch4 allows for the evolution of an innate immune response instigated by lung epithelium-derived danger signals that is unencumbered by tonic Treg cell suppression (Whitsett and Alenghat, 2015), consistent with the previously noted role for Treg cells in coordinating early protective immunity to viral infections (Lund et al., 2008). This physiological function of Notch4 becomes detrimental in the context of severe respiratory viral infections, resulting in uncontrolled pathological activation of innate immunity, leading to damaging lung tissue injury.

Analysis of Notch4 expression on Treg cells in the context of respiratory viral infection revealed its restriction to lung tissue Treg cells, suggesting a lung tissue origin of circulating Notch4+ Treg cells in human COVID-19 subjects. Induction of Notch4 on lung Treg cells was in part IL-6 dependent, as evidenced by in vitro studies which showed that co-treatment of lung but not splenic Treg cells with IL-6 and poly I:C increased Notch4 expression. These results are consistent with the partial suppression of Poly I:C-induced Notch4 expression and tissue inflammation upon Treg cell-specific deletion of Il6ra. They are also consistent with the positive correlation of increased Notch4 expression in COVID-19 subjects with serum IL-6 concentrations. Notwithstanding these findings, Notch4 but not IL-6 predicted mortality in the patient population. Direct comparison of both genetic and functional interventions that target Notch4 versus IL-6R pathways revealed the superiority of the former approach in mediating protection against Poly I:C and influenza H1N1 virus-induced lung injury. This difference may reflect previously noted additional positive functions of IL-6 in protecting against severe respiratory viral infections separate from its role in Notch4 induction (Dienz et al., 2012; Pyle et al., 2017; Yang et al., 2017).

A key mechanism by which Notch4 impairs lung tissue Treg cell function involved amphiregulin, whose expression is enriched in tissue resident Treg cells especially under conditions of active tissue repair (Arpaia et al., 2015; Burzyn et al., 2013; Schiering et al., 2014; Zaiss et al., 2013; Zaiss et al., 2015). Amphiregulin protein expression was markedly increased in Poly I:C-treated and H1N1 influenza virus-infected mice upon Treg cell-specific Notch4 deletion or treatment with an anti-Notch4 mAb. The protective function of Notch4 antagonism in viral lung injury was reproduced by treatment with recombinant amphiregulin and was abrogated by co-treatment with an amphiregulin blocking peptide or neutralizing antibody. Amphiregulin may thus serve as a key effector mechanism by which lung tissue Treg cells suppress danger signals emanating from the epithelium and other innate immune cells including macrophages. Previous studies by Arpaia et al have shown that Treg cells are the predominant source of amphiregulin in the course of H1N1 infection in the lung. Furthermore, amphiregulin expression specifically in Treg cell is critical for lung tissue repair in the context of H1N1 infection, highlighting the privileged role Treg cell-derived amphiregulin plays in this process (Arpaia et al., 2015). Amphiregulin expression in Treg cells is induced primarily by IL-18 and secondarily by IL-33 (Arpaia et al., 2015). Our studies demonstrated that Notch4 inhibits the capacity of IL-18 to upregulate amphiregulin expression in lung Treg cells, thus providing a mechanism for the suppression of amphiregulin protein expression by Notch4. Amphiregulin mobilization upon Notch4 antagonism would then allow recovery from virus-induced lung injury.

Our results identify the Notch4-amphiregulin nexus as a putative target of therapy in viral respiratory infections including SAR-CoV-2 and Influenza. The remarkable capacity of anti-Notch4 mAb therapy to abrogate Poly I:C and H1N1 influenza virus-induced disease morbidity and mortality in conventional and humanized mice marks Notch4 as an attractive candidate for intervention in severe respiratory viral infections. That Notch4 also plays an essential role in promoting tissue inflammation in asthma further denotes it as a hub common to different inflammatory responses in the lung and which may serve as a target of therapy. Furthermore, the essential role of amphiregulin in mediating the therapeutic outcome of Notch4 inhibition in viral lung injury points to the amphiregulin-epidermal growth factor receptor pathway as an additional locus of intervention. The full spectrum of Notch4-licensed diseases and the role of different downstream pathways in such disorders will require further intensive investigation.

TABLE S1
Demographics of COVID-19 patients stratified by disease severity and predictors of mortality.

| | Overall | | Mild | | Moderate | | Severe | | p-value |
|---|---|---|---|---|---|---|---|---|---|
| n | 118 | | 20 | | 57 | | 41 | | |
| Site, n (%) | | | | | | | | | <0.001 |
| Boston | 38 | (32.2) | 2 | (10.0) | 17 | (29.8) | 19 | (46.3) | |
| Genoa | 44 | (37.3) | 0 | (0.0) | 28 | (49.1) | 16 | (39.0) | |
| Istanbul | 36 | (30.5) | 18 | (90.0) | 12 | (21.1) | 6 | (14.6) | |
| Age, mean (SD) | 63.12 | (18.97) | 42.60 | (15.22) | 65.21 | (17.39) | 70.22 | (15.83) | <0.001 |
| Male, n (%) | 63 | (53.4) | 9 | (45.0) | 25 | (43.9) | 29 | (70.7) | 0.022 |
| White, n (%) | 108 | (91.5) | 18 | (90.0) | 53 | (93.0) | 37 | (90.2) | 0.859 |
| BMI, mean (SD) | 28.57 | (6.88) | 26.94 | (3.94) | 27.98 | (6.05) | 30.18 | (8.69) | 0.151 |
| Any medical problems, n (%) | 95 | (81.2) | 8 | (40.0) | 49 | (86.0) | 38 | (95.0) | <0.001 |
| Immunosuppression | 16 | (13.6) | 1 | (5.0) | 6 | (10.5) | 9 | (22.0) | 0.125 |
| Malignancy | 22 | (18.6) | 1 | (5.0) | 10 | (17.5) | 11 | (26.8) | 0.116 |
| Pulmonary disease | 33 | (28.0) | 2 | (10.0) | 17 | (29.8) | 14 | (34.1) | 0.13 |
| Cardiac disease | 70 | (59.3) | 4 | (20.0) | 32 | (56.1) | 34 | (82.9) | <0.001 |
| Hypertension | 55 | (46.6) | 4 | (20.0) | 24 | (42.1) | 27 | (65.9) | 0.002 |
| Hyperlipidemia | 12 | (10.2) | 0 | (0.0) | 6 | (10.5) | 6 | (14.6) | 0.205 |
| Endocrine disease | 38 | (32.5) | 2 | (10.0) | 19 | (33.3) | 17 | (42.5) | 0.04 |
| Diabetes | 26 | (22.0) | 1 | (5.0) | 14 | (24.6) | 11 | (26.8) | 0.126 |
| COVID-19 treatments | | | | | | | | | |
| Remdesivir | 26 | (22.0) | 0 | (0.0) | 8 | (14.0) | 18 | (43.9) | <0.001 |
| Hydroxychloroquine | 54 | (45.8) | 15 | (75.0) | 24 | (42.1) | 15 | (36.6) | 0.014 |
| Glucocorticoids | 67 | (56.8) | 0 | (0.0) | 35 | (61.4) | 32 | (78.0) | <0.001 |
| Anti-IL-6 | 9 | (7.6) | 0 | (0.0) | 2 | (3.5) | 7 | (17.1) | 0.016 |
| Supportive care | | | | | | | | | |
| Supplemental oxygen | 89 | (75.4) | 9 | (45.0) | 50 | (87.7) | 30 | (73.2) | 0.001 |
| High flow oxygen | 23 | (19.5) | 0 | (0.0) | 0 | (0.0) | 23 | (56.1) | <0.001 |
| Noninvasive ventilation | 8 | (6.8) | 0 | (0.0) | 0 | (0.0) | 8 | (19.5) | <0.001 |
| Mechanical ventilation | 20 | (16.9) | 0 | (0.0) | 0 | (0.0) | 20 | (48.8) | <0.001 |
| ECMO | 2 | (1.7) | 0 | (0.0) | 0 | (0.0) | 2 | (4.9) | 0.148 |
| Dead | 19 | (16.1) | 0 | (0.0) | 5 | (8.8) | 14 | (34.1) | <0.001 |
| 30-day, n (%) | 8 | (6.8) | 0 | (0.0) | 0 | (0.0) | 8 | (19.5) | <0.001 |
| 90-day, n (%) | 18 | (15.3) | 0 | (0.0) | 4 | (7.0) | 14 | (34.1) | <0.001 |
| Biomarkers, mean (SD) | | | | | | | | | |
| Notch4, % | 15.00 | (14.48) | 4.67 | (5.57) | 10.82 | (10.93) | 25.84 | (15.24) | <0.001 |
| IL-6, pg/mL | 89.10 | (209.25) | 124.22 | (172.49) | 43.15 | (38.47) | 137.70 | (326.22) | 0.071 |

| | Crude | | | Adjusted | | P = |
|---|---|---|---|---|---|---|
| | OR | 95% CI | p-value | OR | 95% CI | value |
| Age, years | 1.086 | (1.044, 1.140) | <0.001 | 1.094 | (1.036, 1.177) | 0.005 |
| Male gender | 1.613 | (0.598, 4.654) | 0.354 | 3.713 | (0.942, 17.922) | 0.076 |
| History malignancy | 5.954 | (2.028, 17.718) | 0.001 | 3.717 | (0.950, 15.565) | 0.062 |
| Steroid treatment | 5.020 | (1.552, 22.55) | 0.015 | 5.730 | (1.177, 45.520) | 0.053 |
| Notch4, % | 1.052 | (1.020, 1.088) | 0.002 | 1.046 | (1.008, 1.090) | 0.023 |
| Interleukin-6, pg/mL | 0.999 | (0.992, 1.001) | 0.553 | 1.000 | (0.993, 1.004) | 0.883 |

Disease severity classification. Mild = did not require hospitalization. Moderate = hospitalized but required only supplemental oxygen. Severe = hospitalized and required high flow oxygen, non-invasive ventilation, or mechanical ventilation.
Abbreviations: BMI = body mass index; ICU = intensive care unit; Anti-IL-6 = anti-interleukin-6 monoclonal antibody therapy; ECMO = extracorporeal membrane oxygenation; IL-6 = interleukin-6, OR = Odds Ratio. 95% CI = 95% confidence interval.

### REFERENCES

Arpaia, N., Green, J. A., Moltedo, B., Arvey, A., Hemmers, S., Yuan, S., Treuting, P. M., and Rudensky, A. Y. (2015). A Distinct Function of Regulatory T Cells in Tissue Protection. Cell 162, 1078-1089. Berlin, D. A., Gulick, R. M., and Martinez, F. J. (2020). Severe Covid-19. N Engl J Med. Blanco-Melo, D., Nilsson-Payant, B. E., Liu, W. C., Uhl, S., Hoagland, D., Moller, R., Jordan, T. X., Oishi, K., Panis, M., Sachs, D., et al. (2020). Imbalanced Host Response to SARS-CoV-2 Drives Development of COVID-19. Cell 181, 1036-1045 e1039.
Broggi, A., Ghosh, S., Sposito, B., Spreafico, R., Balzarini, F., Lo Cascio, A., Clementi, N., De Santis, M., Mancini, N., Granucci, F., and Zanoni, I. (2020). Type III interferons disrupt the lung epithelial barrier upon viral recognition. Science 369, 706-712.
Burzyn, D., Kuswanto, W., Kolodin, D., Shadrach, J. L., Cerletti, M., Jang, Y., Sefik, E., Tan, T. G., Wagers, A. J., Benoist, C., and Mathis, D. (2013). A special population of regulatory T cells potentiates muscle repair. Cell 155, 1282-1295.
Charbonnier, L. M., Wang, S., Georgiev, P., Sefik, E., and Chatila, T. A. (2015). Control of peripheral tolerance by regulatory T cell-intrinsic Notch signaling. Nat Immunol 16, 1162-1173.
Copaescu, A., Smibert, O., Gibson, A., Phillips, E. J., and Trubiano, J. A. (2020). The role of IL-6 and other mediators in the cytokine storm associated with SARS-CoV-2 infection. J Allergy Clin Immunol 146, 518-534 e511.
Cucinotta, D., and Vanelli, M. (2020). WHO Declares COVID-19 a Pandemic. Acta Biomed 91, 157-160.
Del Valle, D. M., Kim-Schulze, S., Huang, H. H., Beckmann, N. D., Nirenberg, S., Wang, B., Lavin, Y., Swartz, T. H., Madduri, D., Stock, A., et al. (2020). An inflammatory cytokine signature predicts COVID-19 severity and survival. Nat Med.
Dienz, O., Rud, J. G., Eaton, S. M., Lanthier, P. A., Burg, E., Drew, A., Bunn, J., Suratt, B. T., Haynes, L., and Rincon, M. (2012). Essential role of IL-6 in protection against H1N1 influenza virus by promoting neutrophil survival in the lung. Mucosal Immunol 5, 258-266.
Esty, B., Harb, H., Bartnikas, L. M., Charbonnier, L. M., Massoud, A. H., Leon-Astudillo, C., Visner, G., Subramaniam, M., Phipatanakul, W., and Chatila, T. A. (2019). Treatment of severe persistent asthma with IL-6 receptor blockade. J Allergy Clin Immunol Pract 7, 1639-1642 e1634.
Geng, J., Yu, S., Zhao, H., Sun, X., Li, X., Wang, P., Xiong, X., Hong, L., Xie, C., Gao, J., et al. (2017). The transcriptional coactivator TAZ regulates reciprocal differentiation of TH17 cells and Treg cells. Nat Immunol 18, 800-812.
Hadjadj, J., Yatim, N., Barnabei, L., Comeau, A., Boussier, J., Smith, N., Pere, H., Charbit, B., Bondet, V., Chenevier-Gobeaux, C., et al. (2020). Impaired type I interferon activity and inflammatory responses in severe COVID-19 patients. Science 369, 718-724.
Harb, H., Stephen-Victor, E., Crestani, E., Benamar, M., Massoud, A., Cui, Y., Charbonnier, L. M., Arbag, S., Baris, S., Cunnigham, A., et al. (2020). A regulatory T cell Notch4-GDF15 axis licenses tissue inflammation in asthma. Nat Immunol 21, 1359-1370.
Henderson, L. A., Canna, S. W., Schulert, G. S., Volpi, S., Lee, P. Y., Kernan, K. F., Caricchio, R., Mahmud, S., Hazen, M. M., Halyabar, O., et al. (2020). On the Alert for Cytokine Storm: Immunopathology in COVID-19. Arthritis & rheumatology.
Hermine, O., Mariette, X., Tharaux, P. L., Resche-Rigon, M., Porcher, R., Ravaud, P., and Group, C.-C. (2021). Effect of Tocilizumab vs Usual Care in Adults Hospitalized With COVID-19 and Moderate or Severe Pneumonia: A Randomized Clinical Trial. JAMA Intern Med 181, 32-40.
Investigators, R.-C., Gordon, A. C., Mouncey, P. R., Al-Beidh, F., Rowan, K. M., Nichol, A. D., Arabi, Y. M., Annane, D., Beane, A., van Bentum-Puijk, W., et al. (2021). Interleukin-6 Receptor Antagonists in Critically Ill Patients with Covid-19. N Engl J Med.
Iwasaki, A., and Pillai, P. S. (2014). Innate immunity to influenza virus infection. Nat Rev Immunol 14, 315-328.
Jamieson, A. M., Pasman, L., Yu, S., Gamradt, P., Homer, R. J., Decker, T., and Medzhitov, R. (2013). Role of tissue protection in lethal respiratory viral-bacterial coinfection. Science 340, 1230-1234.
Johnson, G. R., and Wong, L. (1994). Heparan sulfate is essential to amphiregulin-induced mitogenic signaling by the epidermal growth factor receptor. J Biol Chem 269, 27149-27154. Kato, H., Takeuchi, O., Sato, S., Yoneyama, M., Yamamoto, M., Matsui, K., Uematsu, S., Jung, A., Kawai, T., Ishii, K. J., et al. (2006). Differential roles of MDAS and RIG-I helicases in the recognition of RNA viruses. Nature 441, 101-105.
Lowther, D. E., Goods, B. A., Lucca, L. E., Lerner, B. A., Raddassi, K., van Dijk, D., Hernandez, A. L., Duan, X., Gunel, M., Coric, V., et al. (2016). PD-1 marks dysfunctional regulatory T cells in malignant gliomas. JCI Insight 1.
Lucas, C., Wong, P., Klein, J., Castro, T. B. R., Silva, J., Sundaram, M., Ellingson, M. K., Mao, T., Oh, J. E., Israelow, B., et al. (2020). Longitudinal analyses reveal immunological misfiring in severe COVID-19. Nature 584, 463-469.
Lund, J. M., Hsing, L., Pham, T. T., and Rudensky, A. Y. (2008). Coordination of early protective immunity to viral infection by regulatory T cells. Science 320, 1220-1224.
Ostroukhova, M., Qi, Z., Oriss, T. B., Dixon-McCarthy, B., Ray, P., and Ray, A. (2006). Treg-mediated immunosuppression involves activation of the Notch-HES1 axis by membrane-bound TGF-beta. J Clin Invest 116, 996-1004.
Pairo-Castineira, E., Clohisey, S., Klaric, L., Bretherick, A. D., Rawlik, K., Pasko, D., Walker, S., Parkinson, N., Fourman, M. H., Russell, C. D., et al. (2021). Genetic mechanisms of critical illness in COVID-19. Nature 591, 92-98.
Perumalsamy, L. R., Marcel, N., Kulkarni, S., Radtke, F., and Sarin, A. (2012). Distinct spatial and molecular features of notch pathway assembly in regulatory T cells. Sci Signal 5, ra53.
Pyle, C. J., Uwadiae, F. I., Swieboda, D. P., and Harker, J. A. (2017). Early IL-6 signalling promotes IL-27 dependent maturation of regulatory T cells in the lungs and resolution of viral immunopathology. PLoS pathogens 13, e1006640.
Richardson, S., Hirsch, J. S., Narasimhan, M., Crawford, J. M., McGinn, T., Davidson, K. W., and the Northwell, C.-R. C., Barnaby, D. P., Becker, L. B., Chelico, J. D., et al. (2020). Presenting Characteristics, Comorbidities, and Outcomes Among 5700 Patients Hospitalized With COVID-19 in the New York City Area. JAMA.
Rosas, I. O., Brau, N., Waters, M., Go, R. C., Hunter, B. D., Bhagani, S., Skiest, D., Aziz, M. S., Cooper, N., Douglas, I. S., et al. (2021). Tocilizumab in Hospitalized Patients with Severe Covid-19 Pneumonia. N Engl J Med.
Salama, C., Han, J., Yau, L., Reiss, W. G., Kramer, B., Neidhart, J. D., Criner, G. J., Kaplan-Lewis, E., Baden, R., Pandit, L., et al. (2021). Tocilizumab in Patients Hospitalized with Covid-19 Pneumonia. N Engl J Med 384, 20-30.
Salvarani, C., Dolci, G., Massari, M., Merlo, D. F., Cavuto, S., Savoldi, L., Bruzzi, P., Boni, F., Braglia, L., Tuna, C., et al. (2021). Effect of Tocilizumab vs Standard Care on Clinical Worsening in Patients Hospitalized With COVID-19 Pneumonia: A Randomized Clinical Trial. JAMA Intern Med 181, 24-31.
Schiering, C., Krausgruber, T., Chomka, A., Frohlich, A., Adelmann, K., Wohlfert, E. A., Pott, J., Griseri, T., Bollrath, J., Hegazy, A. N., et al. (2014). The alarmin IL-33 promotes regulatory T-cell function in the intestine. Nature 513, 564-568.
Thornton, A. M., Lu, J., Korty, P. E., Kim, Y. C., Martens, C., Sun, P. D., and Shevach, E. M. (2019). Helios(+) and Helios(−) Treg subpopulations are phenotypically and functionally distinct and express dissimilar TCR repertoires. Eur J Immunol 49, 398-412.
Toniati, P., Piva, S., Cattalini, M., Garrafa, E., Regola, F., Castelli, F., Franceschini, F., Foca, E., Andreoli, L., Latronico, N., et al. (2020). Tocilizumab for the treatment of severe COVID-19 pneumonia with hyperinflammatory syndrome and acute respiratory failure: A single center study of 100 patients in Brescia, Italy. Autoimmun Rev, 102568.
Vabret, N., Britton, G. J., Gruber, C., Hegde, S., Kim, J., Kuksin, M., Levantovsky, R., Malle, L., Moreira, A., Park, M. D., et al. (2020). Immunology of COVID-19: current state of the science. Immunity.
Veiga, V. C., Prats, J., Farias, D. L. C., Rosa, R. G., Dourado, L. K., Zampieri, F. G., Machado, F. R., Lopes, R. D., Berwanger, O., Azevedo, L. C. P., et al. (2021). Effect of tocilizumab on clinical outcomes at 15 days in patients with severe or critical coronavirus disease 2019: randomised controlled trial. BMJ 372, n84.
Verma, M. K., Clemens, J., Burzenski, L., Sampson, S. B., Brehm, M. A., Greiner, D. L., and Shultz, L. D. (2017). A novel hemolytic complement-sufficient NSG mouse model supports studies of complement-mediated antitumor activity in vivo. J Immunol Methods 446, 47-53.
Whitsett, J. A., and Alenghat, T. (2015). Respiratory epithelial cells orchestrate pulmonary innate immunity. Nat Immunol 16, 27-35.
Winkler, E. S., Bailey, A. L., Kafai, N. M., Nair, S., McCune, B. T., Yu, J., Fox, J. M., Chen, R. E., Earnest, J. T., Keeler, S. P., et al. (2020). SARS-CoV-2 infection of human ACE2-transgenic mice causes severe lung inflammation and impaired function. Nat Immunol.
Woodham, A. W., Zeigler, S. H., Zeyang, E. L., Kolifrath, S. C., Cheloha, R. W., Rashidian, M., Chaparro, R. J., Seidel, R. D., Garforth, S. J., Dearling, J. L., et al. (2020). In vivo detection of antigen-specific CD8(+) T cells by immuno-positron emission tomography. Nature methods.
Xia, M., Harb, H., Saffari, A., Sioutas, C., and Chatila, T. A. (2018). A Jagged 1-Notch 4 molecular switch mediates airway inflammation induced by ultrafine particles. J Allergy Clin Immunol 142, 1243-1256 e1217.
Xia, M., Viera-Hutchins, L., Garcia-Lloret, M., Noval Rivas, M., Wise, P., McGhee, S. A., Chatila, Z. K., Daher, N., Sioutas, C., and Chatila, T. A. (2015). Vehicular exhaust particles promote allergic airway inflammation through an aryl hydrocarbon receptor-notch signaling cascade. J Allergy Clin Immunol 136, 441-453.
Xu, X., Han, M., Li, T., Sun, W., Wang, D., Fu, B., Zhou, Y., Zheng, X., Yang, Y., Li, X., et al. (2020). Effective treatment of severe COVID-19 patients with tocilizumab. Proc Natl Acad Sci USA. Yang, M. L., Wang, C. T., Yang, S. J., Leu, C. H., Chen, S. H., Wu, C. L., and Shiau, A. L. (2017). IL-6 ameliorates acute lung injury in influenza virus infection. Sci Rep 7, 43829.
Zaiss, D. M., van Loosdregt, J., Gorlani, A., Bekker, C. P., Grone, A., Sibilia, M., van Bergen en Henegouwen, P. M., Roovers, R. C., Coffer, P. J., and Sijts, A. J. (2013). Amphiregulin enhances regulatory T cell-suppressive function via the epidermal growth factor receptor. Immunity 38, 275-284.
Zaiss, D. M. W., Gause, W. C., Osborne, L. C., and Artis, D. (2015). Emerging functions of amphiregulin in orchestrating immunity, inflammation, and tissue repair. Immunity 42, 216-226. Zhou, F., Yu, T., Du, R., Fan, G., Liu, Y., Liu, Z., Xiang, J., Wang, Y., Song, B., Gu, X., et al. (2020a). Clinical course and risk factors for mortality of adult inpatients with COVID-19 in Wuhan, China: a retrospective cohort study. Lancet 395, 1054-1062.
Zhou, Z., Ren, L., Zhang, L., Zhong, J., Xiao, Y., Jia, Z., Guo, L., Yang, J., Wang, C., Jiang, S., et al. (2020b). Heightened Innate Immune Responses in the Respiratory Tract of COVID-19 Patients. Cell Host Microbe 27, 883-890 e882.

### Example 2

Experimental Model and Subject Details

Human subjects. We recruited three prospective cohorts of patients with COVID-19 from the following institutions: (1) Massachusetts General Hospital in Boston, Massachusetts, USA; (2) San Martino Hospital in Genoa, Italy; (3) Istanbul Medical Faculty Hospitals in Istanbul, Turkey. Inclusion criterion included a clinical syndrome consistent with COVID-19 disease and a positive SARS-CoV-2 test from a nasal swab based on RT-PCR. Severity of illness was defined as follows: (1) Mild for patients who did not require inpatient hospitalization; (2) Moderate for patients requiring inpatient hospitalization and who did not require therapies for acute respiratory failure such as high flow oxygen (defined as a flow rate of more than 15 liters per minute), non-invasive positive pressure ventilation, mechanical ventilation, and who did not require therapies for other types of organ failure such as renal replacement therapy or shock; (3) Severe for patients with organ failure requiring supportive therapies typically administered in the intensive care unit such as high flow oxygen, non-invasive positive pressure ventilation, mechanical ventilation, vasopressors, renal replacement therapy; (4) Convalescent for patients who have recovered from their acute illness and discharged from the hospital. A cohort of country-matched healthy controls were also recruited (Supplementary Data Table 1).

Clinical data including patient characteristics, therapies received, and clinical outcomes was abstracted from the medical record into a password-protected REDCap (Research Electronic Data Capture) database. A peripheral blood draw was obtained at the time of enrollment and weekly thereafter (if available) for flow cytometry and cytokine analysis.

Study approval. Recruitment of Boston study participants was approved by the Institutional Review Board at Massachusetts General Hospital and Boston Children's Hospital. Recruitment of Genoa study participants was approved by the Institutional Review Board at Genoa University. Recruitment of Istanbul study participants was approved by the Institutional Review Board at Istanbul University. All animal studies were reviewed and approved by the Boston Children's Hospital office of Animal Care Resources and Massachusetts General Hospital office of Animal Care Resources.

Mice. The following mouse strains were obtained from the JAX Laboratories: Foxp3YFPCre (B6.129(Cg)-Foxp3tm4(YFP/icre)Ayr/J)⁴², Il6^{fl/fl} (B6; SJL-R6ratm1.1Drew/J)⁴³, Notch1 fl/fl (B6.129X1-Notch1tm2Rko/Grid/J)⁴⁴, Notch2^{fl/fl} (B6.1295-Notch2tm3Grid/J)⁴⁵, Notch3fl/fl Wwtr1^{fl/fl} (B6.129(Cg)-Wwtr1^{tm1Hmc}/J) and Yap1^{fl/fl} (B6.129P2(Cg)-Yap1tm1.1Dupa/J)^{46,47}, NOD-Prkdc^{scid}Il2rg^{tmiw}ⱼ¹/Sz (NSG)²⁷. and (Ctnnb1^{fl/fl}) (B6(Cg)-Ctnnb1tm1Knw/J)⁴⁸ mice. Notch4^{fl/fl} (Notch4tm1c(NCOM)Mfgc) were obtained from the Canadian mutant mouse repository. Rbpj1^{fl/fl} (B6.129P2-Rbpj^{tm1Hon}/HonRbrc) were kind gifts of Pamela Stanley and Tasuku Honjo, respectively^{49,50} Notch3 floxed allele mice (Notch3^{fl/fl}) were derived by Crispr/Cas 9 targeting with LoxP sequences inserted in introns 4 and 9 to target exons 5-9 by Cre-mediated excision.

Regulatory T-cell co-cultures with macrophages. Macrophages cells were isolated from lungs of Poly I:C-treated mice. Macrophages were isolated based on CD45⁺ F4/80⁺ MHCII⁺. Macrophages were then co-cultured (at 1:1 concentration) with lungs Treg cells from Foxp3^{YFPCre} or Foxp3^{YFPCre}Notch4^{Δ/Δ} mice Poly I:C-treated mice. Cells were treated with Poly I:C at a concentration of 10 ug/ml for three days. After 72 h, M1 (F4/80⁺ MHCII⁺ CD68⁺ CD80⁺CD86⁺) and M2 (F4/80⁺ MHCII⁺ CD163⁺ CD206⁺) polarization was measured by flow cytometric analysis.

In vitro Macrophages—Amphiregulin co-cultures. Macrophages cells were isolated from the lungs of Poly I:C treated Foxp3^{YFPCre} mice. These cells were incubated with recombinant Amphiregulin (R&D) (10 μg/ml). 72 hours later, the expression of different M1 and M2 markers were measured by flow cytometry.

In vitro Notch4 induction: Lung and spleen Treg cells from Poly I:C or PBS-treated Foxp3^{YFPCre} mice were isolated by cell sorting (Sony Sorter, MA900). Treg cells were seeded at 1×10⁴ cells in 96-well plates then stimulated with CD3/CD28 Dynabeads (ThermoFisher) in presence of Poly I:C (10 μg/ml) alone or in combination with recombinant IL-6 and IL-33 (10 μg/ml; Peprotech) for 72 h. Notch4 expression on Foxp3⁺ Treg cells was then assessed by Flow cytometry.

In vitro amphiregulin induction: Lung Treg cells from Poly I:C or PBS-treated Foxp3^{YFPCre} mice were seeded at 1×10⁴ cells then stimulated with CD3/CD28 Dynabeads with Poly I:C (10 μg/ml) either alone or in combination with recombinant IL-18 and IL-33 (R&D systems, Peprotech, respectively, 10 μg/ml) for 72 h. Amphiregulin expression was then assessed by Flow cytometry.

Mouse anti-human Notch4 monoclonal antibody generation. Notch4-deficient mice were immunized with 50 micrograms of recombinant Notch4-His protein (Sino-Biologicals, Wayne, PA) suspended in Dulbecco's phosphate buffered saline (PBS; GIBCO, Grand Island, NY) and emulsified with an equal volume of complete Freund's adjuvant (Sigma Chemical Co., St. Louis, MO). Fourteen days after the initial immunization, the mice were given a booster immunization i.p. with 25 μg of recombinant Notch4-His protein suspended in PBS and emulsified with an equal volume of incomplete Freund's adjuvant. A second booster of 25 μg of recombinant Notch-His protein in PBS was given after another 14 days. The mice were re-boosted 2× with 25 μg of recombinant Notch4-His protein to raise their antibody titers, then the mouse with the best titer was rested for 3 weeks from the last immunization, then boosted by intraperitoneal and subcutaneous injection of 25 ug recombinant Notch4-His protein in PBS. Three days later the mouse was euthanized, and the spleen and lymph nodes were collected and made into a cell suspension, then washed with DMEM. The spleen/lymph node cells were counted and mixed with SP 2/0 myeloma cells (ATCC No. CRL8-006, Rockville, MD) that are incapable of secreting either heavy or light chain immunoglobulin chains 51 using a spleen:myeloma ratio of 2:1. Cells were fused with polyethylene glycol 1450 (ATCC) in eight 96-well tissue culture plates in HAT selection medium according to standard procedures 52. Between 10 and 21 days after the fusions, hybridoma colonies became visible and culture supernatants were harvested then screened by ELISA for Notch4. Potential positive wells were expanded and re-screened against Notch1-His, Notch2-His, Notch3-His/Gst, Notch4-His and an irrelevant protein by ELISA. Hybridomas showing specificity for Notch4 alone were identified and subcloned twice by limiting dilution to obtain stable, clonal hybridomas.

Isolation of Human peripheral blood mononuclear cells (PBMCs). Human PBMCs were isolated from whole blood from either healthy control, mild COVID-19, moderate COVID-19 or severe COVID-19 probands via density gradient using Ficoll (GE Healthcare). PBMCs were then stored frozen in Fetal Calf Serum (FCS) (Sigma Aldrich) and 15% Dimethyl sulfoxide (DMSO) (Sigma Aldrich). The cells were later thawed for analysis of their Notch, Yap1, β-Catenin or different chemokine and cytokine expression by flow cytometry.

IL-6 and Amphiregulin ELISA. Genoa patients IL-6 concentrations were measured using ELISA (R&D, USA) per manufacturer's protocol. As for Amphiregulin, Amphiregulin concentrations for the whole cohort were measured using Amphiregulin ELISA kit (R&D, USA).

Cytokine measurements. IL-113, IL-6 (For Boston and Istanbul cohorts), IL-8, IFNα, IFNβ, IFNγ, IFNλ, CXCL10 and TNF were measured using Legendplex (Biolegend) per manufacturer's protocol. Mouse cytokine were assessed using Legendplex (Biolegend) per manufacturer's protocol on BAL samples from Poly I:C treated mice (Day 7) or on BAL samples from H1N1-infected mice (Day 12). OSM, LIF and CXCL5 were also measured on the same samples using Quantikine Elisa kits (R&D systems).

Polyinosinic-polycytidylic acid (Poly I:C) mouse model. Mice were treated intratracheally with 2.5 mg/kg of Poly I:C HMW (InvivoGen) daily for six consecutive days. The weight of the mice was recorded daily upon application of the Poly I:C. The mice were subjected to airway hyperresponsiveness at day 7, then euthanized and analyzed. For blockading amphiregulin, mice were treated with a peptide spanning amino acids 91140 of the middle region of the human amphiregulin preproprotein (amphiregulin91-140 peptide; Mybiosource). The peptide was given intratracheally at 10 μg/ml in PBS in a final volume of 100 μl. For the amphiregulin neutralizing antibody tests, the mice were given intraperitoneally 20 μg of goat anti-mouse amphiregulin mAb (clone AF989; R&D systems) or isotype control mAb (clone AB-108-C; R&D systems) daily for the duration of the experiment.

H1N1 influenza A virus preparation. Mouse-adapted H1N1 Influenza A virus (PR/8/34) was obtained from Charles River (Catalogue no. 10100374). Viral stocks were calculated to contain 40,000 infectious units (IU) per mouse and were diluted to a volume of 20 μl/mouse in PBS. For lethal dose (LD75), the viral stocks were calculated to contain 60,000 IU per mouse and diluted to a volume of 20 μl/mouse in PBS as well.

H1N1 Influenza A viral infection model. Mice were treated intranasally on day 0 of the experiments with either a 40,000 pfu dose of the H1N1 virus or 70,000 pfu dose, equivalent to a lethal dose 75 (LD75), as indicated. The mice were monitored on a daily basis to see signs of infection. The weights of the mice were recorded and once a mouse weight loss exceeded 20-25%, the mouse was euthanized. The endpoint of the experiments was set at day 12, to capture the peak of inflammation. For antibody treatment experiments, mice received anti-Notch4 mAb (10 μg/ml) (clone HMN-4-14; Bio-X-Cell), anti-IL-6Rα mAb (10 μg/ml) (clone 15A7, Bio-X-Cell) or their respective isotype control antibodies, as indicated.

Humanized mice H1N1 viral infection. NOD-Prkdc^{scid}Il2rg^{tmiw}ⱼ¹/Sz (NSG) humanized mice were reconstituted with PBMCs from healthy control. Then, mice were treated with a sublethal dose of the virus as indicated intranasally on day 0. The mice were monitored on a daily basis to see signs of infection. The weights of the mice were recorded and once a mouse weight loss exceeded 20-25%, the mouse was euthanized. For sublethal experiments, the endpoint of the experiment was set at day 12, to capture the peak of inflammation.

Viral Load. Viral titers were determined by quantification of viral transcripts using reverse transcription (RT)-quantitative (q) PCR as previously described 53-55 The collected lung tissue was weighed, and total RNA was isolated using RNAeasy mini kit (Qiagen). Purified RNA was converted into cDNA using the SuperScript™ III First-Strand Synthesis System (Invitrogen) following the manufacturer's recommended procedures. Quantification of viral load was performed in 96-well plates and run on a QuantStudio 3 Real-Time PCR instrument (Applied Biosystems).

The amplification was carried out using H1N1 nucleoprotein region specific primers:
forwardprimer, 5′-GGGGGAAGCTCTCCACTAGA-3′ (SEQ ID NO: 54) and reverse primer, 5′-GTTGTGTGCTGATTTGGCCC-3′ (SEQ ID NO: 55) in the presence of the Power SYBR Green Master Mix (Applied Biosystems). The PCR amplification cycle was set as 95° C. for 5 min for initial denaturation, followed by 50 cycles of 95° C. for 15 sec, 60° C. for 1 min. The standard curve was generated using 10-fold serial dilutions of constructed plasmid pcDNA3-NP (Addgene) by plotting each cycle threshold (CT) value against the log quantity of each standard plasmid copy number. Viral RNA copies were extrapolated from the standard curve using the sample CT value and represented as viral RNA copies per gram of tissue. For determination of the viral load in the humanized mouse model, viral transcripts were quantitated using reverse transcription (RT)-quantitative (q) PCR as previously described 53-55. Briefly, RNA was isolated from right upper lung lobe homogenates prepared in TRIzol (Sigma-Aldrich) and 1 μg RNA as determined by a Nanodrop 2000 spectrophotometer (ThermoFisher) was converted to cDNA by reverse transcription using the protocol provided by the manufacturer (TaqMan Reverse Transcription Reagents, ThermoFisher). qPCR reactions in the presence of SYBR Green (FastStart Essential DNA Green Master Mix, Roche) were performed on a Light-Cycler 96 Instrument (Roche). Primers: Polymerase gene (PA), 5′-CGGTCCAAATTCCTGCTGA-3′ (SEQ ID NO: 56) (forward) and 5′-CATTGGGTTCCTTCCATCCA-3′ (SEQ ID NO: 57) (reverse).

Measurement of airway functional responses. Poly I:C-induced airway hyperreactivity (AHR) was measured, as previously described 16. Anesthetized mice were exposed to doubling concentrations of aerosolized acetyl-β-methacholine (Sigma-Aldrich) by using a Buxco small-animal ventilator (Data Sciences International). The relative peak airway resistance for each methacholine dose, normalized to the saline baseline, was calculated.

Lung histopathology staining. Paraffin-embedded lung sections were stained with hematoxylin and eosin (H&E) or Paraffin-acid-Schiff staining (PAS). The lung pathology was scored by blinded operators. Inflammation was scored separately for cellular infiltration around blood vessels and airways: 0, no infiltrates; 1, few inflammatory cells; 2, a ring of inflammatory cells 1 cell layer deep; 3, a ring of inflammatory cells 2-4 cells deep; 4, a ring of inflammatory cells of >4 cells deep⁵⁶ A composite score was determined by the adding the inflammatory scores for both vessels and airways.

Dextran Leakage Assay: 25 μg of TRITC-Dextran (MW 40,000; Sigma) in 80 μl saline was instilled intratracheally in each mouse. After one hour of dextran instillation, blood from the mice were collected retro-orbitally in sodium citrate solution, and the plasma was separated by centrifugation (2000 rpm, 15 minutes at 4 C). Leakage of dextran in the bloodstream is calculated as TRITC fluorescence in the plasma compared to plasma from mock-treated mice.

Flow cytometric analysis of mouse and human cells. Antibodies against the following murine antigens were used for flow cytometric analyses: IL-4 (clone 11B11, catalogue no: 504104 1:300 dilution, Biolegend), Foxp3 (FJK-165, catalogue no: 48-5773-82 1:300, Thermofisher), IFN□ (XMG1.2, catalogue no: 505825 1:300, Biolegend), IL-13 (eBio13a, catalogue no.: 47-7133-82 1:300, Thermofisher), TNF (MP6-XT22, Catalogue no: 554419, 1:200, BD Biosciences), Helios (22F6, catalogue no: 47-9883-42 1:200, Thermofisher, CD11c (N418, catalogue no: 117318 1:500, Biolegend), CD11b (M1/70, catalogue no: 101228 1:500, Biolegend), CD4 (GK1.5, catalogue no: 100451, 1:500, Biolegend), CD206 (C068C2, Catalogue no: 141719, 1:300, Biolegend), CD163 (S150491, Catalogue no: 155305 1:200, Biolegend), CD68 (FA-11, Catalogue no: 137023 1:200, Biolegend), CD80 (16-10A1, Catalogue no: 104711 1:200, Biolegend), F4/80 (BM8, Catalogue no: 48-4801-82 1:300, Thermofisher), MHCII (39-10-8, Catalogue no: 115006, 1:300, Biolegend), IL-33Ralpha (DIH9, Catalogue no: 145312 1:300, Biolegend), Lineage (anti-CD3/GR-1/CD11B/CD45-B220/TER 119) (Biolegend, 1:300 catalogue no: 78022), CD3 (17A2, catalogue no: 100203, 1:500, Biolegend), IL-17 (TC11-18H10.1, catalogue no: 506922, 1:200, Biolegend), GR-1 (RB6-8C5, catalogue no: 108406, 1:500, Biolegend), CD45 (30-F11, catalogue no: 103140, 1:300, Biolegend), Notch4 (HMN4-14, catalogue no: 128407 1:400, Biolegend), CD134 (DX-86, catalogue no:17-1341-82, 1:400 Thermofisher), CTLA-4 (CD152) (UC10-4B9, catalogue no: 106310, 1:400 Biolegend), CD223 (Ebio-C9B7W, catalogue no:12-2231-83, 1:400 Thermofisher), CD279 (J43, catalogue no:12-9985-82, 1:400 Thermofisher), IL-6Rα (15A7, catalogue no: BE0047, 1:200 Bio-X-Cell), Jagged1 (CD339) (HMJ1-29, catalogue no:130908, 1:400 Biolegend), Amphiregulin polyclonal antibody (orb7378, 1:200, Biorbyt), IL-6Rα (D7715A7, catalogue 115813, 1:400 Biolegend), IL-18Ra (BG/IL18RA, catalogue 132903, 1:400 Biolegend), P-EGFR(D7A5, catalogue no:D7A5, 1:200 Cell Signaling Technology), Anti-CD16/CD32 (93, catalogue no: 101319, 1:1000 Biolegend). Antibodies against the following human antigens were used: CD3 (HIT3a, catalogue no: 300318, 1:200, Biolegend), CD4 (RPA-T4, catalogue no: 300530, 1:200, Biolegend), Foxp3 (PCH-101, catalogue no: 48-4776-42,56-4716-41, 1:100 Thermofisher), Helios (22F6, catalogue no: 47-9883-42 1:100, Thermofisher), Notch1 (HMN1-519, catalogue no: 566023, 1:300, BD Pharmingen), Notch2 (HMN2-25, catalogue no: 742291, 1:300, BD Pharmingen), Notch3 (HMN3-21, catalogue no: 744828, 1:300, BD Pharmingen), Notch4 (HMN4-2, Catalogue no: 563269, 1:300, BD Pharmingen), Yap1 (D8H1X, Catalogue no: 14729S, 1:100, Cell Signaling Technology), beta-Catenin (196624, Catalogue no: IC13292V, 1:200, R&D system), CD25 (BC96, Catalogue no: 12-0259-42 1:300, Thermofisher), IL-4 (MP4-25D2, Catalogue no: 500828 1:200, Biolegend), IL-13 (JES10-5A2, Catalogue no: 501912, 1:200, Biolegend), CCR4 (L291H4, Catalogue no: 359417 1:300, Biolegend), CXCR3 (G025H7, Catalogue no: 353708, 1:300 Biolegend), CRTH2 (BM16, Catalogue no: 350108 1:300, Biolegend), CD127 (A019D5, Catalogue no: 351320 1:300, Biolegend), IFNγ (4S. B3, Catalogue no: 560741 1:200, BD Biosciences) and Amphiregulin polyclonal antibody (orb7378 1:200, Biorbyt), CD196 (G034E3, Catalogue no: 353423, 1:300 Biolegend). The specificity and optimal dilution of each antibody was validated by testing on appropriate negative and positive controls or otherwise provided on the manufacturer's website. Intracellular cytokine staining was performed as previously described 57. Dead cells were routinely excluded from the analysis based on the staining of eFluor 780 Fixable Viability Dye (1:1000 dilution) (Thermofisher). Stained cells were analyzed on a BD LSR Fortessa cell analyzer (BD Biosciences) and data were processed using Flowjo (Tree Star Inc.).

M1 and M2 gating strategy. M1 was defined as follows: CD45⁺CD4⁻ F4/80⁺ MHCII⁺ CD68⁺ CD80⁺CD86⁺ while M2 macrophages were defined as follows: CD45⁺CD4⁻F4/80⁺ MHCII+CD163+CD206+.

Transcriptome Profiling. Treg cells were isolated from either Foxp3^{YFPCre} or Foxp3YFPCre Notch4^{Δ/Δ} mice that were treated with Poly I:C as shown above. mRNA was isolated using Qiagen RNeasy mini kit (Qiagen). RNA was then converted into double-stranded DNA (dsDNA), using SMART-Seq v4 Ultra Low Input RNA kit (Clontech). dsDNA was then fragmented to 200-300 bp size, using M220 Focused-ultrasonicator (Covaris), and utilized for construction of libraries for Illumina sequencing using KAPA Hyper Prep Kit (Kapa Biosystems). Libraries were then quantified using Qubit dsDNA HS (High Sensitivity) Assay Kit on Agilent High Sensitivity DNA Bioanalyzer.

Gene-level read counts were quantified using feature Counts and the latest Ensambl mouse annotation (GRCm38. R101). Raw data were trimmed using Trimmomatic (version 0.39, default parameters), tool for Illumina NGS data. To identify differentially expressed genes, we used 3 algorithms: DESeq2 (version 1.26.0), edgeR (version 3.28.1) and Lima (3.42.2) Bioconductor packages with default parameters. Count tables were normalized to TPM (Transcripts per Million) for visualizations and QC. Sample clustering, path analyses and integration of the results were performed using a custom-made pipeline available upon request (Variant Explorer RNAseq). Transcripts were called as differentially expressed when the adjusted p values were below 0.05, fold-changes over ±1.5 and false discovery rate (FDR) were below 0.05. For our path analyses, we tested 10,715 biological pathways from KEGG and GO annotations. We filtered the results using an adjusted p value below 0.01.

Statistical analysis. Student's two-tailed t-test, one- and two-way ANOVA and repeat measures two-way ANOVA with post-test analysis and log-rank test of groups were used to compare test groups, as indicated. Linear Regression was used for correlation analysis. For analysis of the human data, summary statistics were calculated using number (percentage) for binary and categorical data and mean (standard deviation) or median (interquartile range) for continuous data depending on the normality of the distribution. Logistic regression was performed to determine the association between Notch4 expression and mortality. Covariates including age, gender, and study site were selected based on a priori knowledge. In the case of highly correlated covariates, we chose the predictor that had the strongest univariate association between the potential predictor and outcome. In the final model, the outcome was death at any time after study enrollment, the predictors of interest were Notch4 expression and serum interleukin-6 levels, and covariates included age, gender, history of malignancy, and corticosteroid treatment. Analyses were performed in R version 3.6.1. Two-sided p-values of <0.05 were considered statistically significant.

What is claimed is:

1. An antibody or antigen binding fragment that binds to Notch4 and comprises:
a) a heavy chain variable region (V_{H}) comprising an amino acid sequence having at least 85% identity to an amino acid sequence selected from the group consisting of SEQ ID NOs 2, 4, or 6; or
b) a heavy chain variable region (V_{H}) comprising an amino acid sequence encoded by a nucleotide sequence having at least 85% identity to a nucleotide sequence selected from the group consisting of SEQ ID NOs 3, 5, or 7; or
c) a light chain variable region (V_{L}) comprising an amino acid sequence having at least 85% sequence identity to an amino acid sequence selected from the group consisting of SEQ ID NOs 8, 10, 12 or 14; or
d) a light chain variable region (V_{L}) comprising an amino acid sequence encoded by a nucleotide sequence having at least 85% identity to a nucleotide sequence selected from the group consisting of SEQ ID NOs 9, 11, 13, or 15.

2. The antibody or antigen binding fragment of claim 1, wherein the antibody or antigen-binding fragment comprises a nanobody, an scFv, a monoclonal antibody, a humanized antibody, a human antibody, a recombinant antibody, a chimeric antibody, or a Fab fragment.

3. The antibody or antigen binding fragment of claim 1, wherein the heavy chain variable region comprises a complementarity determining region 1 (CDR_H1) comprising an amino acid sequence that differs by no more than four amino acids from an amino acid sequence selected from the group consisting of: SEQ ID NOs.: 16, 22, 28, and 34.

4. The antibody or antigen binding fragment of claim 1, wherein the heavy chain variable region comprises a complementarity determining region 1 (CDR_H2) comprising an amino acid sequence that differs by no more than four amino acids from SEQ ID NO: 17, 23, 29, and 35.

5. The antibody or antigen binding fragment of claim 1, wherein the heavy chain variable region comprises a complementarity determining region 1 (CDR_H3) comprising an amino acid sequence that differs by no more than four amino acids from an amino acid sequence selected from the group consisting of: SEQ ID NOs.: 18, 24, 30, and 36.

6. The antibody or antigen binding fragment of claim 1, wherein the light chain variable region comprises a complementarity determining region 1 (CDR_L1) comprising an amino acid sequence that differs by no more than four amino acids from an amino acid sequence selected from the group consisting of: SEQ ID NOs.: SEQ ID NOs: 19, 25, 31, and 37.

7. The antibody or antigen binding fragment of claim 1, wherein the light chain variable region comprises a complementarity determining region 1 (CDR_L2) comprising an amino acid sequence that differs by no more than two amino acids from an amino acid sequence selected from the group consisting of: SEQ ID NOs.: 20, 26, 32, and 38.

8. The antibody or antigen binding fragment of claim 1, wherein the light chain variable region comprises a complementarity determining region 1 (CDR_L3) comprising an amino acid sequence that differs by no more than four amino acids from an amino acid sequence selected from the group consisting of: SEQ ID NOs.: 21, 27, 33, and 39.

9. The antibody or antigen binding fragment of claim 1, wherein the heavy chain variable region comprises a CDR_H1 comprising an amino acid sequence that differs by no more than four amino acids from SEQ ID NO: 16, a CDR_H2 comprising an amino acid sequence that differs by no more than four amino acids from SEQ ID NO: 17, and a CDR_H3 comprising an amino acid sequence that differs by no more than four amino acids from SEQ ID NO: 18.

10. The antibody or antigen binding fragment of claim 1, wherein the heavy chain variable region comprises a CDR_H1 comprising an amino acid sequence that differs by no more than four amino acids from SEQ ID NO: 22, a CDR_H2 comprising an amino acid sequence that differs by no more than four amino acids from SEQ ID NO: 23, and a CDR_H3 comprising an amino acid sequence that differs by no more than four amino acids from SEQ ID NO: 24.

11. The antibody or antigen binding fragment of claim 1, wherein the heavy chain variable region comprises a CDR_H1 comprising an amino acid sequence that differs by no more than four amino acids from SEQ ID NO: 28, a CDR_H2 comprising an amino acid sequence that differs by no more than four amino acids from SEQ ID NO: 29, and a CDR_H3 comprising an amino acid sequence that differs by no more than four amino acids from SEQ ID NO: 30.

12. The antibody or antigen binding fragment of claim 1, wherein the heavy chain variable region comprises a CDR_H1 comprising an amino acid sequence that differs by no more than four amino acids from SEQ ID NO: 34, a CDR_H2 comprising an amino acid sequence that differs by no more than four amino acids from SEQ ID NO: 35, and a CDR_H3 comprising an amino acid sequence that differs by no more than four amino acids from SEQ ID NO: 36.

13. The antibody or antigen binding fragment of claim 1, wherein the light chain variable region comprises a CDR_L1 comprising an amino acid sequence that differs by no more than four amino acids from SEQ ID NO: 19, a CDR_L2 comprising an amino acid sequence that differs by no more than four amino acids from SEQ ID NO: 20, and a CDR_L3 comprising an amino acid sequence that differs by no more than four amino acids from SEQ ID NO: 21.

14. The antibody or antigen binding fragment of claim 1, wherein the light chain variable region comprises a CDR_L1 comprising an amino acid sequence that differs by no more than four amino acids from SEQ ID NO: 25, a CDR_L2 comprising an amino acid sequence that differs by no more than four amino acids from SEQ ID NO: 26, and a CDR_L3 comprising an amino acid sequence that differs by no more than four amino acids from SEQ ID NO: 27.

15. The antibody or antigen binding fragment of claim 1, wherein the light chain variable region comprises a CDR_L1 comprising an amino acid sequence that differs by no more than four amino acids from SEQ ID NO: 31, a CDR_L2 comprising an amino acid sequence that differs by no more than four amino acids from SEQ ID NO: 32, and a CDR_L3 comprising an amino acid sequence that differs by no more than four amino acids from SEQ ID NO: 33.

16. The antibody or antigen binding fragment of claim 1, wherein the light chain variable region comprises a CDR_L1 comprising an amino acid sequence that differs by no more than four amino acids from SEQ ID NO: 37, a CDR_L2 comprising an amino acid sequence that differs by no more than four amino acids from SEQ ID NO: 38, and a CDR_L3 comprising an amino acid sequence that differs by no more than four amino acids from SEQ ID NO: 39.

17. The antibody or antigen binding fragment of any one of claims 1-16, wherein the heavy chain variable region comprises an amino acid sequence having at least 90% identity to an amino acid sequence selected from the group consisting of SEQ ID NOs: 2, 4, or 6.

18. The antibody or antigen binding fragment of any one of claims 1-16, wherein the heavy chain variable region comprises an amino acid sequence encoded by a nucleotide sequence having at least 90% identity to a nucleotide sequence selected from the group consisting of SEQ ID NOs: 3, 5, or 7.

19. The antibody or antigen binding fragment of any one of claims 1-16, wherein the light chain variable region comprises an amino acid sequence having at least 90% identity to an amino acid sequence selected from the group consisting of SEQ ID NOs: 8, 10, 12 or 14.

20. The antibody or antigen binding fragment of any one of claims 1-16, wherein the light chain variable region comprises an amino acid sequence encoded by a nucleotide sequence having at least 90% identity to a nucleotide sequence selected from the group consisting of SEQ ID NOs: 9, 11, 13, or 15.

21. The antibody or antigen binding fragment of any one of claims 1-20, wherein the antibody or antigen binding fragment is a Notch4 inhibitor or a Notch4 neutralizing antibody.

22. The antibody or antigen binding fragment of any one of claims 1-21, wherein the antibody or antigen binding fragment reduces Notch4 activity by at least 20% as compared to Notch4 activity in the absence of the antibody or antigen-binding fragment.

23. The antibody or antigen binding fragment of any one of claims 1-22, wherein the antibody or antigen binding fragment disrupts binding of Notch4 to its cognate ligand.

24. An antibody or antigen binding fragment that binds to Notch4 and comprises:
a) a heavy chain variable region (V_{H}) comprising:
i. a complementarity determining region 1 (CDR_H1) sequence comprising an amino acid sequence that differs by no more than four amino acids from an amino acid sequence selected from the group consisting of SEQ ID NOs 16, 22, 28, and 34; or
ii. a complementarity determining region 2 (CDR_H2) sequence comprising an amino acid sequence that differs by no more than four amino acids from an amino acid sequence selected from the group consisting of SEQ ID NOs 17, 23, 29, and 35; or
iii. a complementarity determining region 3 (CDR_H3) sequence comprising an amino acid sequence that differs by no more than four amino acids from an amino acid sequence selected from the group consisting of SEQ ID NOs 18, 24, 30, and 36; or
b) a light chain variable region (V_{L}) comprising:
i. a complementarity determining region 1 (CDR_L1) sequence comprising an amino acid sequence that differs by no more than four amino acids from an amino acid sequence selected from the group consisting of SEQ ID NOs 19, 25, 31, and 37; or
ii. a complementarity determining region 2 (CDR_L2) sequence comprising an amino acid sequence that differs by no more than two amino acids from an amino acid sequence selected from the group consisting of SEQ ID NOs 20, 26, 32, and 38; or
iii. a complementarity determining region 3 (CDR_L3) sequence comprising an amino acid sequence that differs by no more than four amino acids from an amino acid sequence selected from the group consisting of SEQ ID NOs 21, 27, 33, and 39.

25. A composition comprising an antibody or antigen binding fragment of any one of claim 1-24 or 43-53.

26. The composition of claim 25, wherein the composition comprises a pharmaceutically acceptable excipient or carrier.

27. A cell comprising an antibody or antigen binding fragment of any one of claim 1-24 or 43-53.

28. A polynucleotide comprising a nucleotide sequence encoding an antibody or antigen binding fragment of any one of claim 1-24 or 43-53.

29. The polynucleotide of claim 28, wherein the polynucleotide is comprised in a vector.

30. A composition comprising a polynucleotide claim 28 or 29.

31. A cell comprising a polynucleotide of claim 28 or 29.

32. A kit comprising:
a) an antibody or antigen binding fragment of any one of claim 1-24 or 43-53;
b) a polynucleotide of claim 28 or 29;
c) a composition of claim 25, 26, or 30; or
d) a cell of claim 27 or 31.

33. The kit of claim 32, further comprising instructions for use thereof.

34. A method for reducing Notch4 activity, the method comprising: administering an antibody or antigen binding fragment of any one of claim 1-24 or 43-53, a polynucleotide of claim 28 or 29, or a composition of claim 25, 26, or 30 to a cell.

35. The method of claim 34, wherein said administering to the cell is in vitro.

36. The method of claim 34, wherein said administering to the cell is in vivo.

37. The method of claim 36, wherein said administering to the cell is in a subject.

38. A method for reducing at least one symptom of a disease or disorder associated with increased levels of Notch4, the method comprising administering a therapeutically effective amount of an antibody or antigen binding fragment of any one of claim 1-24 or 43-53, a polynucleotide of claim 28 or 29, or a composition of claim 25, 26, or 30 to a subject in need thereof.

39. The method of claim 38, wherein the disease or disorder associated with an increased level of Notch4 is selected from the group consisting of asthma, allergic disease, and a disease or condition associated with pulmonary viral infection.

40. The method of claim 38 or 39, further comprising, prior to administering, a step of diagnosing the subject with the disease or disorder associated with an increased level of Notch4.

41. The method of any one of claims 38-40, wherein the subject is a mammal.

42. The method of any one of claims 38-41, wherein the subject is human.

43. The antibody or antigen binding fragment of claim 17 wherein the heavy chain variable region comprises an amino acid sequence having at least 91%, at least 92%, at least 93%, at least 94%, at least 95%, at least 96%, at least 97%, at least 98%, or at least 99% identity to an amino acid sequence selected from the group consisting of SEQ ID NOs: 2, 4, or 6.

44. The antibody or antigen binding fragment of claim 43, wherein the heavy chain variable region comprises an amino acid sequence selected from the group consisting of SEQ ID NOs: 2, 4, or 6.

45. The antibody or antigen binding fragment of claim 18, wherein the heavy chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 3, 5, or 7.

46. The antibody or antigen-binding fragment of claim 45, wherein the heavy chain variable region comprises an amino acid sequence encoded by a nucleotide sequence selected from the group consisting of SEQ ID NOS: 3, 5, or 7.

47. The antibody or antigen binding fragment of claim 19, wherein the light chain variable region comprises an amino acid sequence having at least 91%, at least 92%, at least 93%, at least 94%, at least 95%, at least 96%, at least 97%, at least 98%, or at least 99% identity identity to an amino acid sequence selected from the group consisting of SEQ ID NOs: 8, 10, 12 or 14.

48. The antibody or antigen binding fragment of claim 47, wherein the light chain variable region comprises an amino acid sequence selected from the group consisting of SEQ ID NOs: 8, 10, 12, or 14.

49. The antibody or antigen binding fragment of claim 20, wherein the light chain variable region comprises an amino acid sequence encoded by a nucleotide sequence having at least 91%, at least 92%, at least 93%, at least 94%, at least 95%, at least 96%, at least 97%, at least 98%, or at least 99% identity to a nucleotide sequence selected from the group consisting of SEQ ID NOs: 9, 11, 13, or 15.

50. The antibody or antigen binding fragment of claim 49, wherein the light chain variable region comprises an amino acid sequence encoded by a nucleotide sequence selected from the group consisting of SEQ ID NOs: 9, 11, 13, or 15.

51. The antibody or antigen-binding fragment of claim 24, comprising
a) a heavy chain variable region (V_{H}) comprising:
i. a complementarity determining region 1 (CDR_H1) sequence comprising an amino acid sequence that differs by no more than four amino acids from an amino acid sequence selected from the group consisting of SEQ ID NOs 16, 22, 28, and 34;
ii. a complementarity determining region 2 (CDR_H2) sequence comprising an amino acid sequence that differs by no more than four amino acids from an amino acid sequence selected from the group consisting of SEQ ID NOs 17, 23, 29, and 35;
iii. a complementarity determining region 3 (CDR_H3) sequence comprising an amino acid sequence that differs by no more than four amino acids from an amino acid sequence selected from the group consisting of SEQ ID NOs 18, 24, 30, and 36; and
b) a light chain variable region (V_{L}) comprising:
i. a complementarity determining region 1 (CDR_L1) sequence comprising an amino acid sequence that differs by no more than four amino acids from an amino acid sequence selected from the group consisting of SEQ ID NOs 19, 25, 31, and 37;
ii. a complementarity determining region 2 (CDR_L2) sequence comprising an amino acid sequence that differs by no more than two amino acids from an amino acid sequence selected from the group consisting of SEQ ID NOs 20, 26, 32, and 38;
iii. a complementarity determining region 3 (CDR_L3) sequence comprising an amino acid sequence that differs by no more than four amino acids from an amino acid sequence selected from the group consisting of SEQ ID NOs 21, 27, 33, and 39.

52. The antibody of claim 51, wherein the antibody or antigen binding fragment thereof comprise an amino acid sequence whose CDR sequences that differ by no more than twelve, no more than eleven, no more than ten, no more than nine, no more than eight, no more than seven, no more than six, no more than five, no more than four, no more than three, no more than two, or no more than one amino acid from an antibody or antigen binding fragment comprising:
a) a heavy chain variable region (V_{H}) comprising:
i. a complementarity determining region 1 (CDR_H1) sequence comprising an amino acid sequence selected from the group consisting of SEQ ID NOs 16, 22, 28, and 34;
ii. a complementarity determining region 2 (CDR_H2) sequence comprising an amino acid sequence selected from the group consisting of SEQ ID NOs 17, 23, 29, and 35;
iii. a complementarity determining region 3 (CDR_H3) sequence comprising an amino acid sequence selected from the group consisting of SEQ ID NOs 18, 24, 30, and 36; and
b) a light chain variable region (V_{L}) comprising:
i. a complementarity determining region 1 (CDR_L1) sequence comprising an amino acid sequence selected from the group consisting of SEQ ID NOs 19, 25, 31, and 37;
ii. a complementarity determining region 2 (CDR_L2) sequence comprising an amino acid sequence selected from the group consisting of SEQ ID NOs 20, 26, 32, and 38;
iii. a complementarity determining region 3 (CDR_L3) sequence comprising an amino acid sequence selected from the group consisting of SEQ ID NOs 21, 27, 33, and 39.
iv.

53. The antibody of claim 52, wherein the antibody or antigen binding fragment thereof comprise:
a) a heavy chain variable region (V_{H}) comprising:
i. a complementarity determining region 1 (CDR_H1) sequence comprising an amino acid sequence selected from the group consisting of SEQ ID NOs 16, 22, 28, and 34;
ii. a complementarity determining region 2 (CDR_H2) sequence comprising an amino acid sequence selected from the group consisting of SEQ ID NOs 17, 23, 29, and 35;
iii. a complementarity determining region 3 (CDR_H3) sequence comprising an amino acid sequence selected from the group consisting of SEQ ID NOs 18, 24, 30, and 36; and
b) a light chain variable region (V_{L}) comprising:
i. a complementarity determining region 1 (CDR_L1) sequence comprising an amino acid sequence selected from the group consisting of SEQ ID NOs 19, 25, 31, and 37;
ii. a complementarity determining region 2 (CDR_L2) sequence comprising an amino acid sequence selected from the group consisting of SEQ ID NOs 20, 26, 32, and 38;
iii. a complementarity determining region 3 (CDR_L3) sequence comprising an amino acid sequence selected from the group consisting of SEQ ID NOs 21, 27, 33, and 39.

54. The method of any one of claims 38-42, wherein the at least one symptom comprises tissue inflammation.

55. The method of any one of claims 38-42 and 54, wherein the at least one symptom comprises a symptom selected from the group consisting of macrophage skewing, neutrophil infiltration, production of an inflammatory cytokine, and combinations thereof.

56. The method of claim 55, wherein the inflammatory cytokine is IL-6 or IFNγ.

57. The antibody or antigen-binding fragment of claim 24, comprising a heavy chain variable region (VH) and a light chain variable region (VL),
wherein the heavy chain variable region comprises complementarity determining regions CDR_H1, CDR_H2, and CDR_H3,
wherein the light chain variable region comprises CDR_L1, CDR_L2, and CDR_L3, and wherein
(a) (i) CDR_H1 comprises an amino acid sequence that differs by no more than two amino acid residues from that of SEQ ID NO: 16,
(ii) CDR_H2 comprises an amino acid sequence that differs by no more than two amino acid residues from that of SEQ ID NO: 17,
(iii) CDR_H3 comprises an amino acid sequence that differs by no more than two amino acid residues from that of SEQ ID NO: 18,
(iv) CDR_L1 comprises an amino acid sequence that differs by no more than two amino acid residues from that of SEQ ID NO: 19,
(v) CDR_L2 comprises an amino acid sequence that differs by no more than two amino acid residues from that of SEQ ID NO: 20; and
(vi) CDR_L3 comprises an amino acid sequence that differs by no more than two amino acid residues from that of SEQ ID NO: 21; or
(b) (i) CDR_H1 comprises an amino acid sequence that differs by no more than two amino acid residues from that of SEQ ID NO: 22,
(ii) CDR_H2 comprises an amino acid sequence that differs by no more than two amino acid residues from that of SEQ ID NO: 23,
(iii) CDR_H3 comprises an amino acid sequence that differs by no more than two amino acid residues from that of SEQ ID NO: 24,
(iv) CDR_L1 comprises an amino acid sequence that differs by no more than two amino acid residues from that of SEQ ID NO: 25,
(v) CDR_L2 comprises an amino acid sequence that differs by no more than two amino acid residues from that of SEQ ID NO: 26; and
(vi) CDR_L3 comprises an amino acid sequence that differs by no more than two amino acid residues from that of SEQ ID NO: 27; or
(c) (i) CDR_H1 comprises an amino acid sequence that differs by no more than two amino acid residues from that of SEQ ID NO: 28,
(ii) CDR_H2 comprises an amino acid sequence that differs by no more than two amino acid residues from that of SEQ ID NO: 29,
(iii) CDR_H3 comprises an amino acid sequence that differs by no more than two amino acid residues from that of SEQ ID NO: 30,
(iv) CDR_L1 comprises an amino acid sequence that differs by no more than two amino acid residues from that of SEQ ID NO: 31,
(v) CDR_L2 comprises an amino acid sequence that differs by no more than two amino acid residues from that of SEQ ID NO: 32; and
(vi) CDR_L3 comprises an amino acid sequence that differs by no more than two amino acid residues from that of SEQ ID NO: 33; or
(d) (i) CDR_H1 comprises an amino acid sequence that differs by no more than two amino acid residues from that of SEQ ID NO: 34,
(ii) CDR_H2 comprises an amino acid sequence that differs by no more than two amino acid residues from that of SEQ ID NO: 35,
(iii) CDR_H3 comprises an amino acid sequence that differs by no more than two amino acid residues from that of SEQ ID NO: 36,
(iv) CDR_L1 comprises an amino acid sequence that differs by no more than two amino acid residues from that of SEQ ID NO: 37,
(v) CDR_L2 comprises an amino acid sequence that differs by no more than two amino acid residues from that of SEQ ID NO: 38; and
(vi) CDR_L3 comprises an amino acid sequence that differs by no more than two amino acid residues from that of SEQ ID NO: 39.

58. The antibody or antigen-binding fragment of claim 57, comprising
(a) (i) CDR_H1 comprises an amino acid sequence of SEQ ID NO: 16,
(ii) CDR_H2 comprises an amino acid sequence of SEQ ID NO: 17,
(iii) CDR_H3 comprises an amino acid sequence of SEQ ID NO: 18,
(iv) CDR_L1 comprises an amino acid sequence of SEQ ID NO: 19,
(v) CDR_L2 comprises an amino acid sequence of SEQ ID NO: 20; and
(vi) CDR_L3 comprises an amino acid sequence of SEQ ID NO: 21; or
(b) (i) CDR_H1 comprises an amino acid sequence of SEQ ID NO: 22,
(ii) CDR_H2 comprises an amino acid sequence of SEQ ID NO: 23,
(iii) CDR_H3 comprises an amino acid sequence of SEQ ID NO: 24,
(iv) CDR_L1 comprises an amino acid sequence of SEQ ID NO: 25,
(v) CDR_L2 comprises an amino acid sequence of SEQ ID NO: 26; and
(vi) CDR_L3 comprises an amino acid sequence of SEQ ID NO: 27; or
(c) (i) CDR_H1 comprises an amino acid sequence of SEQ ID NO: 28,
(ii) CDR_H2 comprises an amino acid sequence of SEQ ID NO: 29,
(iii) CDR_H3 comprises an amino acid sequence of SEQ ID NO: 30,
(iv) CDR_L1 comprises an amino acid sequence of SEQ ID NO: 31,
(v) CDR_L2 comprises an amino acid sequence of SEQ ID NO: 32; and
(vi) CDR_L3 comprises an amino acid sequence of SEQ ID NO: 33; or
(d) (i) CDR_H1 comprises an amino acid sequence of SEQ ID NO: 34,
(ii) CDR_H2 comprises an amino acid sequence of SEQ ID NO: 35,
(iii) CDR_H3 comprises an amino acid sequence of SEQ ID NO: 36,
(iv) CDR_L1 comprises an amino acid sequence of SEQ ID NO: 37,
(v) CDR_L2 comprises an amino acid sequence of SEQ ID NO: 38; and
(vi) CDR_L3 comprises an amino acid sequence of SEQ ID NO: 39.


SEQUENCE LISTING (57 sequences)

SEQ ID NO: 1 - DNA, 5912 bp, Homo sapiens
atgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttg

SEQ ID NO: 2 - protein, 116 aa, Artificial Sequence
  Description of Artificial Sequence Synthetic polypeptide
QVQLQQPGAELVKPGASVKLSCKASGYTFINYWMLWVKQRPGQGLEWIGMIHPNSVSTNYNEKFKSKATLTVDKSSSTAYMQLSSLTSEDSAVYYCGTGRDYFDYWGQGTTLTVSS

SEQ ID NO: 3 - DNA, 348 bp, Artificial Sequence
  Description of Artificial Sequence Synthetic polynucleotide
caggtccaacttcaacagcctggggctgagctggtaaagcctggggcttcagtgaagttgtcctgcaaggcttctggctacacttttatcaattactggatgctctgggtgaagcagaggcctggacaaggccttgagtggattggtatgattcatcctaatagtgttagtactaactacaatgagaagttcaagagcaaggccacactgactgtagacaaatcctccagcacagcctacatgcaactcagcagcctgacatctgaggactctgcggtctattactgtggaactgggagggactactttgactactggggccaaggcaccactctcacagtctcctca

SEQ ID NO: 4 - protein, 119 aa, Artificial Sequence
  Description of Artificial Sequence Synthetic polypeptide
QVTLKESGPGILQPSQTLNLTCSFSGFSLSTFGMGVGWIRQPSGKGLEWLAHIWWDDEMYYNPVLKSRLTISKETSKTQVFLKIATVDTADTATYYCVRRRRGLAMDSWGHGTSVTVSS

SEQ ID NO: 5 - DNA, 357 bp, Artificial Sequence
  Description of Artificial Sequence Synthetic polynucleotide
caggttactctgaaagagtctggccctgggatattgcagccctcccagaccctcaatctgacttgttctttctctgggttttcactgagcacttttggtatgggtgtgggctggattcgtcagccttcagggaagggtctggagtggctggcacacatttggtgggatgatgaaatgtactataacccagtcctgaagagtcggctcacaatctccaaggaaacctccaaaacccaggtattcctcaagatcgccactgtggacactgcagatactgccacatattactgtgttcgaagacgacgcgggttggctatggactcctggggtcacggaacctcagtcaccgtctcctca

SEQ ID NO: 6 - protein, 112 aa, Artificial Sequence
  Description of Artificial Sequence Synthetic polypeptide
DVQLQESGPGLVKPSQSLSLTCSVTGYSITRGFYWNWIRQFPGNKLEWMGYISYDGSNNYNPSLKNRISITRDTSKNQFFLQLNSVTTEDTATYYCARMDYWGQGTSVTVSS

SEQ ID NO: 7 - DNA, 336 bp, Artificial Sequence
  Description of Artificial Sequence Synthetic polynucleotide
gatgtacagcttcaggagtcaggacctggcctcgtgaaaccttctcagtctctgtctctcacctgctctgtcactggctactccatcaccagaggtttttactggaactggatccggcagtttccaggaaacaaactggaatggatgggctacataagctacgacggtagcaataactacaacccatctctcaaaaatcgaatctccatcactcgtgacacatctaagaaccagtttttcctgcagttgaattctgtgactactgaggacacagctacatattactgtgcaaggatggactactggggtcaaggaacctcagtcaccgtctcctca

SEQ ID NO: 8 - protein, 107 aa, Artificial Sequence
  Description of Artificial Sequence Synthetic polypeptide
DIVMTQSHKFMSTSVGDRVSITCKASQDVGTAVAWYQQKPGQSPKLLIYWASTRHTGVPDRFTGSGSGTDFTLTIGNVQSEDLADYFCQQYSSYPFTFGSGTKLEIK

SEQ ID NO: 9 - DNA, 321 bp, Artificial Sequence
  Description of Artificial Sequence Synthetic polynucleotide
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattggcaatgtgcagtctgaagacttggcagattatttctgtcagcaatatagcagctatccattcacgttcggctcggggacaaagttggaaataaaa

SEQ ID NO: 10 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 11 - DNA, 321 bp, Artificial Sequence
  Description of Artificial Sequence Synthetic polynucleotide
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaa

SEQ ID NO: 12 - protein, 107 aa, Artificial Sequence
  Description of Artificial Sequence Synthetic polypeptide
DIVMTQSHKFMSTSIGDRVNITCKASQDVTTAVAWYQQKPGQSPKLLIYSASYRYTGIPDRFTGTGSGTDFTFTISTVQAEDLALYYCQQHSNTPLTFGAGTKLELK

SEQ ID NO: 13 - DNA, 321 bp, Artificial Sequence
  Description of Artificial Sequence Synthetic polynucleotide
gacattgtgatgacccagtctcacaaattcatgtccacatcaataggagacagggtcaacatcacctgcaaggccagtcaggatgtgactactgctgtagcctggtatcaacagaaaccaggacaatctcctaaactactgatttactcggcatcctaccgatacactggaatccctgatcgcttcactggcactggatctgggacggatttcactttcaccatcagcactgtgcaggctgaagacctggcactttattactgtcagcaacattctaatactccgctcacgttcggtgctgggaccaagctggagctgaaa

SEQ ID NO: 14 - protein, 108 aa, Artificial Sequence
  Description of Artificial Sequence Synthetic polypeptide
EIVLTQSPALMAASPGEKVTITCSVSSSISSSNLHWYQQKSETSPKPWIYGTSNLASGVPVRFSGSGSGTSYSLTISSMEAEDAATYYCQQWSSYPFTFGSGTKLEIK

SEQ ID NO: 15 - DNA, 324 bp, Artificial Sequence
  Description of Artificial Sequence Synthetic polynucleotide
gaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccattcacgttcggctcggggacaaagttggaaataaaa

SEQ ID NO: 16 - protein, 8 aa, Artificial Sequence
  Description of Artificial Sequence Synthetic peptide
GYTFINYW

SEQ ID NO: 17 - protein, 8 aa, Artificial Sequence
  Description of Artificial Sequence Synthetic peptide
IHPNSVST

SEQ ID NO: 18 - protein, 9 aa, Artificial Sequence
  Description of Artificial Sequence Synthetic peptide
GTGRDYFDY

SEQ ID NO: 19 - protein, 6 aa, Artificial Sequence
  Description of Artificial Sequence Synthetic peptide
QDVGTA

SEQ ID NO: 20 - protein, 3 aa, Artificial Sequence
  Description of Artificial Sequence Synthetic peptide
WAS

SEQ ID NO: 21 - protein, 9 aa, Artificial Sequence
  Description of Artificial Sequence Synthetic peptide
QQYSSYPFT

SEQ ID NO: 22 - protein, 8 aa, Artificial Sequence
  Description of Artificial Sequence Synthetic peptide
AYTFSDHS

SEQ ID NO: 23 - protein, 8 aa, Artificial Sequence
  Description of Artificial Sequence Synthetic peptide
INTETGEP

SEQ ID NO: 24 - protein, 10 aa, Artificial Sequence
  Description of Artificial Sequence Synthetic peptide
TRGGHYFFDY

SEQ ID NO: 25 - protein, 6 aa, Artificial Sequence
  Description of Artificial Sequence Synthetic peptide
QDISNY

SEQ ID NO: 26 - protein, 3 aa, Artificial Sequence
  Description of Artificial Sequence Synthetic peptide
YTS

SEQ ID NO: 27 - protein, 9 aa, Artificial Sequence
  Description of Artificial Sequence Synthetic peptide
QQGNTLPWT

SEQ ID NO: 28 - protein, 10 aa, Artificial Sequence
  Description of Artificial Sequence Synthetic peptide
GFSLSTFGMG

SEQ ID NO: 29 - protein, 7 aa, Artificial Sequence
  Description of Artificial Sequence Synthetic peptide
IWWDDEM

SEQ ID NO: 30 - protein, 11 aa, Artificial Sequence
  Description of Artificial Sequence Synthetic peptide
VRRRRGLAMDS

SEQ ID NO: 31 - protein, 6 aa, Artificial Sequence
  Description of Artificial Sequence Synthetic peptide
QDVTTA

SEQ ID NO: 32 - protein, 3 aa, Artificial Sequence
  Description of Artificial Sequence Synthetic peptide
SAS

SEQ ID NO: 33 - protein, 9 aa, Artificial Sequence
  Description of Artificial Sequence Synthetic peptide
QQHSNTPLT

SEQ ID NO: 34 - protein, 9 aa, Artificial Sequence
  Description of Artificial Sequence Synthetic peptide
GYSITRGFY

SEQ ID NO: 35 - protein, 7 aa, Artificial Sequence
  Description of Artificial Sequence Synthetic peptide
ISYDGSN

SEQ ID NO: 36 - protein, 5 aa, Artificial Sequence
  Description of Artificial Sequence Synthetic peptide
ARMDY

SEQ ID NO: 37 - protein, 7 aa, Artificial Sequence
  Description of Artificial Sequence Synthetic peptide
SSISSSN

SEQ ID NO: 38 - protein, 3 aa, Artificial Sequence
  Description of Artificial Sequence Synthetic peptide
GTS

SEQ ID NO: 39 - protein, 9 aa, Artificial Sequence
  Description of Artificial Sequence Synthetic peptide
QQWSSYPFT

SEQ ID NO: 40 - protein, 224 aa, Artificial Sequence
  Description of Artificial Sequence Synthetic polypeptide
MRSQFSLQLLSTQDLTVGWSYIILFLVATATGVHSQVQLQQPGAELVKPGASVKLSCKASGYTFINYWMLWVKQRPGQGLEWIGMIHPNSVSTNYNEKFKSKATLTVDKSSSTAYMQLSSLTSEDSAVYYCGTGRDYFDYWGQGTTLTVSSAKTTPPSVYPLAPGSAAQTNSMVTLGCLVKGYFPEPVTVTWNSGSLSSGVHTFPAVLQSDLYTLSSSVTVPSS

SEQ ID NO: 41 - DNA, 672 bp, Artificial Sequence
  Description of Artificial Sequence Synthetic polynucleotide
atgagatcacagttctctctacagttactgagcacacaggacctcacagtgggatggagctatatcatcctctttttggtagcaacagctacaggtgtccactcccaggtccaacttcaacagcctggggctgagctggtaaagcctggggcttcagtgaagttgtcctgcaaggcttctggctacacttttatcaattactggatgctctgggtgaagcagaggcctggacaaggccttgagtggattggtatgattcatcctaatagtgttagtactaactacaatgagaagttcaagagcaaggccacactgactgtagacaaatcctccagcacagcctacatgcaactcagcagcctgacatctgaggactctgcggtctattactgtggaactgggagggactactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagc

SEQ ID NO: 42 - protein, 211 aa, Artificial Sequence
  Description of Artificial Sequence Synthetic polypeptide
MGRLTSSFLLLTVPAYVLSQVTLKESGPGILQPSQTLNLTCSFSGFSLSTFGMGVGWIRQPSGKGLEWLAHIWWDDEMYYNPVLKSRLTISKETSKTQVFLKIATVDTADTATYYCVRRRRGLAMDSWGHGTSVTVSSAKTTPPSVYPLAPGSAAQTNSMVTLGCLVKGYFPEPVTVTWNSGSLSSGVHTFPAVLQSDLYTLSSSVTVPSS

SEQ ID NO: 43 - DNA, 635 bp, Artificial Sequence
  Description of Artificial Sequence Synthetic polynucleotide
atgggcaggcttacttcttcattcctgttactgactgtccctgcatatgtcctgtcccaggttactctgaaagagtctggccctgggatattgcagccctcccagaccctcaatctgacttgttctttctctgggttttcactgagcacttttggtatgggtgtgggctggattcgtcagccttcagggaagggtctggagtggctggcacacatttggtgggatgatgaaatgtactataacccagtcctgaagagtcggctcacaatctccaaggaaacctccaaaacccaggtattcctcaagatcgccactgtggacactgcagatactgccacatattactgtgttcgaagacgacgcgggttggctatggactcctggggtcacggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagctg

SEQ ID NO: 44 - protein, 203 aa, Artificial Sequence
  Description of Artificial Sequence Synthetic polypeptide
MKVLSLLYLLTAIPGILSDVQLQESGPGLVKPSQSLSLTCSVTGYSITRGFYWNWIRQFPGNKLEWMGYISYDGSNNYNPSLKNRISITRDTSKNQFFLQLNSVTTEDTATYYCARMDYWGQGTSVTVSSAKTTPPSVYPLAPGSAAQTNSMVTLGCLVKGYFPEPVTVTWNSGSLSSGVHTFPAVLQSDLYTLSSSVTVPSS

SEQ ID NO: 45 - DNA, 611 bp, Artificial Sequence
  Description of Artificial Sequence Synthetic polynucleotide
atgaaagtgttgagtctgttgtacctgttgacagccattcctggtatcctgtctgatgtacagcttcaggagtcaggacctggcctcgtgaaaccttctcagtctctgtctctcacctgctctgtcactggctactccatcaccagaggtttttactggaactggatccggcagtttccaggaaacaaactggaatggatgggctacataagctacgacggtagcaataactacaacccatctctcaaaaatcgaatctccatcactcgtgacacatctaagaaccagtttttcctgcagttgaattctgtgactactgaggacacagctacatattactgtgcaaggatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagctg

SEQ ID NO: 46 - protein, 203 aa, Artificial Sequence
  Description of Artificial Sequence Synthetic polypeptide
METHSQVFVYTLLWLSGVEGDIVMTQSHKFMSTSVGDRVSITCKASQDVGTAVAWYQQKPGQSPKLLIYWASTRHTGVPDRFTGSGSGTDFTLTIGNVQSEDLADYFCQQYSSYPFTFGSGTKLEIKRADAAPTVSIFPPSSEQLTSGGASVVCFLNNFYPKDINVKWKIDGSERQNGVLNSWTDQDSKDSTYSMSSTLTLTK

SEQ ID NO: 47 - DNA, 609 bp, Artificial Sequence
  Description of Artificial Sequence Synthetic polynucleotide
atggagacacattctcaggtctttgtatacacgttgctgtggttgtctggtgttgaaggagacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattggcaatgtgcagtctgaagacttggcagattatttctgtcagcaatatagcagctatccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 48 - protein, 202 aa, Artificial Sequence
  Description of Artificial Sequence Synthetic polypeptide
MSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPKDINVKWKIDGSERQNGVLNSWTDQDSKDSTYSMSSTLTLTK

SEQ ID NO: 49 - DNA, 606 bp, Artificial Sequence
  Description of Artificial Sequence Synthetic polynucleotide
atgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 50 - protein, 203 aa, Artificial Sequence
  Description of Artificial Sequence Synthetic polypeptide
MESQIQVFVFVFLWLSGVDGDIVMTQSHKFMSTSIGDRVNITCKASQDVTTAVAWYQQKPGQSPKLLIYSASYRYTGIPDRFTGTGSGTDFTFTISTVQAEDLALYYCQQHSNTPLTFGAGTKLELKRADAAPTVSIFPPSSEQLTSGGASVVCFLNNFYPKDINVKWKIDGSERQNGVLNSWTDQDSKDSTYSMSSTLTLTK

SEQ ID NO: 51 - DNA, 609 bp, Artificial Sequence
  Description of Artificial Sequence Synthetic polynucleotide
atggagtcacagattcaggtctttgtattcgtgtttctctggttgtctggtgttgacggagacattgtgatgacccagtctcacaaattcatgtccacatcaataggagacagggtcaacatcacctgcaaggccagtcaggatgtgactactgctgtagcctggtatcaacagaaaccaggacaatctcctaaactactgatttactcggcatcctaccgatacactggaatccctgatcgcttcactggcactggatctgggacggatttcactttcaccatcagcactgtgcaggctgaagacctggcactttattactgtcagcaacattctaatactccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 52 - protein, 206 aa, Artificial Sequence
  Description of Artificial Sequence Synthetic polypeptide
MDFHVQIFSFMLISVTVILSSGEIVLTQSPALMAASPGEKVTITCSVSSSISSSNLHWYQQKSETSPKPWIYGTSNLASGVPVRFSGSGSGTSYSLTISSMEAEDAATYYCQQWSSYPFTFGSGTKLEIKRADAAPTVSIFPPSSEQLTSGGASVVCFLNNFYPKDINVKWKIDGSERQNGVLNSWTDQDSKDSTYSMSSTLTLTK

SEQ ID NO: 53 - DNA, 618 bp, Artificial Sequence
  Description of Artificial Sequence Synthetic polynucleotide
atggattttcatgtgcagattttcagcttcatgctaatcagtgtcacagtcatattgtccagtggagaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 54 - DNA, 20 bp, Artificial Sequence
  Description of Artificial Sequence Synthetic primer
gggggaagctctccactaga

SEQ ID NO: 55 - DNA, 20 bp, Artificial Sequence
  Description of Artificial Sequence Synthetic primer
gttgtgtgctgatttggccc

SEQ ID NO: 56 - DNA, 19 bp, Artificial Sequence
  Description of Artificial Sequence Synthetic primer
cggtccaaattcctgctga

SEQ ID NO: 57 - DNA, 20 bp, Artificial Sequence
  Description of Artificial Sequence Synthetic primer
cattgggttccttccatcca